(19) United States
(12) Patent Application Publication
(10) Pub. No.: US 2025/0109126 A1
(43) Pub. Date: Apr. 3, 2025

(54) DIHYDRO-OXAZOL DERIVATIVE COMPOUNDS

(71) Applicant: AC IMMUNE SA, Lausanne (CH)

(72) Inventors: Véronique Dehlinger, Lausanne (CH); Emanuele Gabellieri, Lausanne (CH)

(21) Appl. No.: 18/722,753

(22) PCT Filed: Dec. 22, 2022
(86) PCT No.: PCT/EP2022/087637

§ 371 Date: Jun. 21, 2024

(30) Foreign Application Priority Data
Dec. 22, 2021 (EP) 21217126.8

(51) Int. Cl. C07D 413/14; A61K 31/422; A61K 31/4439; C07D 413/04

(52) U.S. Cl. CPC C07D 413/14; A61K 31/422; A61K 31/4439; C07D 413/04

(57) ABSTRACT

The present invention relates to novel compounds for the treatment, alleviation or prevention of a group of diseases, disorders and abnormalities which are responsive to the modulation or inhibition of the activation of a component of the NLRP3 inflammasome pathway. In particular, the component of the inflammasome pathway is a NOD-like receptor (NLR) family, pyrin domain-containing protein 3 (NLRP3) inflammasome. More particularly, the compounds of the present invention have the capability to modulate the NLRP3 inflammasome pathway. Further, the compounds of the present invention are suitable for the treatment, alleviation or prevention of a group of diseases, disorders and abnormalities which are responsive to the modulation, in particular decrease. IL-1 beta and/or IL-18 levels.

### FIELD OF THE INVENTION

The present invention relates to novel compounds that are useful for the treatment, alleviation or prevention of a disease, disorder or abnormality which is responsive to the modulation, in particular inhibition of the activation, of a component of the NLRP3 inflammasome pathway. In particular, the component of the inflammasome pathway is NOD-like receptor (NLR) family, pyrin domain-containing protein 3 (NLRP3) inflammasome. More particularly, the compounds of the present invention have the capability to modulate, e.g., inhibit the activation of, the NLRP3 inflammasome pathway. Further, the compounds of the present invention have the capability to modulate, in particular decrease, IL-1 beta and/or IL-18 levels. The present invention relates to novel compounds for the treatment, alleviation or prevention of a disease, disorder or abnormality which is responsive to the inhibition of the activation of the NLRP3 inflammasome pathway. The present invention relates to novel compounds for the treatment, alleviation or prevention of a disease, disorder or abnormality which is responsive to the modulation of IL-1 beta and/or IL-18 levels. The present invention relates to pharmaceutical compositions comprising said compounds, methods of using said compounds in the treatment of various diseases, disorders or abnormalities which is responsive to the above-mentioned modulation, medicaments containing them and their uses thereof.

### BACKGROUND OF THE INVENTION

Inflammasome protein complexes are the key components of inflammatory signalling. These complexes assemble in response to various danger signals such as molecules from infectious agents (pathogen-associated molecular patterns, PAMPs) as well as altered host molecules, products of sterile tissue damage and environmental factors (danger associated molecular patterns, DAMPs). The inflammasome family consists of NALP1-14, IPAF, and NAIP 1-6, with each family member providing specificity towards different PAMPs/DAMPs including nucleic acids, bacterial proteins, metabolites, protein aggregates and the activity of toxins (Sharma, D. & Kanneganti, T. D. The cell biology of inflammasomes: mechanisms of inflammasome activation and regulation. J. Cell Biol. 213, 617-629 (2016)). Inflammasomes are typically composed of a sensor (a cytosolic pattern-recognition receptor, PRR) and an adaptor protein called apoptosis associated speck-like protein containing a caspase-recruitment domain (CARD) (ASC), and an effector such as the protease caspase-1 (Broz, P.; Dixit, V. M. Inflammasomes: Mechanism of Assembly, Regulation and Signalling. Nat. Rev. Immunol. 2016, 16, 407-420).

NLRP3 (NOD-like receptor (NLR) family, pyrin domain-containing protein 3) inflammasome is one of the best-described family members. It is a tripartite protein of the NLR family and contains an amino-terminal PYRIN (PYD) domain, a nucleotide-binding NACHT domain and a carboxy-terminal leucine-rich repeat (LRR) domain. In response to various agents including aggregated proteins, crystals and altered cellular ion homeostasis, the NLRP3 sensor molecule assembles into a multi-molecular complex with apoptosis-associated speck-like protein containing a caspase activation and recruitment domain (ASC aka PYCARD) adaptor protein. ASC protein polymerization into a large complex (ASC speck) leads to activation of caspase-1 effector protein and subsequent cleavage of pro-IL-1 beta (β) and pro-IL18 into their active secreted forms and mediates pyroptosis (Heneka et al., 2018 Nat Rev Neurosci). IL-1 beta (β) acts through IL-1 beta (β) receptors, induces secondary pro-inflammatory signals including IL-6 and TNF alpha secretion, and attracts and activates cells of adaptive immune system at the sites of infection. NLRP3/ASC complexes seems to be released into the extracellular environment where they can propagate inflammation.

Multiple genetic and pharmacological evidence highlight the importance of NLRP3 inflammasome in human disease. NLRP3 gain-of-function mutations lead to the inherited cryopyrin-associated periodic syndromes (CAPS) including Muckle-Wells syndrome (MWS), familial cold auto-inflammatory syndrome (FCAS) and neonatal-onset multisystem inflammatory disease (NOMID).

Accumulation of tissue damage products associated with ageing results in activation of NLRP3 inflammasome in multiple diseases including metabolic disorders, Alzheimer's disease, Parkinson's disease, amyotrophic lateral sclerosis, atherosclerosis, obesity, lung diseases, liver diseases and gout.

Vast experimental evidence from animal models points out the detrimental role of excessive NLRP3 activation in a wide spectrum of diseases. NLRP3-inflammasome genetic or pharmacological downregulation showed protection in models of Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 and type 2 diabetes, rheumatoid arthritis, myelodysplastic syndrome, among others (Heneka et al., Nat. Rev. Neurosci. 2018 October; 19(10):610-621; Mangan et al., Nat. Rev. Drug Discov. 2018 August; 17(8):588-606).

For the reasons described above modulation of NLRP3 inflammasome pathway activity represents a promising therapeutic approach.

Current treatments for NLRP3-related diseases include biologics targeting IL-1. These are the recombinant IL-1 receptor antagonist anakinra, the neutralizing IL-1 beta (β) antibody canakinumab and the soluble decoy IL-1 receptor rilonacept. However, their activity is limited to downstream effectors of inflammasome and their bioavailability for central nervous system (CNS) applications is limited.

Several small molecules have been shown to inhibit the NLRP3 inflammasome pathway (Baldwin, A. G., Brough, D. & Freeman, S. Inhibiting the NLRP3 inflammasome pathway: a chemical perspective. J. Med. Chem. 59, 1691-1710 (2016); reviewed in Mangan et al., Nat Rev Drug Discov. 2018 August; 17(8):588-606). These include various chemical classes such as sulfonylurea-based compounds (glyburide, CP-456,773 (aka CRID3 and MCC950) and its derivatives); fenamate classes of non-steroidal anti-inflammatory drugs; hydroxysulfonamide analogue JC-171; novel boron compound series; benzimidazole-containing structure Fc11a-2; polyketide spirodalesol; acrylate and acrylamide derivatives; 3,4-methylenedioxy-β-nitrostyrene; β-sulfonyl nitrile molecule OLT1177; CY-09; BOT-4-one; and Michael acceptors. Most of these compounds have a promiscuous mode of action and limited potency.

WO2016131098, WO2017/140778 and WO2018215818 refer to sulfonylurea and related compounds and their use in treating or identifying a disease or condition responsive to inhibition of NLRP3 or inhibition of the activation of NLRP3 or related components of the inflammatory process.

WO2019008025, WO2019008029, WO2019034686, WO2019034688, WO2019034690, WO2019034692, WO2019034693, WO2019034696, WO2019034697, WO2019068772, WO2019092170, WO2019092171 and WO2019092172 refer to novel compounds (e.g. sulfonylureas, sulfonylthioureas, sulfoximine ureas and sulfoximine thioureas), useful in the treatment and prevention of medical disorders and diseases, most especially by NLRP3 inhibition.

WO2017184604, WO2017184623, WO2017184624, WO2019023145, WO2019023147 and WO2019079119 refer to chemical entities that are useful for treating a condition, disease, or disorder in which a decrease or increase in NLRP3 activity contributes to the pathology and/or symptoms and/or progression of the condition, disease, or disorder in a subject.

WO2019211463, WO2020021447, and WO2021043966, WO2021239885, WO2021219784, WO2021214284, WO2021209552, WO2021209539 disclose compounds for inhibiting NLRP3 and/or NLRP3 inflammasome pathway.

WO2018136890 refers to sulfonylurea and sulfonyl thiourea compounds and their use in treating a disease or condition responsive to modulation of cytokines such as IL-1 beta (β) and IL-18, modulation of NLRP3 or inhibition of the activation of NLRP3 or related components of the inflammatory process.

WO2018225018 and WO2019043610 refer to NLRP3 modulators as well as to the use of the novel inhibitor compounds in the treatment of diseases or conditions as well as treatment of disease states mediated by NLRP3 as well as treatment of diseases or conditions in which interleukin 1 beta (β) activity and interleukin-18 (IL-18) are implicated.

WO2018015445 refers to sulfonylurea compounds which possess inflammasome inhibitory activity and are accordingly useful in methods of treatment of the human or animal body.

WO2020018975 discloses sulfonimidamide derivatives defined as inhibitors of interleukin-1 activity and NLRP3 modulators in connection with inflammatory processes.

WO9832733 refers to aryl and heteroaryl substituted sulfonyl ureas that are inhibitors of interleukin-1 alpha (α) and interleukin-1 beta (β) processing and release.

WO2020018970 discloses sulfonylureas defined as inhibitors of interleukin-1 activity.

The crosstalk between the NLRP3 inflammasome pathway and Tau pathology has been recently deciphered. Ising et al. (Nature 2019 November; 575 (7784): 669-673) investigated the important role of microglia and NLRP3 inflammasome pathway activation in the pathogenesis of tauopathies in the Tau22 mouse model of FrontoTemporal Dementia (FTD). Genetic ablation of components of the NLRP3 inflammasome pathway in Tau22 mice reduced Tau aggregation/phosphorylation as well as improved cognition. Stancu et al. (Acta Neuropathol. 2019; 137(4):599-617) investigated the role of inflammasome activation in prion-like or templated seeding of Tau pathology. Significant inhibition of exogenously seeded Tau pathology was found in ASC deficient-PS19 Tau transgenic mice. Furthermore, it was demonstrated that chronic intracerebral administration of the NLRP3 inhibitor, MCC950, inhibits exogenously seeded Tau pathology. Finally, ASC deficiency also decreased non-exogenously seeded Tau pathology in PS19 mice.

There is a need to identify and develop specific NLRP3 inflammasome pathway inhibitors and/or modulators of interleukin activity with improved pharmacological and/or physiological and/or physicochemical properties.

The present invention provides compound of formula (I) which have surprisingly been found to be capable of modulating a component of the NLRP3 inflammasome pathway, in particular inhibiting the activation, of a component of the NLRP3 inflammasome pathway, such as NLRP3 inflammasome. Thus, such compounds are beneficial in the treatment of a disease, disorder, or abnormality which is responsive to the modulation of a component of the NLRP3 inflammasome pathway and/or which is responsive to the modulation of IL-1 beta and/or IL-18 levels that commonly lead to pathological inflammation.

### SUMMARY OF THE INVENTION

Accordingly, the present invention provides compounds that can be employed in the treatment, alleviation or prevention of a disease, disorder or an abnormality which is responsive to the modulation, in particular inhibition, of a component of the NLRP3 inflammasome pathway, or which is responsive to the modulation, in particular decrease, of IL-1 beta and/or IL-18 levels.

Various embodiments of the invention are described herein.

Within a certain aspect, provided herein is a compound of formula (I)

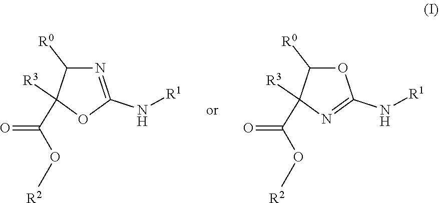

or stereoisomers, or racemic mixtures, or tautomers, or polymorphs, or pharmaceutically acceptable salts, or prodrugs, or hydrates, or solvates thereof;
wherein
R⁰ is H or C₁-C₃alkyl;
R² is independently selected from the group consisting of hydrogen, C₁-C₆alkyl, and C₃-C₆cycloalkyl;
R³ is independently selected from the group consisting of 5- or 6-membered heterocycloalkyl containing one to three heteroatoms, aryl and heteroaryl containing one or two heteroatoms. The heteroatoms in the 5- or 6-membered heterocycloalkyl containing one or two heteroatoms and the heteroaryl containing one or two heteroatoms are independently selected from N, S and O. In the 5- or 6-membered heterocycloalkyl containing one or two heteroatoms, and the heteroaryl having one or two heteroatoms at least one heteroatom is independently selected from O and S. The 5- or 6-membered heterocycloalkyl containing one or two heteroatoms, the aryl or the heteroaryl containing one or two heteroatoms can be optionally substituted with —C₁-C₆alkyl, —O—C₁-C₆alkyl, -Hal, or —C₁-C₆alkyl-OH;
R¹ is selected from the following ring systems

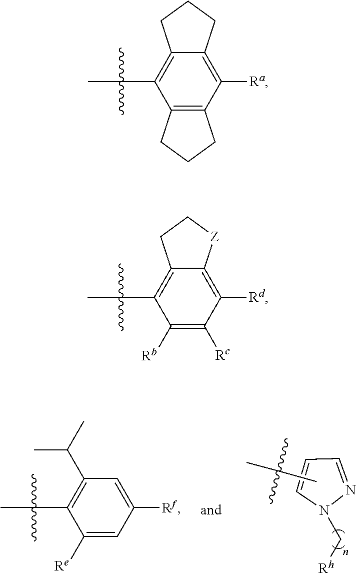

wherein

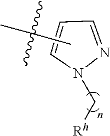

can be optionally substituted with C₁-C₆ alkyl at any available position;
Z is independently selected from the group consisting of CH₂ and O;
n is 0 or 1;
R^{a} is independently selected from the group consisting of C₁-C₃alkyl, C₁-C₃haloalkyl and phenyl;
R^{b} is heteroaryl optionally substituted with —C₁-C₆alkyl, —O—C₁-C₆alkyl, or —O—C₁-C₆haloalkyl;
R^{c} is independently selected from the group consisting of hydrogen and CH₃;
R^{d} is independently selected from the group consisting of hydrogen and halogen;
R^{e} independently selected from the group consisting of —C₁-C₆alkyl and heteroaryl, wherein heteroaryl is optionally substituted with —C₁-C₆alkyl, —C₁-C₆haloalkyl, —O—C₁-C₆alkyl, or —O—C₁-C₆haloalkyl;
R^{f} is independently selected from the group consisting of halogen and CF₃; and
R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl.

In all of the embodiments of R^{d}, R^{d} is preferably hydrogen or fluoro.

Within a preferred aspect, provided herein is a compound of formula (I)

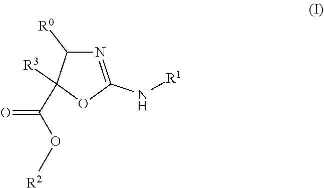

or a stereoisomer, a racemic mixture, a tautomer, a polymorph, a pharmaceutically acceptable salt, a prodrug, a hydrate, or a solvate thereof;
wherein
R⁰ is H or C₁-C₃alkyl;
R² is independently selected from the group consisting of hydrogen, and C₁-C₆alkyl, C₃-C₆cycloalkyl;
R³ is independently selected from the group consisting of 5- or 6-membered heterocycloalkyl containing one or two heteroatoms, aryl or heteroaryl containing one or two heteroatoms, wherein the heteroatoms are independently selected from N, S and O, and wherein at least one heteroatom is independently selected from O and S, and wherein the heterocycloalkyl, aryl or heteroaryl can be optionally substituted with —C₁-C₆alkyl, —O—C₁-C₆alkyl, -Hal, or —C₁-C₆alkyl-OH;
R¹ is selected from the following ring systems

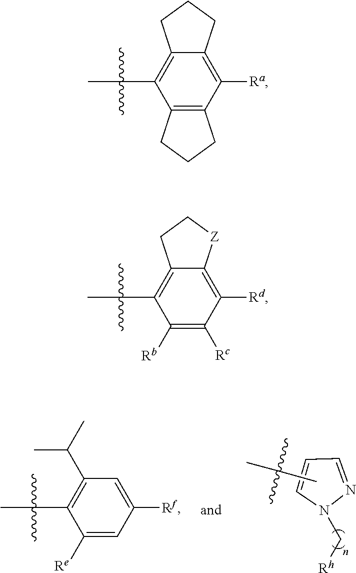

wherein

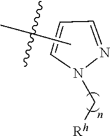

can be optionally substituted with —C₁-C₆alkyl at any available position;
Z is independently selected from the group consisting of CH₂ and O;
n is 0 or 1;
R^{a} is independently selected from the group consisting of —C₁-C₃alkyl, —C₁-C₃haloalkyl and phenyl;
R^{b} is heteroaryl optionally substituted with —C₁-C₆alkyl, —O—C₁-C₆alkyl, or —O—C₁-C₆haloalkyl;
R^{c} is independently selected from the group consisting of hydrogen or —CH₃;
R^{d} is independently selected from the group consisting of hydrogen or halogen;
R^{e} independently selected from the group consisting of —C₁-C₆alkyl, or heteroaryl, wherein heteroaryl is optionally substituted with —C₁-C₆alkyl, —C₁-C₆haloalkyl, —O—C₁-C₆alkyl, or —O—C₁-C₆haloalkyl;
R^{f} is independently selected from the group consisting of halogen and —CF₃; and
R^{h} is independently selected from the group consisting of —C₁-C₆alkyl and heteroaryl, wherein heteroaryl is optionally substituted with —C₁-C₆alkyl, —C₁-C₆haloalkyl, —O—C₁-C₆alkyl, or —O—C₁-C₆haloalkyl.

Within the present invention, the compound of formula (I) can have the formula

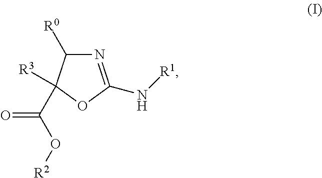

or be a stereoisomer, a racemic mixture, a tautomer, a polymorph, a pharmaceutically acceptable salt, a prodrug, a hydrate, or a solvate thereof;
wherein
X is independently selected from the group consisting of O and N;
Y is independently selected from the group consisting of N and O;
wherein one of X and Y is O and the other is N;
and wherein
R⁰, R², R³, R¹, Z, n, R^{a}, R^{b}, R^{c}, R^{d}, R^{e}, R^{f}, and R^{h} are as defined herein.

Within the present invention any reference to the compounds of formula (I) or (II), or the preferred embodiments thereof is intended to also refer to the stereoisomers, or racemic mixtures, or tautomers, or polymorphs, or pharmaceutically acceptable salts, or prodrugs, or hydrates, or solvates thereof.

Compounds of formula (I) or a stereoisomer, a racemic mixture, a tautomer, a polymorph, a pharmaceutically acceptable salt, a prodrug, a hydrate, or a solvate thereof, are suitable for the treatment, alleviation or prevention of a disease, disorder or an abnormality which is responsive to the modulation, in particular inhibition, of a component of the NLRP3 inflammasome pathway, or which is responsive to the modulation, in particular decrease, of IL-1 beta and/or IL-18 levels. In particular, the component of the inflammasome pathway is the NLRP3 inflammasome. Activation of the NLRP3 inflammasome pathway can trigger the formation of ASC specks, cleavage and activation of Caspase-1 and Caspase-8 and subsequent activation and release IL-1 beta, IL-18, gasdermin D cleavage and pore formation, pyroptosis, and release of IL-1alpha, IL-33, IL-17 and High-Mobility Group Box (HMGB) protein. The compounds of formula (I), or a stereoisomer, a racemic mixture, a tautomer, a polymorph, a pharmaceutically acceptable salt, a prodrug, a hydrate, or a solvate thereof, have the capability to modulate, in particular decrease, IL-1 beta and/or IL-18 levels.

The compounds of formula (I), or a stereoisomer, a racemic mixture, a tautomer, a polymorph, a pharmaceutically acceptable salt, a prodrug, a hydrate, or a solvate thereof, display high capability in modulating and, in particular inhibiting the activation of, a component of the NLRP3 inflammasome pathway, in particular wherein the component of the inflammasome pathway is the NLRP3 inflammasome. Due to their unique design features, these compounds display properties such as modulating or inhibiting the activation of the NLRP3 inflammasome pathway allowing them to be a successful medicament for the treatment, alleviation or prevention of diseases, disorders and abnormalities responsive to the modulation or inhibition of a component of the NLRP3 inflammasome pathway such as, for example, Alzheimer's disease, Parkinson's disease, CAPS, non-alcoholic fatty liver disease (NAFLD), non-alcoholic steatohepatitis (NASH) and gout.

In a further embodiment, the invention relates to a pharmaceutical composition comprising a compound of formula (I), or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient.

In another embodiment, the present invention refers to a compound of formula (I), or a stereoisomer, a racemic mixture, a tautomer, a polymorph, a pharmaceutically acceptable salt, a prodrug, a hydrate, or a solvate thereof, for use as a medicament.

Yet another embodiment, the present invention refers to a compound of formula (I), or a stereoisomer, a racemic mixture, a tautomer, a polymorph, a pharmaceutically acceptable salt, a prodrug, a hydrate, or a solvate thereof, for use in the treatment, alleviation or prevention of a disease, disorder, or abnormality which is responsive to the modulation, in particular inhibition of activation, of a component of the NLRP3 inflammasome pathway and/or which is responsive to the modulation, in particular decrease, of IL-1 beta and/or IL-18 levels.

A further embodiment is concerned with the use of the compound of formula (I), or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels.

In yet another embodiment, the present invention is directed to a method of treating, alleviating or preventing a disease, disorder or abnormality which is responsive to the modulation, in particular inhibition of activation, of a component of the NLRP3 inflammasome pathway or which is responsive to the modulation, in particular decrease, of IL-1 beta and/or IL-18 levels, the method comprising administering a therapeutically effective amount of a compound of formula (I), or a stereoisomer, a racemic mixture, a tautomer, a polymorph, a pharmaceutically acceptable salt, a prodrug, a hydrate, or a solvate thereof, to a subject in need thereof (e.g. a patient).

A pharmaceutical composition comprising a combination of a compound of formula (I), or a stereoisomer, a racemic mixture, a tautomer, a polymorph, a pharmaceutically acceptable salt, a prodrug, a hydrate, or a solvate thereof, and at least one further biologically active compound differing from the compound of formula (I), or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient, is also the subject-matter of the present invention.

In particular, the further biologically active compound can be one which is used for the treatment of a disease, disorder, or abnormality associated with a disease targeting different pathomechanism, e.g. an anti-amyloid beta antibody, anti-Tau antibody, amyloid beta small molecule inhibitor, Tau aggregation small molecule inhibitor, anti-alpha synuclein antibody or alpha-synuclein aggregation small molecule inhibitor, anti-TDP-43 antibody or anti-TDP-43 aggregation small molecule inhibitor, among others. When a compound of the invention is used in combination with a further biologically active compound, the dose of each compound may differ from the dose if the compound is used as monotherapy.

An additional embodiment relates to the use of the compound of formula (I), or a stereoisomer, a racemic mixture, a tautomer, a polymorph, a pharmaceutically acceptable salt, a prodrug, a hydrate, or a solvate thereof, as an analytical reference or an in vitro screening tool.

In another aspect the invention is further directed to a compound of formula (II):

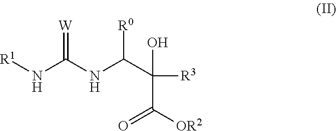

or a stereoisomer, a racemic mixture, a tautomer, a polymorph, a pharmaceutically acceptable salt, a prodrug, a hydrate, or a solvate thereof,
wherein
W is S or O;
R⁰, R¹, R² and R³ are as defined for formula (I).

In yet another embodiment, the present invention relates to methods of preparing a compound of formula (I), comprising the step of cyclization of a compound of formula (II).

The present invention is described hereinafter.

### DETAILED DESCRIPTION OF THE INVENTION

The present invention relates to compounds of formula (I), and to compounds of formula (II), including stereoisomers, racemic mixtures, tautomers, polymorphs, pharmaceutically acceptable salts, prodrugs, hydrates, or solvates thereof.

Any of the definitions of R⁰, R¹, R² and R³ which are given with respect to the compounds of formula (I) apply analogously to compounds of formula (II).

The present invention relates to compounds of formula (I) as defined below

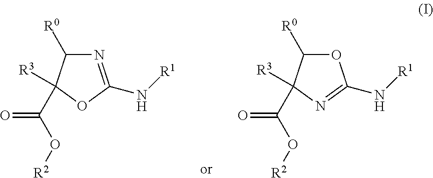

or a stereoisomer, a racemic mixture, a tautomer, a polymorph, a pharmaceutically acceptable salt, a prodrug, a hydrate, or a solvate thereof;
wherein
R⁰ is H or C₁-C₆alkyl;
R² is independently selected from the group consisting of hydrogen, C₁-C₆alkyl, and C₃-C₆cycloalkyl;
R³ is independently selected from the group consisting of 5- or 6-membered heterocycloalkyl containing one or two heteroatoms, aryl or heteroaryl containing one or two heteroatoms, wherein the heteroatom(s) is/are independently selected from N, O and S and at least one heteroatom is independently selected from O and S, and wherein the heterocycloalkyl, aryl or heteroaryl can be optionally substituted with —C₁-C₆alkyl, —O—C₁-C₆alkyl, -Hal, or —C₁-C₆alkyl-OH;
R¹ is selected from the following ring systems

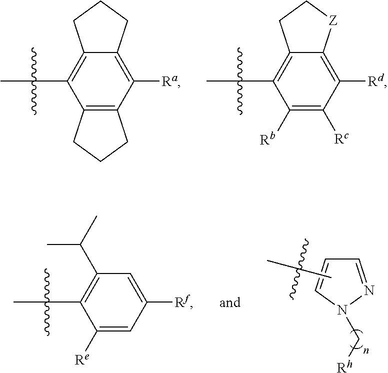

wherein

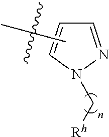

can be optionally substituted with C₁-C₆ alkyl at any available position;
Z is independently selected from the group consisting of CH₂ and O;
n is 0 or 1;
R^{a} is independently selected from the group consisting of C₁-C₃alkyl, C₁-C₃haloalkyl and phenyl;
R^{b} is heteroaryl optionally substituted with —C₁-C₆alkyl, —O—C₁-C₆alkyl, or —O—C₁-C₆haloalkyl;
R^{c} is independently selected from the group consisting of hydrogen and CH₃;
R^{d} is independently selected from the group consisting of hydrogen and halogen;
R^{e} independently selected from the group consisting of —C₁-C₆alkyl and heteroaryl, wherein heteroaryl is optionally substituted with —C₁-C₆alkyl, —C₁-C₆haloalkyl, —O—C₁-C₆alkyl, or —O—C₁-C₆haloalkyl;
R^{f} is independently selected from the group consisting of halogen and CF₃; and
R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl (e.g., CF₃).

Within a preferred aspect, the invention is related to compounds of formula (I)

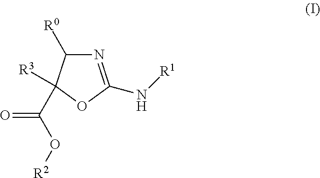

or a stereoisomer, a racemic mixture, a tautomer, a polymorph, a pharmaceutically acceptable salt, a prodrug, a hydrate, or a solvate thereof;
wherein
R⁰ is H or C₁-C₃alkyl;
R² is independently selected from the group consisting of hydrogen, C₁-C₆alkyl, C₃-C₆cycloalkyl;
R³ is independently selected from the group consisting of 5- or 6-membered heterocycloalkyl containing one or two heteroatoms, aryl or heteroaryl containing one or two heteroatoms, wherein the heteroatoms are independently selected from N, S and O, and wherein at least one heteroatom is independently selected from O and S, and wherein the heterocycloalkyl, aryl or heteroaryl can be optionally substituted with —C₁-C₆alkyl, —O—C₁-C₆alkyl, -Hal, or —C₁-C₆alkyl-OH;
R¹ is selected from the following ring systems

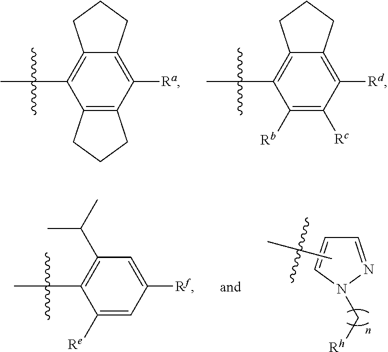

wherein

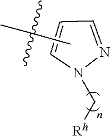

can be optionally substituted with —C₁-C₆alkyl at any available position;
Z is independently selected from the group consisting of CH₂ and O;
n is 0 or 1;
R^{a} is independently selected from the group consisting of —C₁-C₃alkyl, —C₁-C₃haloalkyl and phenyl;
R^{b} is heteroaryl optionally substituted with —C₁-C₆alkyl, —O—C₁-C₆alkyl, or —O—C₁-C₆haloalkyl;
R^{e} is independently selected from the group consisting of hydrogen or —CH₃;
R^{d} is independently selected from the group consisting of hydrogen or halogen;
R^{e} independently selected from the group consisting of —C₁-C₆alkyl, or heteroaryl, wherein heteroaryl is optionally substituted with —C₁-C₆alkyl, —C₁-C₆haloalkyl, —O—C₁-C₆alkyl, or —O—C₁-C₆haloalkyl;
R^{f} is independently selected from the group consisting of halogen and —CF₃; and
R^{h} is independently selected from the group consisting of —C₁-C₆alkyl and heteroaryl, wherein heteroaryl is optionally substituted with —C₁-C₆alkyl, —C₁-C₆haloalkyl, —O—C₁-C₆alkyl, or —O—C₁-C₆haloalkyl.

In a preferred embodiment, R⁰ is H or C₁-C₃alkyl. Preferably, R⁰ is H, methyl or ethyl. More preferably, R⁰ is H. In one embodiment R⁰ is ethyl.

In one embodiment, R¹ is

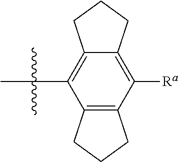

wherein R^{a} is independently selected from C₁-C₃alkyl, C₁-C₃haloalkyl and phenyl. In one embodiment R^{a} is C₁-C₃alkyl, preferably methyl or ethyl. More preferably R^{a} is methyl. In another embodiment R^{a} is C₁-C₆haloalkyl, preferably CF₃. In another embodiment R^{a} is phenyl.

In another embodiment, R¹ is

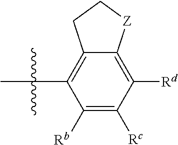

wherein
Z is independently selected from the group consisting of CH₂ and O. In one embodiment Z is CH₂. In another embodiment Z is O.
R^{b} is heteroaryl optionally substituted with —C₁-C₆alkyl, —O—C₁-C₆alkyl, or —O—C₁-C₆haloalkyl;
R^{c} is independently selected from the group consisting of hydrogen and CH₃; and
R^{d} is independently selected from the group consisting of hydrogen and halogen.

In another embodiment. R¹ is

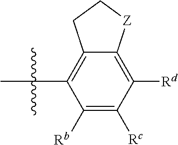

wherein
Z is independently selected from the group consisting of CH₂ and O. In one embodiment Z is CH₂. In another embodiment Z is O.
R^{b} is heteroaryl containing one or two heteroatoms independently selected from O, N and S, and preferably at least one heteroatom is N. The heteroaryl is optionally substituted with —C₁-C₆alkyl, —O—C₁-C₆alkyl, or —O—C₁-C₆haloalkyl;
R^{c} is independently selected from the group consisting of hydrogen and CH₃; and
R^{d} is independently selected from the group consisting of hydrogen and halogen.

In another embodiment, R¹ is

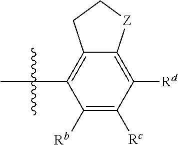

wherein
Z is independently selected from the group consisting of CH₂ and O. In one embodiment Z is CH₂. In another embodiment Z is O.
R^{b} is heteroaryl containing one or two heteroatoms, wherein said one or two heteroatom(s) is/are N, preferably R^{b} is pyridyl; the heteroaryl is optionally substituted with —C₁-C₆alkyl, —O—C₁-C₆alkyl, or —O—C₁-C₆haloalkyl;
R^{c} is independently selected from the group consisting of hydrogen and CH₃; and
R^{d} is independently selected from the group consisting of hydrogen and halogen.

In another embodiment, R¹ is

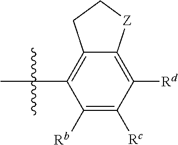

wherein
Z is independently selected from the group consisting of CH₂ and O. In one embodiment Z is CH₂. In another embodiment Z is O.
R^{b} is heteroaryl containing one or two heteroatoms, wherein said one or two heteroatom(s) is/are N, preferably R^{b} is pyridyl, substituted with —C₁-C₆alkyl, —O—C₁-C₆alkyl, or —O—C₁-C₆haloalkyl. In one preferred embodiment R^{b} is pyridyl substituted with —C₁-C₆alkyl, preferably —C₁-C₃alkyl. In another preferred embodiment R^{b} is pyridyl substituted with —O—C₁-C₆alkyl, preferably —O—C₁-C₃alkyl.
R^{c} is independently selected from the group consisting of hydrogen and CH₃. In one preferred embodiment R^{c} is hydrogen. In another embodiment R^{c} is CH₃.
R^{d} is independently selected from the group consisting of hydrogen and halogen. In one preferred embodiment R^{d} is hydrogen. In another embodiment R^{d} is halogen.

In another embodiment, R¹ is

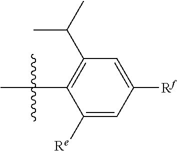

wherein
R^{e} is independently selected from —C₁-C₆alkyl, or heteroaryl, wherein heteroaryl is optionally substituted with —C₁-C₆alkyl, —C₁-C₆haloalkyl, —O—C₁-C₆alkyl, or —O—C₁-C₆haloalkyl;
R^{f} is independently selected from halogen or CF₃. In one preferred embodiment R^{f} is F.

In another embodiment, R¹ is

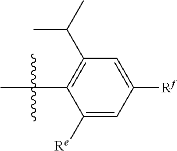

wherein
R^{e} is selected from branched C₃-C₆alkyl, preferably isopropyl or isobutyl; or heteroaryl having one or two heteroatoms independently selected from O, N and S, and preferably at least one heteroatom is N, wherein the heteroaryl is optionally substituted with —C₁-C₆alkyl, —C₁-C₆haloalkyl, —O—C₁-C₆alkyl, or —O—C₁-C₆haloalkyl;
R^{f} is independently selected from halogen or CF₃. In one embodiment R^{f} is F.

In one preferred embodiment R^{e} is isopropyl.

In another embodiment R^{e} is heteroaryl having one or two heteroatoms independently selected from O, N and S, and preferably at least one heteroatom is N. The heteroaryl is optionally substituted with —C₁-C₆alkyl, —C₁-C₆haloalkyl, —O—C₁-C₆alkyl, or —O—C₁-C₆haloalkyl. In one embodiment R^{e} is heteroaryl having one or two heteroatoms, wherein said one or two heteroatom(s) is/are N, preferably R^{e} is pyridyl. In another embodiment R^{e} is heteroaryl having one or two heteroatoms, wherein said one or two heteroatom(s) is/are N, preferably R^{e} is pyridyl, substituted with —C₁-C₆alkyl, —C₁-C₆haloalkyl, —O—C₁-C₆alkyl, or —O—C₁-C₆haloalkyl. In one preferred embodiment R^{e} is pyridyl substituted with —C₁-C₆alkyl, preferably —C₁-C₃alkyl. In another preferred embodiment R^{e} is pyridyl substituted with —O—C₁-C₆alkyl, preferably —O—C₁-C₃alkyl.

In any of the embodiments of R^{e}, the substituents of the heteroaryl are preferably —C₁-C₆alkyl, —O—C₁-C₆alkyl, or —O—C₁-C₆haloalkyl.

In another embodiment, R¹ is

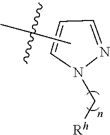

optionally substituted with C₁-C₆ alkyl at any available position,
and wherein R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl, (e.g., CF₃);
n is 0 or 1.

In one embodiment, R¹ is

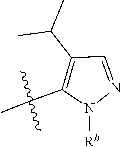

wherein
R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl (e.g., CF₃), preferably R^{h} is heteroaryl optionally substituted with —C₁-C₆alkyl, —O—C₁-C₆alkyl, —C₁-C₆haloalkyl, or —O—C₁-C₆haloalkyl. In one embodiment R^{h} is heteroaryl having one or two heteroatoms independently selected from O, N and S, and preferably at least one heteroatom is N, wherein the heteroaryl is optionally substituted with —C₁-C₆alkyl, —C₁-C₆haloalkyl, —O—C₁-C₆alkyl, or —O—C₁-C₆haloalkyl. In one embodiment R^{h} is heteroaryl having one or two heteroatoms, wherein the one or two heteroatom(s) is/are N, preferably R^{h} is pyridyl. In another embodiment R^{h} is heteroaryl having one or two heteroatoms, wherein the one or two heteroatom(s) is/are N, preferably R^{h} is pyridyl, substituted with —C₁-C₆alkyl, —C₁-C₆haloalkyl, —O—C₁-C₆alkyl, or —O—C₁-C₆haloalkyl. In one preferred embodiment R^{h} is pyridyl substituted with —C₁-C₆alkyl, preferably —C₁-C₃alkyl. In another preferred embodiment R^{h} is pyridyl substituted with —O—C₁-C₆alkyl, preferably —O—C₁-C₃alkyl.

In one embodiment, R¹ is

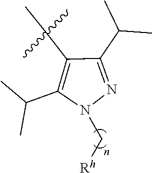

wherein n is 0 or 1, and R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl (e.g., CF₃). In one embodiment R^{h} is CF₃. In another embodiment R^{h} is heteroaryl optionally substituted with —C₁-C₆alkyl, —C₁-C₆haloalkyl, —O—C₁-C₆alkyl, or —O—C₁-C₆haloalkyl. In one embodiment R^{h} is heteroaryl having one or two heteroatoms independently selected from O, N and S, and preferably at least one heteroatom is N, wherein the heteroaryl is optionally substituted with —C₁-C₆alkyl, —C₁-C₆haloalkyl, —O—C₁-C₆alkyl, or —O—C₁-C₆haloalkyl. In one embodiment R^{h} is heteroaryl having one or two heteroatoms, wherein the one or two heteroatom(s) is/are N, preferably R^{h} is pyridyl. In another embodiment R^{h} is heteroaryl having one or two heteroatoms, wherein the one or two heteroatom(s) is/are N, preferably R^{h} is pyridyl, substituted with —C₁-C₆alkyl, —C₁-C₆haloalkyl, —O—C₁-C₆alkyl, or —O—C₁-C₆haloalkyl. In one preferred embodiment R^{h} is pyridyl substituted with —C₁-C₆alkyl, preferably —C₁-C₆alkyl. In another preferred embodiment R^{h} is pyridyl substituted with —O—C₁-C₆alkyl, preferably —O—C₁-C₆alkyl.

In any of the embodiments herein, the optional substituents of the heteroaryl in R^{h} are preferably selected from —C₁-C₆alkyl, CF₃, —O—C₁-C₆alkyl, or —O—C₁-C₆haloalkyl.

In another embodiment, R² is independently selected from the group consisting of hydrogen, —C₁-C₆alkyl, and C₃-C₆cycloalkyl.

Preferably, R² is independently selected from the group consisting of hydrogen, C₁-C₆alkyl, and C₅-C₆cycloalkyl. In one preferred embodiment, R² is hydrogen. In another preferred embodiment, R² is C₁-C₆alkyl, wherein R² is preferably selected from the group consisting of methyl, ethyl, propyl, isopropyl, butyl, tert-butyl, pentyl, or hexyl. In yet another embodiment, R² is C₅-C₆cycloalkyl, preferably, R² is cyclopentane.

More preferably, R² is independently selected from the group consisting of hydrogen, C₁-C₃alkyl, and C₅-C₆cycloalkyl. Even more preferably, R² is independently selected from the group consisting of hydrogen, methyl, ethyl, propyl, isopropyl or cyclopentane. Even more preferably, R² is independently selected from the group consisting of hydrogen, ethyl, isopropyl or cyclopentane. In one preferred embodiment, R² is ethyl or H. In one preferred embodiment, R² is ethyl.

In any of the embodiments of R³, the heteroatom in the heterocycloalkyl and the heteroaryl, respectively, is independently selected from N, O and S.

In one embodiment, R³ is independently selected from the group consisting of 5- or 6-membered heterocycloalkyl containing one or two heteroatoms, aryl or heteroaryl containing one or two heteroatoms. The heteroatom(s) in the heterocycloalkyl and the heteroaryl, respectively, is/are independently selected from N, O and S. At least one heteroatom in the heterocycloalkyl and the heteroaryl, respectively, is independently selected from O and S. The heterocycloalkyl, aryl or heteroaryl can be optionally substituted with —C₁-C₆alkyl, —O—C₁-C₆alkyl, -Hal, or —C₁-C₆alkyl-OH.

In another embodiment, R³ is independently selected from the group consisting of 5- or 6-membered heterocycloalkyl containing one or two heteroatoms, wherein at least one heteroatom is O, aryl and 5- or 6-membered heteroaryl containing one or two heteroatoms, wherein at least one heteroatom is independently selected from O and S. The heteroatom in the heterocycloalkyl and the heteroaryl, respectively, is independently selected from N, O and S. The heterocycloalkyl, aryl or heteroaryl can be optionally substituted with —C₁-C₆alkyl, —O—C₁-C₆alkyl, -Hal, or —C₁-C₆alkyl-OH.

In one embodiment, R³ is aryl, optionally substituted with —C₁-C₆alkyl, —O—C₁-C₆alkyl, -Hal, or —C₁-C₆alkyl-OH. In a particular embodiment, the aryl group is optionally substituted with —O—C₁-C₆alkyl, or -Hal. In a particular embodiment, the aryl group is optionally substituted with —O—C₁alkyl, or -Hal.

In the embodiments of R³, the halogen (-Hal) is preferably chloro.

More preferably, R³ is independently selected from the group consisting of 5- or 6-membered heterocycloalkyl containing one or two heteroatoms wherein at least one heteroatom is O, C₅-C₆ aryl, or 5- or 6-membered heteroaryl containing one or two heteroatoms wherein at least one heteroatom is independently selected from O and S. The heteroatom(s) in the heterocycloalkyl and the heteroaryl, respectively, is/are independently selected from N, O and S. The 5- or 6-membered heterocycloalkyl, aryl or 5- or 6-membered heteroaryl can be optionally substituted with —C₁-C₆alkyl, —O—C₁-C₆alkyl, -Hal, or —C₁-C₆alkyl-OH.

In one preferred embodiment, R³ is 5- or 6-membered heterocycloalkyl containing one heteroatom, wherein the heteroatom is O; and the heterocycloalkyl is optionally substituted with —C₁-C₆alkyl, —O—C₁-C₆alkyl, -Hal, or —C₁-C₆alkyl-OH.

In another preferred embodiment, R³ is 5- or 6-membered heteroaryl containing one or two heteroatoms independently selected from N, O and S, wherein at least one heteroatom is independently selected from O and S, and the heteroaryl is optionally substituted with one substituent selected from —C₁-C₆alkyl, —O—C₁-C₆alkyl, -Hal, or —C₁-C₆alkyl-OH.

In another preferred embodiment, R³ is C₅-C₆ heteroaryl containing two heteroatoms, wherein at least one heteroatom is independently selected from O and S, and the second heteroatom is N, and the heteroaryl is optionally substituted with one substituent selected from —C₁-C₆alkyl, —O—C₁-C₆alkyl, -Hal, or —C₁-C₆alkyl-OH.

In another embodiment, R³ is selected from the following:

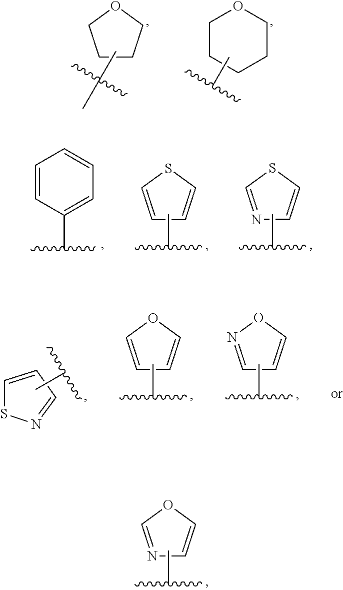

wherein each of them can be optionally substituted with —C₁-C₆alkyl, —O—C₁-C₆alkyl, -Hal, or —C₁-C₆alkyl-OH.

More preferably, R³ is selected from the following:

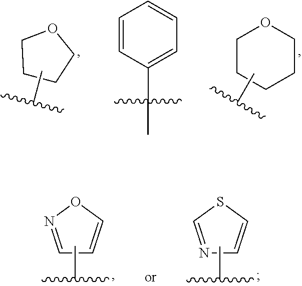

wherein each of them can be optionally substituted with —C₁-C₆alkyl, —O—C₁-C₆alkyl, -Hal, or —C₁-C₆alkyl-OH.

In another preferred embodiment, R³ is selected from the following:

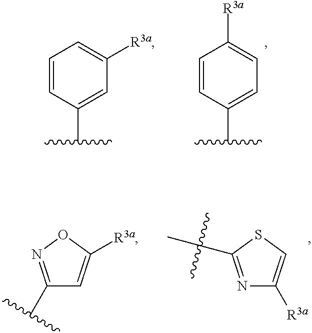

wherein R^{3a} is selected from hydrogen, halogen, —O—C₁-C₆alkyl, —C₁-C₆ alkyl-OH, and —C₁-C₆alkyl.

More preferably, R^{3a} is selected from hydrogen, chloro, methoxy, methyl, isopropyl, and

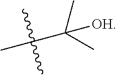 [SMILES: *C(C)(C)O]

In R³, C₁-C₆alkyl is preferably methyl, ethyl, isopropyl, propyl, isobutyl or butyl and can be optionally substituted with —OH such as

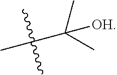 [SMILES: *C(C)(C)O]

In another embodiment, R² is hydrogen or C₁-C₆alkyl, and R³ is
a)

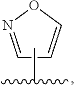

preferably

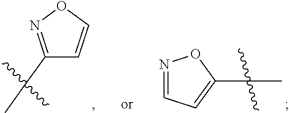 [SMILES: *c1ccon1 ; *c1ccno1]

b)

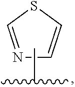

preferably

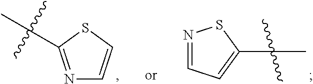 [SMILES: *c1nccs1 ; *c1ccns1]

c)

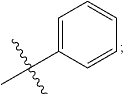 [SMILES: *c1ccccc1]

d)

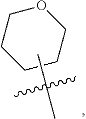

preferably

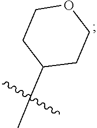 [SMILES: *C1CCOCC1]

e)

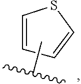

preferably

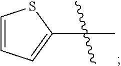 [SMILES: *c1cccs1 ; ";"]

f)

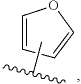

preferably

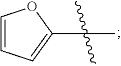 [SMILES: *c1ccco1 ; ";"]

g)

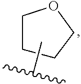

preferably

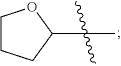 [SMILES: *C1CCCO1 ; ";"]

In another embodiment, R² is hydrogen or C₁-C₆alkyl, and R³ is
a)

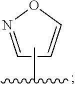

preferably

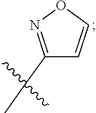 [SMILES: *c1ccon1]

b)

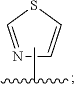

preferably

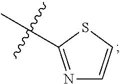 [SMILES: *c1nccs1]

c)

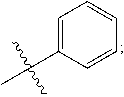 [SMILES: *c1ccccc1]

d)

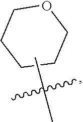

preferably

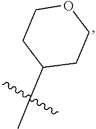 [SMILES: *C1CCOCC1]

In each of the above embodiments, R³ can be optionally substituted with halogen, —O—C₁-C₆alkyl, —C₁-C₆alkyl-OH, or C₁-C₆alkyl. More preferably, R³ can be optionally substituted with chloro, methoxy, methyl or isopropyl or

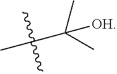 [SMILES: *C(C)(C)O]

In R³, C₁-C₆alkyl is preferably methyl, ethyl, isopropyl, propyl, isobutyl or butyl and can be optionally substituted with —OH such as in

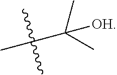 [SMILES: *C(C)(C)O]

Preferably, R² is C₁-C₆alkyl, wherein R² is preferably selected from the group consisting of methyl, ethyl, propyl, isopropyl, butyl, tert-butyl, pentyl and hexyl. More preferably, R² is C₁-C₃alkyl. More preferably, R² is independently selected from the group consisting of methyl, ethyl, propyl and isopropyl. Even more preferably, R² is independently selected from the group consisting of ethyl and isopropyl, most preferably R² is ethyl.

In a further preferred embodiment, R² is hydrogen.

In a further embodiment, the present invention relates to compounds of formula (I)

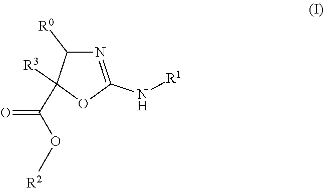

or a stereoisomer, a racemic mixture, a tautomer, a polymorph, a pharmaceutically acceptable salt, a prodrug, a hydrate, or a solvate thereof;
wherein R¹, R² are R³ are as defined above and wherein R⁰ is H.

In a further embodiment, the present invention relates to compounds of formula (I)

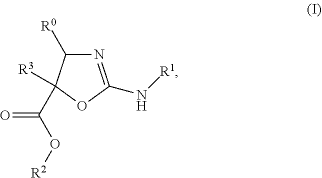

or a stereoisomer, a racemic mixture, a tautomer, a polymorph, a pharmaceutically acceptable salt, a prodrug, a hydrate, or a solvate thereof
wherein
R⁰ is H;
R¹ is as defined above;
R² is H or C₁-C₆ alkyl, such as C₁-C₆ alkyl; and
R³ is independently selected from the group consisting of

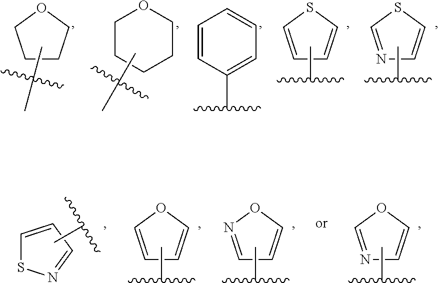

each of them can be optionally substituted as defined above.

In a further embodiment, the present invention relates to the following compounds of formula (I)

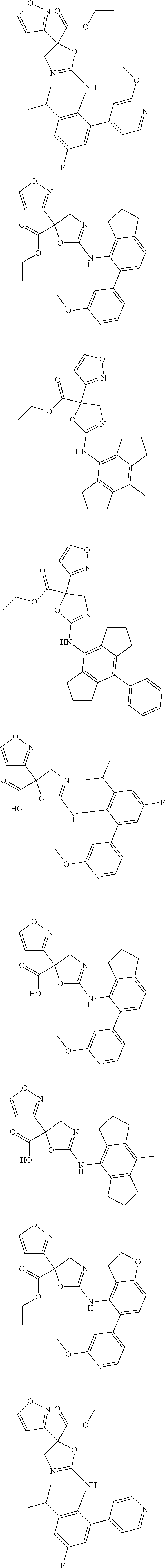

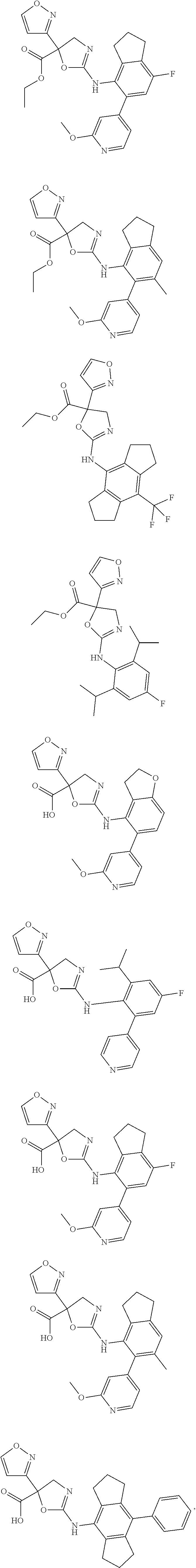

or a stereoisomer, a racemic mixture, a tautomer, a polymorph, a pharmaceutically acceptable salt, a prodrug, a hydrate, or a solvate thereof.

In one embodiment, the present invention further relates to a compound of formula (II) as defined below

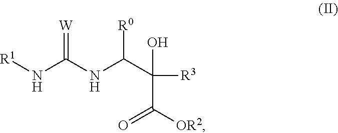

or stereoisomers, racemic mixtures, tautomers, polymorphs, pharmaceutically acceptable salts, prodrugs, hydrates, or solvates thereof;
wherein
W is S or O;
R⁰, R¹, R² and R³ are as defined above for formula (I).

In one embodiment of formula (II)
W is S or O;
R⁰ is H or C₁-C₃alkyl;
R² is independently selected from the group consisting of hydrogen, C₁-C₆alkyl and C₃-C₆cycloalkyl; and
R³ is independently selected from the group consisting of 5- or 6-membered heterocycloalkyl containing one or two heteroatoms, aryl and heteroaryl containing one or two heteroatoms. The heteroatom(s) in the heterocycloalkyl and heteroaryl, respectively, is/are independently selected from N, O and S and at least one heteroatom is independently selected from O and S. The 5- or 6-membered heterocycloalkyl containing one or two heteroatoms, aryl or heteroaryl containing one or two heteroatoms can be optionally substituted with —C₁-C₆alkyl, —O—C₁-C₆alkyl, -Hal, or —C₁-C₆alkyl-OH;
R¹ is selected from the following ring systems

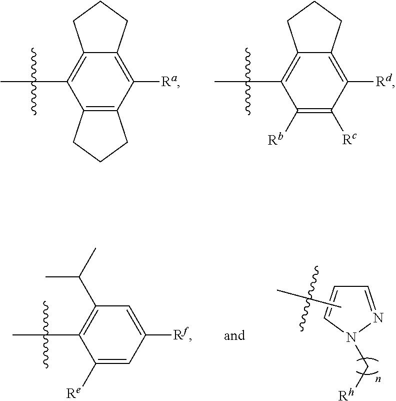

wherein

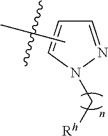

can be optionally substituted with C₁-C₆ alkyl at any available position;
Z is independently selected from the group consisting of CH₂ and O;
n is 0 or 1;
R^{a} is independently selected from the group consisting of C₁-C₃alkyl, C₁-C₃haloalkyl and phenyl;
R^{b} is heteroaryl optionally substituted with —C₁-C₆alkyl, —O—C₁-C₆alkyl, and —O—C₁-C₆haloalkyl;
R^{c} is independently selected from the group consisting of hydrogen and CH₃;
R^{d} is independently selected from the group consisting of hydrogen and halogen;
R^{e} independently selected from the group consisting of —C₁-C₆alkyl, and heteroaryl, wherein the heteroaryl is optionally substituted with —C₁-C₆alkyl, —C₁-C₆haloalkyl, —O—C₁-C₆alkyl, or —O—C₁-C₆haloalkyl;
R^{f} is independently selected from the group consisting of halogen and CF₃;
R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl.

In one embodiment, the present invention relates to a compound of formula (II), wherein W is O. In another embodiment, the present invention relates to a compound of formula (II), W is S. In a preferred embodiment, W is S.

In one embodiment, R⁰ is H or C₁-C₃alkyl. Preferably, R⁰ is H, methyl or ethyl. More preferably, R⁰ is H. In one embodiment, R⁰ is ethyl.

In one embodiment, R¹ is

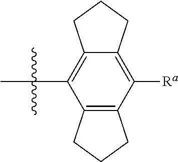

wherein R^{a} is independently selected from the group consisting of C₁-C₃alkyl, C₁-C₃haloalkyl and phenyl. In one embodiment, R^{a} is C₁-C₃alkyl, preferably methyl or ethyl. More preferably, R^{a} is methyl. In another embodiment, R^{a} is C₁-C₆haloalkyl, preferably CF₃. In another embodiment, R^{a} is phenyl.

In another embodiment, R¹ is

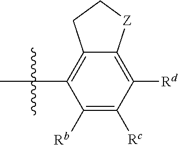

wherein
Z is independently selected from the group consisting of CH₂ and O. In one embodiment, Z is CH₂. In another embodiment, Z is O.
R^{b} is heteroaryl optionally substituted with —C₁-C₆alkyl, —O—C₁-C₆alkyl, or —O—C₁-C₆haloalkyl;
R^{c} is independently selected from the group consisting of hydrogen and CH₃;
R^{d} is independently selected from the group consisting of hydrogen and halogen.

In another embodiment, R¹ is

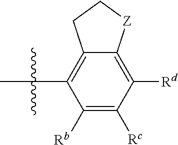

wherein
Z is independently selected from the group consisting of CH₂ and O. In one embodiment Z is CH₂. In another embodiment, Z is O.
R^{b} is heteroaryl having one or two heteroatoms independently selected from O, N and S, and preferably at least one heteroatom is N, the heteroaryl can be optionally substituted with —C₁-C₆alkyl, —O—C₁-C₆alkyl, or —O—C₁-C₆haloalkyl;
R^{c} is independently selected from the group consisting of hydrogen and CH₃;
R^{d} is independently selected from the group consisting of hydrogen and halogen.

In another embodiment, R¹ is

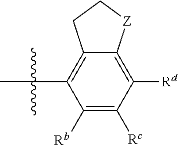

wherein
Z is independently selected from the group consisting of CH₂ and O. In one embodiment Z is CH₂. In another embodiment, Z is O,
R^{b} is heteroaryl having one or two heteroatoms wherein said one or two heteroatom(s) is/are N, preferably R^{b} is pyridyl; the heteroaryl can be optionally substituted with —C₁-C₆alkyl, —O—C₁-C₆alkyl, or —O—C₁-C₆haloalkyl;
R^{c} is independently selected from the group consisting of hydrogen and CH₃;
R^{d} is independently selected from the group consisting of hydrogen and halogen; In another embodiment, R¹ is

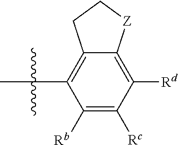

wherein
Z is independently selected from the group consisting of CH₂ and O. In one embodiment, Z is CH₂. In another embodiment, Z is O.
R^{b} is heteroaryl having one or two heteroatoms wherein said one or two heteroatom(s) is/are N, preferably R^{b} is pyridyl, substituted with —C₁-C₆alkyl, —O—C₁-C₆alkyl, or —O—C₁-C₆haloalkyl. In one preferred embodiment, R^{b} is pyridyl substituted with —C₁-C₆alkyl, preferably —C₁-C₃alkyl. In another preferred embodiment, R^{b} is pyridyl substituted with —O—C₁-C₆alkyl, preferably —O—C₁-C₃alkyl.
R^{c} is independently selected from the group consisting of hydrogen and CH₃. In one preferred embodiment, R^{c} is hydrogen. In another embodiment, R^{c} is CH₃.
R^{d} is independently selected from the group consisting of hydrogen and halogen. In one preferred embodiment, R^{d} is hydrogen. In another embodiment, R^{d} is halogen.

In another embodiment, R¹ is

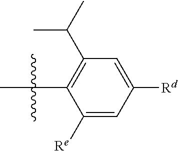

wherein
R^{e} is independently selected from the group consisting of —C₁-C₆alkyl, and heteroaryl, wherein heteroaryl is optionally substituted with —C₁-C₆alkyl, —C₁-C₆haloalkyl, —O—C₁-C₆alkyl, or —O—C₁-C₆haloalkyl;
R^{f} is independently selected from the group consisting of halogen and CF₃. In one preferred embodiment, R^{f} is F.

In another embodiment, R¹ is

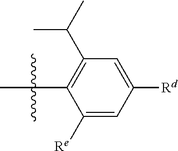

wherein
R^{e} is independently selected from the group consisting of branched C₃-C₆alkyl, preferably isopropyl or isobutyl; or heteroaryl having one or two heteroatoms independently selected from O, N and S, and preferably at least one heteroatom is N, the heteroaryl can be optionally substituted with —C₁-C₆alkyl, —C₁-C₆haloalkyl, —O—C₁-C₆alkyl, or —O—C₁-C₆haloalkyl;
R^{f} is independently selected from the group consisting of halogen or CF₃. In one embodiment, R^{f} is F.

In one preferred embodiment, R^{e} is isopropyl.

In another embodiment, R^{e} is heteroaryl having one or two heteroatoms independently selected from O, N and S, and preferably at least one heteroatom is N, wherein the heteroaryl is optionally substituted with —C₁-C₆alkyl, —C₁-C₆haloalkyl, —O—C₁-C₆alkyl, or —O—C₁-C₆haloalkyl. In one embodiment, R^{e} is heteroaryl having one or two heteroatoms, wherein said one or two heteroatom(s) is/are N, preferably R^{e} is pyridyl. In another embodiment, R^{e} is heteroaryl having one or two heteroatoms, wherein said one or two heteroatom(s) is/are N, preferably R^{e} is pyridyl, wherein the heteroaryl is substituted with —C₁-C₆alkyl, —C₁-C₆haloalkyl, —O—C₁-C₆alkyl, or —O—C₁-C₆haloalkyl. In one preferred embodiment, R^{e} is pyridyl substituted with —C₁-C₆alkyl, preferably —C₁-C₃alkyl. In another preferred embodiment, R^{e} is pyridyl substituted with —O—C₁-C₆alkyl, preferably —O—C₁-C₃alkyl.

In another embodiment, R¹ is

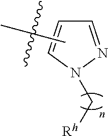

optionally substituted with C₁-C₆ alkyl at any available position,
and wherein R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl, e.g. CF₃;
n is 0 or 1.

In one embodiment. R¹ is

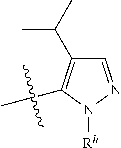

wherein R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl (e.g., CF₃), preferably R^{h} is heteroaryl optionally substituted with —C₁-C₆alkyl, —C₁-C₆haloalkyl, —O—C₁-C₆alkyl, or —O—C₁-C₆haloalkyl. In one embodiment, R^{h} is heteroaryl having one or two heteroatoms independently selected from O, N and S, and preferably at least one heteroatom is N, the heteroaryl can be optionally substituted with —C₁-C₆alkyl, —C₁-C₆haloalkyl, —O—C₁-C₆alkyl, or —O—C₁-C₆haloalkyl. In one embodiment, R^{h} is heteroaryl having one or two heteroatoms, wherein said one or two heteroatom(s) is/are N, preferably R^{h} is pyridyl. In another embodiment, R^{h} is heteroaryl having one or two heteroatoms, wherein said one or two heteroatom(s) is/are N, preferably R^{h} is pyridyl, substituted with —C₁-C₆alkyl, —C₁-C₆haloalkyl, —O—C₁-C₆alkyl, or —O—C₁-C₆haloalkyl. In one preferred embodiment, R^{h} is pyridyl substituted with —C₁-C₆alkyl, preferably —C₁-C₃alkyl. In another preferred embodiment, R^{h} is pyridyl substituted with —O—C₁-C₆alkyl, preferably —O—C₁-C₃alkyl.

In one embodiment, R¹ is

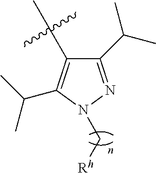

wherein n is 0 or 1, and
R^{h} is independently selected from the group consisting of —C₁-C₆alkyl and heteroaryl, wherein the heteroaryl is optionally substituted with —C₁-C₆alkyl, —C₁-C₆haloalkyl, —O—C₁-C₆alkyl, or —O—C₁-C₆haloalkyl (e.g., CF₃). In one embodiment R^{h} is CF₃. In another embodiment, R^{h} is heteroaryl optionally substituted with —C₁-C₆alkyl, —C₁-C₆haloalkyl, —O—C₁-C₆alkyl, or —O—C₁-C₆haloalkyl. In one embodiment, R^{h} is heteroaryl having one or two heteroatoms independently selected from O, N and S, and preferably at least one heteroatom is N, wherein the heteroaryl is optionally substituted with —C₁-C₆alkyl, —C₁-C₆haloalkyl, —O—C₁-C₆alkyl, or —O—C₁-C₆haloalkyl. In one embodiment, R^{h} is heteroaryl having one or two heteroatoms, wherein said one or two heteroatom(s) is/are N, preferably R^{h} is pyridyl. In another embodiment, R^{h} is heteroaryl having one or two heteroatoms, wherein said one or two heteroatom(s) is/are N, preferably R^{h} is pyridyl, substituted with —C₁-C₆alkyl, —C₁-C₆haloalkyl, —O—C₁-C₆alkyl, or —O—C₁-C₆haloalkyl. In one preferred embodiment, R^{h} is pyridyl substituted with —C₁-C₆alkyl, preferably —C₁-C₃alkyl. In another preferred embodiment, R^{h} is pyridyl substituted with —O—C₁-C₆alkyl, preferably —O—C₁-C₃alkyl.

In another embodiment, R² is independently selected from the group consisting of hydrogen, C₁-C₆alkyl, and C₃-C₆cycloalkyl.

Preferably, R² is independently selected from the group consisting of hydrogen, C₁-C₆alkyl, and C₅-C₆cycloalkyl. In one preferred embodiment, R² is hydrogen. In another preferred embodiment, R² is C₁-C₆alkyl, wherein R² is preferably selected from the group consisting of methyl, ethyl, propyl, isopropyl, butyl, tert-butyl, pentyl and hexyl. In yet another embodiment, R² is C₅-C₆cycloalkyl, preferably, R² is cyclopentane.

More preferably, R² is independently selected from the group consisting of hydrogen, C₁-C₃alkyl, and C₅-C₆cycloalkyl. Even more preferably, R² is independently selected from the group consisting of hydrogen, methyl, ethyl, propyl, isopropyl and cyclopentane. Even more preferably, R² is independently selected from the group consisting of hydrogen, ethyl, isopropyl and cyclopentane. In one preferred embodiment, R² is ethyl or hydrogen. In one preferred embodiment, R² is ethyl.

In one embodiment, R³ is independently selected from the group consisting of 5- or 6-membered heterocycloalkyl containing one or two heteroatoms, aryl and heteroaryl containing one or two heteroatoms. The heteroatom(s) in heterocycloalkyl and heteroaryl, respectively, is/are independently selected from N, O and S. At least one heteroatom is independently selected from O and S. The heterocycloalkyl, aryl or heteroaryl can be optionally substituted with —C₁-C₆alkyl, —O—C₁-C₆alkyl, -Hal, or —C₁-C₆alkyl-OH.

In another embodiment, R³ is independently selected from the group consisting of 5- or 6-membered heterocycloalkyl containing one or two heteroatoms, wherein at least one heteroatom is O, aryl and 5- or 6-membered heteroaryl containing one or two heteroatoms, wherein at least one heteroatom is independently selected from O and S. The heteroatom(s) in heterocycloalkyl and heteroaryl, respectively, is/are independently selected from N, O and S. The heterocycloalkyl, aryl or heteroaryl can be optionally substituted with —C₁-C₆alkyl, —O—C₁-C₆alkyl, -Hal, or —C₁-C₆alkyl-OH.

In one embodiment, R³ is aryl, optionally substituted with —C₁-C₆alkyl, —O—C₁-C₆alkyl, -Hal, or —C₁-C₆alkyl-OH. In a particular embodiment, the aryl group is optionally substituted with —O—C₁-C₆alkyl, or -Hal. In a particular embodiment, the aryl group is optionally substituted with —O—CH₃, or -Hal, wherein the halogen (-Hal) is, preferably, chloro.

More preferably, R³ is independently selected from the group consisting of 5- or 6-membered heterocycloalkyl containing one or two heteroatoms, wherein at least one heteroatom is O, C₅-C₆aryl, and 5- or 6-membered heteroaryl containing one or two heteroatoms. The heteroatom(s) in heterocycloalkyl and heteroaryl, respectively, is/are independently selected from N, O and S. At least one heteroatom is independently selected from O and S. The heterocycloalkyl, aryl or heteroaryl can be optionally substituted with —C₁-C₆alkyl, —O—C₁-C₆alkyl, -Hal, or —C₁-C₆alkyl-OH.

In one preferred embodiment, R³ is 5- or 6-membered heterocycloalkyl containing one heteroatom, wherein the heteroatom is O. The heterocycloalkyl is optionally substituted with —C₁-C₆alkyl, —O—C₁-C₆alkyl, -Hal, or —C₁-C₆alkyl-OH.

In another preferred embodiment, R³ is 5- or 6-membered heteroaryl containing one or two heteroatoms independently selected from N, O and S, wherein at least one heteroatom is independently selected from O and S. The heteroaryl is optionally substituted with one substituent selected from —C₁-C₆alkyl, —O—C₁-C₆alkyl, -Hal, or —C₁-C₆alkyl-OH.

In another preferred embodiment, R³ is 5- or 6-membered heteroaryl containing two heteroatoms, wherein one heteroatom is independently selected from O and S, and the second heteroatom is N. The heteroaryl is optionally substituted with one substituent selected from —C₁-C₆alkyl, —O—C₁-C₆alkyl, -Hal, or —C₁-C₆alkyl-OH.

In another embodiment, R³ is selected from the following:

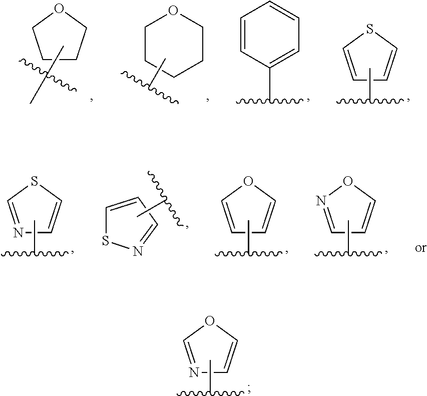

each of them can be optionally substituted with —C₁-C₆alkyl, —O—C₁-C₆alkyl, -Hal, or —C₁-C₆alkyl-OH.

More preferably, R³ is selected from the following:

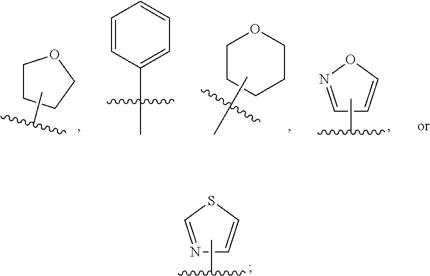

each of them can be optionally substituted with —C₁-C₆alkyl, —O—C₁-C₆alkyl, -Hal, or —C₁-C₆alkyl-OH.

In another embodiment, R³ is selected from the following:

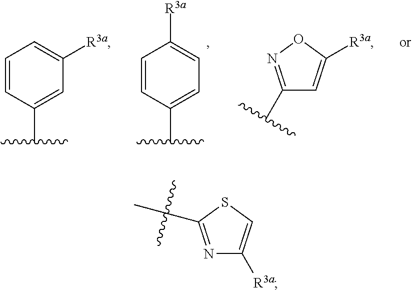

wherein R^{3a} is selected from hydrogen, halogen, —O—C₁-C₆alkyl, —C₁-C₆ alkyl-OH, or C₁-C₆alkyl.

Preferably, R^{3a} is selected from hydrogen, chloro, methoxy, methyl, isopropyl, or

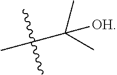 [SMILES: *C(C)(C)O]

In R³, C₁-C₆alkyl is preferably methyl, ethyl, isopropyl, propyl, isobutyl or butyl and can be optionally substituted with —OH such as

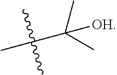 [SMILES: *C(C)(C)O]

In another embodiment, R² is hydrogen or C₁-C₆alkyl, and R³ is
a)

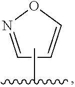

preferably

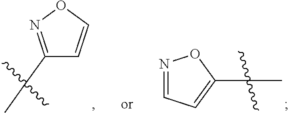 [SMILES: *c1ccon1 ; *c1ccno1]

b)

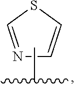

preferably

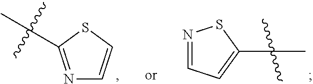 [SMILES: *c1nccs1 ; *c1ccns1]

c)

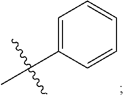 [SMILES: *c1ccccc1]

d)

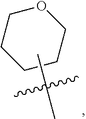

preferably

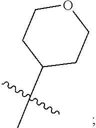 [SMILES: *C1CCOCC1]

e)

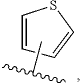

preferably

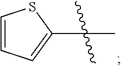 [SMILES: *c1cccs1 ; ";"]

f)

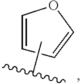

preferably

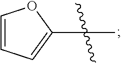 [SMILES: *c1ccco1 ; ";"]

g)

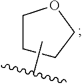

preferably

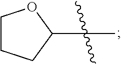 [SMILES: *C1CCCO1 ; ";"]

In another embodiment, R² is hydrogen or C₁-C₆alkyl, and R³ is
a)

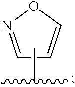

preferably

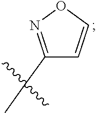 [SMILES: *c1ccon1]

b)

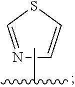

preferably

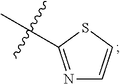 [SMILES: *c1nccs1]

c)

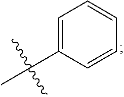 [SMILES: *c1ccccc1]

d)

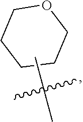

preferably

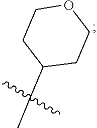 [SMILES: *C1CCOCC1]

or

In each of the above embodiments, R³ can be optionally substituted with halogen, —O—C₁-C₆alkyl, —C₁-C₆alkyl-OH, or C₁-C₆alkyl. More preferably, R³ can be optionally substituted with chloro, methoxy, methyl or isopropyl or

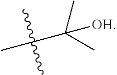 [SMILES: *C(C)(C)O]

In R³, C₁-C₆alkyl is preferably methyl, ethyl, isopropyl, propyl, isobutyl or butyl and can be optionally substituted with —OH such as in

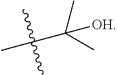 [SMILES: *C(C)(C)O]

Preferably, R² is C₁-C₆alkyl, wherein R² is preferably selected from the group consisting of methyl, ethyl, propyl, isopropyl, butyl, tert-butyl, pentyl and hexyl. More preferably, R² is C₁-C₃alkyl. More preferably, R² is independently selected from the group consisting of methyl, ethyl, propyl and isopropyl. Even more preferably, R² is independently selected from the group consisting of ethyl and isopropyl, most preferably ethyl.

In another preferred embodiment, R² is hydrogen.

In a further embodiment, the present invention relates to stereoisomers of a compound of formula (II) which are defined as compounds of formula (IIa) or (IIb)

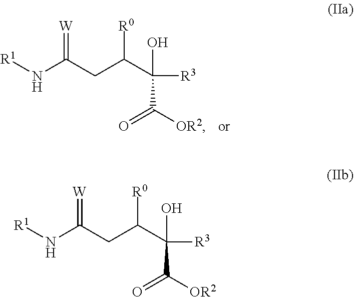

or a stereoisomer, a racemic mixture, a tautomer, a polymorph, a pharmaceutically acceptable salt, a prodrug, a hydrate, or a solvate thereof, wherein W, R⁰, R¹, R² and R³ are defined as above. Embodiments as defined for compounds of formula (II) apply here.

In a further embodiment, the present invention relates to the following compound of formula (II):

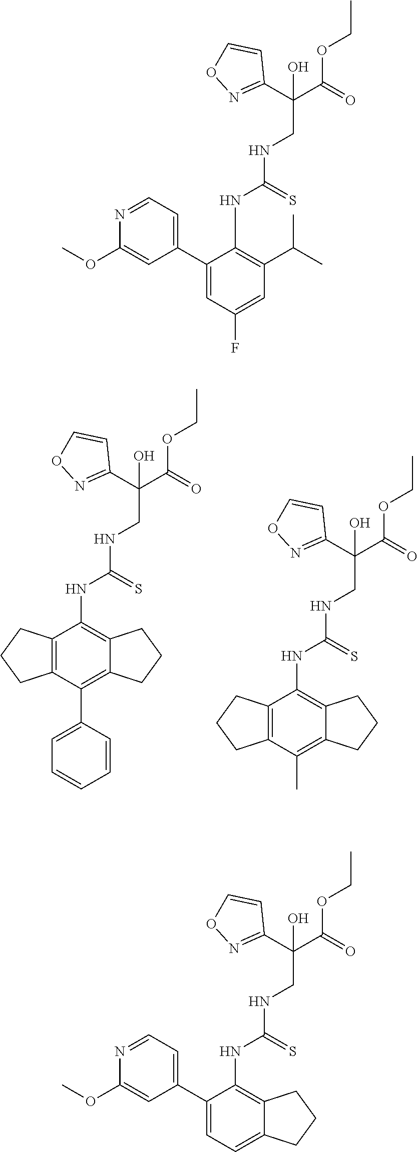

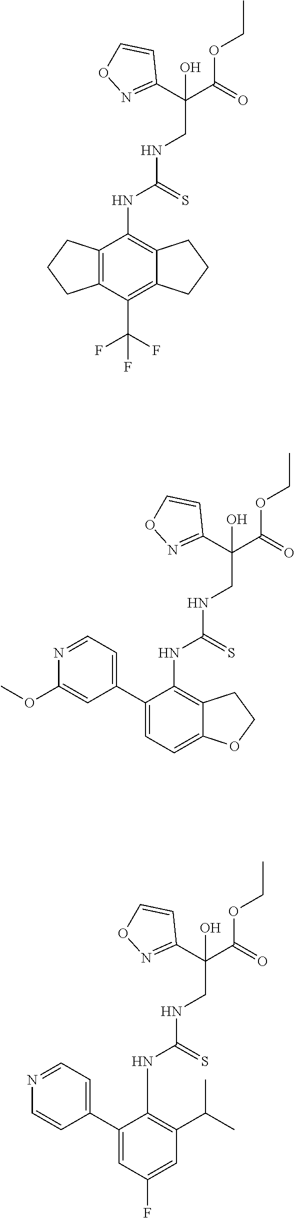

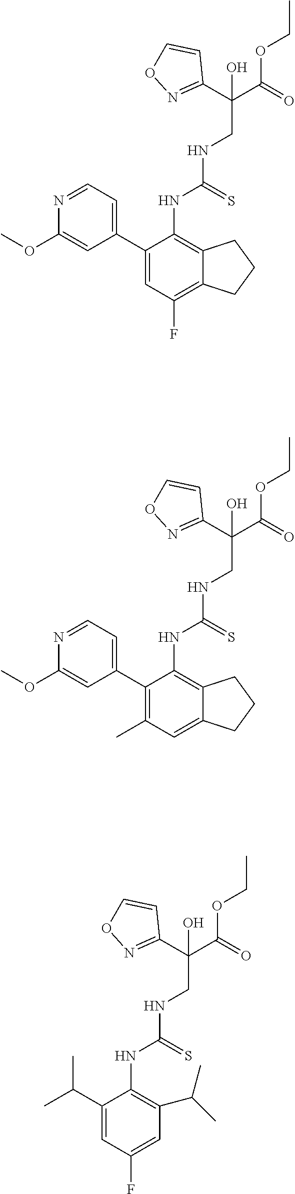

or stereoisomers, or racemic mixtures, or tautomers, or polymorph, or pharmaceutically acceptable salts, or hydrates, or solvates thereof.

The present invention relates further to a pharmaceutical composition comprising a compound of formula (I) as defined in the present invention, or a stereoisomer, a racemic mixture, a tautomer, a polymorph, a pharmaceutically acceptable salt, a prodrug, a hydrate, or a solvate thereof, and optionally at least one pharmaceutically acceptable carrier, diluent, adjuvant or excipient.

In one embodiment, the pharmaceutical composition comprises a compound of formula (I) as defined in the present invention, or a stereoisomer, a racemic mixture, a tautomer, a polymorph, a pharmaceutically acceptable salt, a prodrug, a hydrate, or a solvate thereof, and optionally at least one pharmaceutically acceptable carrier, diluent, adjuvant or excipient.

Various embodiments of the invention are described herein, it will be recognized that features specified in each embodiment may be combined with other specified features to provide further embodiments of the present invention.

The present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use as a medicament.

The present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels. In one embodiment, the modulation is the reduction and/or inhibition of IL-1 beta and/or IL-1 beta levels. Particularly, the modulation is the reduction and/or inhibition of IL-1 beta.

In another embodiment, the present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use in a method of reducing and/or inhibiting IL-1 beta. In particular, inhibiting IL-1 beta.

The present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of a component of the NLRP3 inflammasome pathway.

The present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of NLRP3 inflammasome pathway.

The present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular decrease, of IL-1 beta and/or IL-18 levels.

In other words, the present invention relates to a method for treating, alleviating or preventing of a disease, disorder or abnormality which is responsive to the modulation, in particular inhibition of activation, of a component of the NLRP3 inflammasome pathway and/or which is responsive to the modulation, in particular decrease, of the IL-1 beta and/or IL-18 levels, wherein the method comprises administering a therapeutically effective amount of a compound of formula (I) as defined in the present invention, or a stereoisomer, a racemic mixture, a tautomer, a polymorph, a pharmaceutically acceptable salt, a prodrug, a hydrate, or a solvate thereof, to a subject in need thereof (e.g. patient).

In one embodiment, the present invention relates to a method for treating, preventing or alleviating a disease, a disorder or abnormality which is responsive to the modulation, in particular inhibition of activation, of a component of the NLRP3 inflammasome pathway, wherein the method comprises administering a therapeutically effective amount of a compound of formula (I) as defined in the present invention, or a stereoisomer, a racemic mixture, a tautomer, a polymorph, a pharmaceutically acceptable salt, a prodrug, a hydrate, or a solvate thereof, to a subject in need thereof (e.g. a patient).

The present invention further relates to a method for treating, preventing or alleviating a disease, a disorder or abnormality which is responsive to the modulation, in particular inhibition of activation, of NLRP3 inflammasome pathway, wherein the method comprises administering a therapeutically effective amount of a compound of formula (I) as defined in the present invention, or a stereoisomer, a racemic mixture, a tautomer, a polymorph, a pharmaceutically acceptable salt, a prodrug, a hydrate, or a solvate thereof, to a subject in need thereof (e.g. a patient).

In one embodiment, the present invention relates to a method for treating, preventing or alleviating a disease, disorder or abnormality responsive to a modulation, in particular a decrease, of IL-1 beta and/or IL-18 levels, wherein the method comprises administering a therapeutically effective amount of a compound of formula (I) as defined in the present invention, or a stereoisomer, a racemic mixture, a tautomer, a polymorph, a pharmaceutically acceptable salt, a prodrug, a hydrate, or a solvate thereof, to a patient in need thereof.

The present invention relates to the use of a compound of formula (I) as defined in the present invention, or a stereoisomer, a racemic mixture, a tautomer, a polymorph, a pharmaceutically acceptable salt, a prodrug, a hydrate, or a solvate thereof, for the manufacture of a medicament. In a further embodiment, the present invention relates to the use of a compound of formula (I) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels. In one embodiment, the disease, disorder, or abnormality is selected from the list disclosed herein.

The present invention relates to the use of a compound of formula (I) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway.

The present invention relates to the use of a compound of formula (I) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of NLRP3 inflammasome pathway.

The present invention relates to the use of a compound of formula (I) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular decrease, of IL-1 beta and/or IL-18 levels.

The present invention relates to the use of a compound of formula (I) as defined in the present invention, or a stereoisomer, a racemic mixture, a tautomer, a polymorph, a pharmaceutically acceptable salt, a prodrug, a hydrate, or a solvate thereof, for the manufacture of a medicament for reducing and/or inhibiting IL-1 beta and/or IL-1 beta levels. In one embodiment, the present invention relates to the use of a compound of the invention, as defined herein, for the manufacture of a medicament for reducing and/or inhibiting IL-1 beta. In another embodiment, the present invention relates to the use of a compound of the invention, as defined herein, for the manufacture of a medicament for reducing IL-1 beta.

In one embodiment, the present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a tauopathy by modulating a component of the inflammasome pathway, in particular, by modulating the NLRP3 inflammasome pathway.

In another embodiment, the disease, the disorder or the abnormality is responsive to modulation of one or more of IL-1β, IL-17, IL-18, IL-1 a, IL-37, IL-33 and Th17 cells, preferably: IL-1β and IL-18.

In yet another embodiment, the disease, disorder, or abnormality is a disease, disorder, or abnormality selected from Alzheimer's disease, Parkinson's disease, cryopyrin-associated periodic syndromes (CAPS), non-alcoholic fatty liver disease (NAFLD), non-alcoholic steatohepatitis (NASH), and gout.

In another embodiment, the disease, disorder, or abnormality is a disease, disorder, or abnormality selected multiple sclerosis, hidradenitis suppurativa (HS) and chronic kidney disease.

In a further embodiment, the disease, disorder, or abnormality is a disease, a disorder or an abnormality of the immune system. In an embodiment, the disease, disorder, or abnormality is an inflammatory disease, disorder, or abnormality. In yet another embodiment, the disease, disorder, or abnormality is an autoimmune disease, disorder, or abnormality. In yet another embodiment, the disease, the disorder, or the abnormality is a disease, a disorder, or an abnormality of the central nervous system (CNS). In yet another embodiment, the disease, the disorder, or the abnormality can be a disease, disorder or abnormality or condition of the skin. The disease, the disorder or the abnormality can be a disease, disorder or abnormality or condition of the cardiovascular system. The disease, the disorder or the abnormality or condition can be a cancer, tumor or other malignancy. The disease, the disorder or the abnormality or condition can be a disease, disorder, or abnormality of the renal system. The disease, the disorder or the abnormality or condition can be a disease, disorder, or abnormality of the gastrointestinal tract. The disease, the disorder or the abnormality or condition can be a disease, disorder, or abnormality of the respiratory system. The disease, the disorder or the abnormality or condition can be a disease, disorder, or abnormality of the endocrine system. The disease, the disorder or the abnormality or condition can be liver related disease, disorder, or abnormality.

In one embodiment, the diseases, the disorders or the abnormalities which are responsive to the modulation, in particular inhibition of activation, of a component of the NLRP3 inflammasome pathway can be selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury.

In another embodiment, the diseases, the disorders or the abnormalities which are responsive to the modulation, in particular inhibition of activation, of a component of the NLRP3 inflammasome pathway can be selected from Edema (DME), Geographic Atrophy (GA), and Coronavirus-associated respiratory distress syndrome (CARDS).

Preferably, the diseases, the disorders or the abnormalities are selected from Alzheimer's disease, Parkinson's disease, amyotrophic lateral sclerosis, demyelination, viral encephalitis, epilepsy, stroke, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

In another preferred embodiment the diseases, the disorders or the abnormalities are selected from multiple sclerosis, traumatic brain injury, spinal cord injury, acute kidney disease, chronic kidney disease, acne, atopic dermatitis and hidradenitis suppurativa (HS).

More preferably, the diseases, the disorders or the abnormalities are selected from Alzheimer's disease, Parkinson's disease, amyotrophic lateral sclerosis, demyelination, viral encephalitis, epilepsy, stroke, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 and type 2 diabetes, rheumatoid arthritis, and myelodysplastic syndrome.

Even more preferably, the diseases, the disorders or the abnormalities are selected from Alzheimer's disease, Parkinson's disease, cryopyrin-associated periodic syndromes (CAPS), non-alcoholic fatty liver disease (NAFLD), non-alcoholic steatohepatitis (NASH), rheumatoid arthritis and gout. Even more preferably, the diseases, the disorders or the abnormalities are selected from Alzheimer's disease, Parkinson's disease, cryopyrin-associated periodic syndromes (CAPS), rheumatoid arthritis and gout.

In one embodiment, the present invention relates to a compound of formula (I) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a IL-18 and/or IL-1 beta related disease by modulating a component of the NLRP3 inflammasome pathway, in particular, by modulating NLRP3 inflammasome pathway. The IL-18 and/or IL-1 beta levels in a subject are decreased as a result of the administration of a compound of formula (I) as defined in the present invention, or a stereoisomer, a racemic mixture, a tautomer, a polymorph, a pharmaceutically acceptable salt, a prodrug, a hydrate, or a solvate thereof.

IL-18 and/or IL-1 beta related diseases, disorders or abnormalities are selected from chronic obstructive pulmonary disease (COPD), transfusion-related lung injury, bronchopulmonary dysplasia (BPD), acute respiratory distress syndrome (ARDS), pediatric autoinflammatory disease or condition, Still's disease, particularly Adult Still's disease or juvenile Still's disease, juvenile rheumatoid arthritis (JRA), juvenile idiopathic arthritis (JIA), systemic juvenile onset idiopathic arthritis (SoJIA), systemic juvenile idiopathic arthritis (sJIA), interstitial lung disease (ILD), macrophage activation syndrome (MAS) including primary, secondary and recurrent MAS, hemophagocytic lymphohistiocytosis (HLH), Familial (hereditary) hemophagocytic lymphohistiocytosis (FHLH) associated with gene defects in perforin, munc 13-4 and 18-2, synthaxin 11, immune deficiencies such as Chediak-Higashi syndrome (CHS), Griscelli syndrome (GS), X-linked lymphoproliferative syndrome (XLP2), X-linked inhibitor of apoptosis protein deficiency (XIAP), acquired hemophagocytic lymphohistiocytosis associated with infectious conditions especially Herpes virus such as EBV and other pathogens, autoinflammatory syndrome associated with NLRC4 mutations, Giant Cell Arteritis (GCA), acne, pyogenic arthritis pyoderma gangrenosum and acne (PAPA), pulmonary sarcoidosis, heart failure, ischemic heart disease, dry eye disease (DED), keratitis, corneal ulcer and abrasion, iritis, glaucoma, Sjogren's syndrome, autoimmune uveitis, Behcet's disease, conjunctivitis, allergic conjunctivitis, diabetes type 2, solid organ and hematologic stem cell transplantation, ischemia reperfusion injury, familial Mediterranean fever (FMF), tumor necrosis factor receptor 1-associated periodic syndromes (TRAPS), hyper-IgD syndromes (mevalonate kinase gene mutation), gout, Schnitzler syndrome, Wegener's granulomatosis also called granulomatosis with polyangitis (GPA), Hashimoto's thyroiditis, Crohn's disease, early onset inflammatory bowel disease (EOIBD), very EOIBD (VEOIBD), infantile IBD, neonatal IBD, ulcerative colitis and Blau syndrome (NOD-2 mutation).

The modulation of NLRP3 inflammasome pathway appears to be beneficial in diseases or disorders or abnormalities with altered IL-18 levels and/or IL-1 beta, which lead to pathological inflammation.

The present invention relates to compound of formula (I) as defined in the present invention that are modulators of NLRP3 inflammasome activity and/or modulators of IL-18 and/or IL-1b levels in a subject.

In one embodiment, the invention provides a pharmaceutical composition comprising a compound of formula (I) as defined in the present invention, or a stereoisomer, a racemic mixture, a tautomer, a polymorph, a pharmaceutically acceptable salt, a prodrug, a hydrate, or a solvate thereof, and at least one further biologically active compound. Optionally, the pharmaceutical combination may comprise a pharmaceutically acceptable carrier, diluent, adjuvant or excipient as described herein.

In another embodiment, the present invention relates to a pharmaceutical composition comprising a combination of a compound of formula (I) as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I) and optionally comprising at least one pharmaceutically acceptable carrier, diluent, adjuvant or excipient.

In another embodiment, the present invention relates to a pharmaceutical composition comprising a combination of a compound of formula (I) as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I), and optionally comprising at least one pharmaceutically acceptable carrier, diluent, adjuvant or excipient.

In particular the further biologically active compound can be one used for the treatment of a disease, disorder or abnormality which targets a different pathomechanism, e.g. an anti-amyloid beta antibody, anti-Tau antibody, amyloid beta small molecule inhibitor, Tau aggregation small molecule inhibitor, anti-alpha synuclein antibody or alpha-synuclein aggregation small molecule inhibitor, anti-TDP-43 antibody or anti-TDP-43 aggregation small molecule inhibitor, among others. When a compound of the invention is used in combination with a further biologically active compound, the dose of each compound may differ from the dose if the compound is used as a monotherapy. Such biologically active compounds are well known from the literature. Such biological active compound is, for example, a chemical compound, peptide, antibody, antibody fragment, or nucleic acid, which is therapeutically active or enhances the therapeutic activity when administered to a subject (e.g., patient) in combination with a compound of the invention.

In another embodiment, the present invention relates to a pharmaceutical composition comprising a combination comprising a compound of formula (I) as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I), and optionally comprising at least one pharmaceutically acceptable carrier, diluent, adjuvant or excipient, for use as a medicament.

The term “combination” refers to either a fixed combination in one dosage unit form, or a combined administration where a compound of the present invention and a combination partner (e.g. another drug as explained above, also referred to as “therapeutic agent” or “further biologically active compound”) may be administered independently at the same time or separately within time intervals.

In another embodiment, the present invention relates to combination, in particular a pharmaceutical combination, comprising a therapeutically effective amount of a compound of formula (I) as defined in the present invention, or a stereoisomer, a racemic mixture, a tautomer, a polymorph, a pharmaceutically acceptable salt, a prodrug, a hydrate, or a solvate thereof, and at least one further biologically active compound, and optionally at least one pharmaceutically acceptable carrier, diluent, adjuvant or excipient. In particular, the at least one further biologically active compound is a compound differing from a compound of formula (I).

In another embodiment, the present invention relates to a combination comprising a therapeutically effective amount of a compound of formula (I) as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I), and optionally comprising at least one pharmaceutically acceptable carrier, diluent, adjuvant or excipient, for use as a medicament.

The present invention relates to the use of a compound of formula (I) as defined in the present invention, or a stereoisomer, a racemic mixture, a tautomer, a polymorph, a pharmaceutically acceptable salt, a prodrug, a hydrate, or a solvate thereof, as an analytical reference or an in vitro screening tool. The compounds of the present invention, or a stereoisomer, a racemic mixture, a tautomer, a polymorph, a pharmaceutically acceptable salt, a prodrug, a hydrate, or a solvate thereof, can be used as an analytical reference or an in vitro screening tool for characterization of cells with activated NLRP3 inflammasome pathway and for testing of compounds targeting the NLRP3 inflammasome pathway.

Accordingly, the invention provides the use of a compound of formula (I) as defined in the present invention, or a stereoisomer, a racemic mixture, a tautomer, a polymorph, a pharmaceutically acceptable salt, a prodrug, a hydrate, or a solvate thereof, for treating, alleviating or preventing a disorder or an abnormality which is responsive to the modulation of a component of the NLRP3 inflammasome pathway or which is responsive to the modulation, in particular decrease, of IL-1 beta and/or IL-18 levels, wherein the medicament is prepared for administration with further biologically active agent. The invention also provides the use of further biologically active agent for treating alleviating or preventing a disorder or an abnormality which is responsive to the modulation of a component of the NLRP3 inflammasome pathway or which is responsive to the modulation, in particular decrease, of IL-1 beta and/or IL-18 levels, wherein the further biologically active agent is administered with a compound of the present invention, or a stereoisomer, a racemic mixture, a tautomer, a polymorph, a pharmaceutically acceptable salt, a prodrug, a hydrate, or a solvate thereof.

In another embodiment, the invention provides the use of a compound of formula (I) as defined in the present invention, or a stereoisomer, a racemic mixture, a tautomer, a polymorph, a pharmaceutically acceptable salt, a prodrug, a hydrate, or a solvate thereof, for treating, alleviating or preventing a disorder or an abnormality which is responsive to the modulation of a component of the NLRP3 inflammasome pathway or which is responsive to the modulation, in particular decrease, of IL-1 beta and/or IL-18 levels, wherein the modulation is the reduction and/or the inhibition of IL-1 beta and/or IL-1 beta levels. Preferably, the modulation is the reduction and/or the inhibition of IL-1 beta. Preferably, the modulation is the inhibition of IL-1 beta. In another embodiment, the invention provides a compound of formula (I) as defined in the present invention, or a stereoisomer, a racemic mixture, a tautomer, a polymorph, a pharmaceutically acceptable salt, a prodrug, a hydrate, or a solvate thereof, for use as a medicament, in particular for inhibiting IL-1 beta.

In another embodiment, the invention also provides a compound of formula (I) as defined in the present invention, or a stereoisomer, a racemic mixture, a tautomer, a polymorph, a pharmaceutically acceptable salt, a prodrug, a hydrate, or a solvate thereof, for use in a method of treating, alleviating or preventing a disease, disorder or abnormality which is responsive to the modulation of a component of the NLRP3 inflammasome pathway or which is responsive to the modulation, in particular decrease, of IL-1 beta and/or IL-18 levels, wherein said compound of formula (I) is prepared for administration with further biologically active compound (as defined herein).

In another embodiment, the present invention also provides a method of treating alleviating or preventing a disease, disorder or abnormality which is responsive to the modulation of a component of the NLRP3 inflammasome pathway or which is responsive to the modulation, in particular decrease, of IL-1 beta and/or IL-18 levels, selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury; preferably the disorder is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs), comprising administering to the subject a therapeutically effective amount of a compound of formula (I), as defined herein, or stereoisomers, or racemic mixtures, or tautomers, or polymorph, or pharmaceutically acceptable salts, or hydrates, or solvates thereof.

In another embodiment, the disease, disorder or abnormality can be selected from Edema (DME), Geographic Atrophy (GA), Coronavirus-associated respiratory distress syndrome (CARDS), multiple sclerosis, traumatic brain injury, spinal cord injury, acute kidney disease, chronic kidney disease, acne, atopic dermatitis and hidradenitis suppurativa (HS).

In another embodiment, the present invention also provides a method of inhibiting IL-1 beta in a subject in need, the method comprising administering to a subject in need thereof a therapeutically effective amount of a compound of formula (I), or a stereoisomer, a racemic mixture, a tautomer, a polymorph, a pharmaceutically acceptable salt, a prodrug, a hydrate, or a solvate thereof.

In particular, the disease, disorder or abnormality is one which is responsive to the inhibition of activation of the NLRP3 inflammasome pathway. More particularly, the disease, disorder or abnormality is responsive to the modulation of one or more of, for example, but not limited to, IL-1 β or IL-18. For example, the disease, disorder, or abnormality is responsive to the modulation of one or more of IL-1 β, IL-17, IL-18, IL-1 a, IL-37, IL-33 and Th17 cells, preferably the disease, disorder, or abnormality is responsive to the modulation of IL-1 β and/or IL-18.

### Method of Synthesizing the Compounds of the Present Invention

The compounds of the present invention can be prepared in accordance with the definition of a compound of formula (I), as disclosed herein, by the synthesis routes described in the following schemes or examples. All methods described herein can be performed in any suitable order unless otherwise indicated herein or otherwise clearly contradicted by the context. The use of any and all examples, or exemplary language (e.g. such as, preferably) provided herein is intended merely to better illustrate the invention and does not pose a limitation on the scope of the invention otherwise claimed.

In the following general methods, R⁰, R¹, R², R³, X, Y, and W are as previously defined in the above embodiments or limited to the designation in the schemes. Unless otherwise stated, starting materials are either commercially available or prepared via know methods.

The present invention relates to a method for preparing a compound of formula (I), as disclosed herein.

In one embodiment, the method comprises the step of cyclization of a compound of formula (II) for preparing a compound of formula (I) in the presence of a condensation agent:

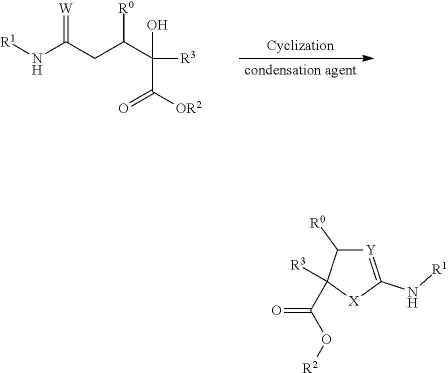

wherein W, X, Y, R¹, R², R³ and R⁰ are as defined above.

Preferably, the present method comprises the step of cyclization of a compound of formula (II) for preparing a compound of formula (I) in the presence of a condensation agent.

The condensation agent is used for cyclization of a compound of formula (II) for preparing a compound of formula (I) in the presence of a base. The base is preferably trimethylamine (Et₃N). The condensation agent is preferably N-(3-dimethylaminopropyl)-N′-ethylcarbodiimide (EDC), Mukaiyama reagent, iodomethane or TsCI. The cyclization is conducted preferably at room temperature (rt).

Any combination of the embodiments, preferred embodiments and more preferred embodiments disclosed herein is also envisaged in the present invention.

### Pharmaceutical Compositions

While it is possible for the compounds of the present invention, or a stereoisomer, a racemic mixture, a tautomer, a polymorph, a pharmaceutically acceptable salt, a prodrug, a hydrate, or a solvate thereof, to be administered alone, it is preferable to formulate them into a pharmaceutical composition in accordance with standard pharmaceutical practice. Thus, the invention also provides a pharmaceutical composition which comprises a therapeutically effective amount of a compound of formula (I) or a stereoisomer, a racemic mixture, a tautomer, a polymorph, a pharmaceutically acceptable salt, a prodrug, a hydrate, or a solvate thereof, optionally in admixture with a pharmaceutically acceptable carrier, diluent, adjuvant or excipient.

The term “a therapeutically effective amount” of a compound of the present invention refers to an amount of the compound of the present invention (i.e. a compound of formula (I), or a stereoisomer, a racemic mixture, a tautomer, a polymorph, a pharmaceutically acceptable salt, a prodrug, a hydrate, or a solvate thereof) that will elicit the biological or medical response of a subject, for example, reduction or inhibition of an enzyme or a protein activity, or ameliorate symptoms, alleviate conditions, slow or delay disease progression, or prevent a disease, a disorder or an abnormality, etc. In one embodiment, the term “a therapeutically effective amount” refers to the amount of the compound of the present invention that, when administered to a subject in need thereof (e.g. a patient), is effective to at least partially alleviate, prevent and/or ameliorate a disease, a disorder, or an abnormality which is responsive to the modulation of a component of the NLRP3 inflammasome pathway or which is responsive to the modulation, in particular decrease, of IL-1 beta and/or IL-18.

Pharmaceutically acceptable carriers, diluents, adjuvants and excipients are well known in the pharmaceutical art and are described, for example, in Remington's Pharmaceutical Sciences, 18^{th} Ed. (Alfonso R. Gennaro, ed.; Mack Publishing Company, Easton, PA, 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc, 1999); Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); Fiedler's “Lexikon der Hilfsstoffe” 5^{th} Ed., Edition Cantor Verlag Aulendorf 2002; “The Handbook of Pharmaceutical Excipients”, 4^{th} Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The carriers, diluents, adjuvants and pharmaceutical excipients can be selected with regard to the intended route of administration and standard pharmaceutical practice. These compounds must be acceptable in the sense of being not deleterious to the recipient thereof.

Pharmaceutically useful excipients that may be used in the formulation of the pharmaceutical composition of the present invention may comprise, for example, vehicles, solvents (such as monohydric alcohols such as ethanol, isopropanol and polyhydric alcohols such as glycols), edible oils (such as soybean oil, coconut oil, olive oil, safflower oil, and cottonseed oil), oily esters (such as ethyl oleate and isopropyl myristate), binders (such as hydroxypropylmethyl cellulose (HPMC), hydroxypropyl cellulose (HPC), pregelatinized starch and combinations thereof), solubilizers, thickening agents, stabilizers, disintegrants (such as carboxymethylcellulose calcium (CMC-Ca), carboxymethylcellulose sodium (CMC-Na), crosslinked PVP (e.g., crospovidone, Polyplasdone® or Kollidon® XL), alginic acid, sodium alginate, guar gum, cross-linked CMC (croscarmellose sodium, e.g. Ac-Di-Sol®), carboxymethyl starch-Na (sodium starch glycolate) (e.g., Primojel® or Explotab®), preferably crosslinked PVP and/or croscarmellose sodium), glidants (such as colloidal SiO₂ (e.g., Aerosil® 200), magnesium trisilicate, powdered cellulose, talc and combinations thereof), lubricating agents (such as magnesium stearate, aluminium or calcium silicate, stearic acid, hydrogenated castor oil, talc, glyceryl behenate, sodium stearate fumarate and combinations thereof), buffering agents, emulsifiers, wetting agents, suspending agents, sweetening agents, colorants, flavors, coating agents, preservatives, antioxidants, processing agents, drug delivery modifiers and enhancers (such as calcium phosphate), magnesium stearate, talc, monosaccharides, disaccharides, starch, gelatine, cellulose, methylcellulose, sodium carboxymethyl cellulose, dextrose, hydroxypropyl-β-cyclodextrin, polyvinylpyrrolidone, low melting waxes, and ion exchange resins.

The carrier is not particularly limited and will depend on the route of administration as well as the form of the pharmaceutical composition (i.e., solid, liquid, etc.). Suitable carriers include, without limitation, polyols such as mannitol, sorbitol, xylitol; disaccharides such as lactose, sucrose, dextrose and maltose; polysaccharides such as maltodextrine and dextranes; starches such as corn starch; celluloses such as microcrystalline cellulose, sodium carboxy methylcellulose, low-substituted hydroxypropyl cellulose, hydroxyl ethyl cellulose, hydroxypropyl cellulose or mixtures thereof; cylodextrines and inorganic agents such as dicalcium phosphate, calcium hydrogen phosphate; hydroxyapatite, tricalcium phosphate, talcum and silica. Microcrystalline cellulose, sucrose and/or lactose are preferred as carriers. Combinations thereof can also be employed. Carriers can include also protein and cell penetrating peptides which should be selected depending on the route of administration and target.

The diluent is not particularly limited and will depend on the route of administration as well as the form of the pharmaceutical composition (i.e., solid, liquid, etc.). Diluents include, for instance, water, ethanol, propylene glycol and glycerin, and combinations thereof.

An adjuvant is an additive which has few or no pharmacological effects by themselves, but that increases the efficacy or potency of the compounds of the invention if they are administered together.

The routes for administration (delivery) of the compounds of the invention include, but are not limited to, one or more of the following routes of administration: oral (e.g., as a tablet, capsule, or as an ingestible solution), topical, mucosal (e. g. as a nasal spray or aerosol for inhalation), nasal, parenteral (e.g., by an injectable form), gastrointestinal, intraspinal, intraperitoneal, intramuscular, intravenous, intraarterial, intrathecal, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

For example, the compounds can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatine and acacia. Additionally, lubricating agents such as magnesium stearate, stearic acid, glyceryl behenate and talc may be included. Solid compositions of a similar type may also be employed as fillers in gelatine capsules. Preferred excipients in this regard include starch, cellulose, milk sugar e.g. lactose or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

If the compounds of the present invention, as disclosed herein, are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds can be used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compounds of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e. g. using a mixture of ethanol and the propellant as the solvent, which may additionally contain a lubricant, e. g. sorbitan trioleate. Capsules and cartridges (made, for example, from gelatine) for use in an inhaler or insufflator may be formulated to contain a powder mix of the compound and a suitable powder base such as lactose or starch.

Alternatively, the compounds of the present invention, as defined herein, can be administered in the form of a suppository or pessary, or it may be applied topically in the form of a gel, hydrogel, lotion, solution, cream, ointment or dusting powder. The compounds of the present invention, as defined herein, may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, polyethylene glycol, liquid paraffin, polysorbate 60, cetyl ester wax, cetearyl alcohol, 2-octyldodecanol, benzyl alcohol and water.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The claimed compounds, as defined herein, can be used for the treatment, alleviation or prevention of the recited conditions alone or in combination with one or more other biologically active compounds, as defined herein. In particular, the other biologically active compound can be one used for the treatment, alleviation, or prevention of the recited diseases.

The combinations referred to above may conveniently be presented for use in the form of a pharmaceutical formulation. The individual components of such combinations may be administered either sequentially or simultaneously in separate or combined pharmaceutical formulations by any convenient route. When administration is sequential, either the compound of the invention or the other biologically active compound may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manners as are known for such compounds in the art.

The pharmaceutical compositions of the invention can be produced in a manner known per se to the skilled person as described, for example, in Remington's Pharmaceutical Sciences, 15^{th} Ed., Mack Publishing Co., New Jersey (1975).

The compounds according to the present invention, as disclosed herein, can also be provided in the form of a mixture with at least one further biologically active compound and/or a pharmaceutically acceptable carrier, diluent, adjuvant, or excipient. The compound and/or the further biologically active compound are preferably present in a therapeutically effective amount.

The nature of the further biologically active compound will depend on the intended use of the mixture. The further biologically active substance or compound may exert its biological effect by the same or a similar mechanism as the compound according to the invention or by an unrelated mechanism of action or by a multiplicity of related and/or unrelated mechanisms of action.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and delectability. ¹⁸F-labeled compounds are particularly suitable for imaging applications such as PET. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

### METHOD OF USE OF THE INVENTION

There is evidence for a role of NLRP3-induced IL-1 and IL-18 in the inflammatory responses occurring in connection with, or as a result of, a multitude of different diseases, disorders or abnormalities which is responsive to the modulation of a component of the NLRP3 inflammasome pathway and/or which is responsive to the modulation of IL-1 beta and/or IL-18 levels. (Menu et al., Clinical and Experimental Immunology, 2011, 166, 1-15; Strowig et al., Nature, 2012, 481, 278-286).

The invention provides a compound of formula (I), as defined herein, or a stereoisomer, a racemic mixture, a tautomer, a polymorph, a pharmaceutically acceptable salt, a prodrug, a hydrate, or a solvate thereof, which exhibit valuable pharmacological properties, e.g. NRLP3 inhibiting properties on the NLRP3 inflammasome pathway. Said compounds of the invention may be useful in the treatment, alleviation or prevention of a disease, or a disorder or an abnormality which is responsive to the modulation of a component of the NLRP3 inflammasome pathway and/or which is responsive to the modulation of IL-1 beta and/or IL-18 levels. A number of diseases, disorders or abnormalities have been shown to be involve in NLRP3 including, for example, one of the following:
A. Central nervous system disease (CNS), disorder, or abnormality, such as Alzheimer's disease, Parkinson's disease, dementia, frontotemporal dementia, Huntington's disease, cerebral malaria, brain injury from pneumococcal meningitis, motor neuron disease, traumatic brain injury, spinal cord injury, neuropathic pain, amyotrophic lateral sclerosis, migraine or multiple sclerosis (MS);
B. Immune disease, disorder, or abnormality (e.g. autoimmune disease, disorder or abnormality, and disease, disorder, or abnormality, involving the immune system), such as type 1 diabetes, hidradenitis suppurativa (HS), Schnitzler syndrome, multiple sclerosis (MS) including primary progressive multiple sclerosis (PPMS), Sjogren's syndrome, secondary progressive multiple sclerosis (SPMS), TNF receptor associated periodic syndrome (TRAPS), graft-versus host disease, antiphospholipid syndrome, refractory celiac disease, autoimmune pancreatitis, or relapsing remitting multiple sclerosis (RRMS);
C. Inflammatory disease, including auto-inflammation and inflammation occurring as a result of an inflammatory disease, disorder, or abnormality, such as mevalonate kinase deficiency (MKD), hyperimmunoglobulinemia D, cryopyrin-associated periodic syndromes (CAPS), Muckle-Wells syndrome (MWS), familial cold autoinflammatory syndrome (FCAS), neonatal-onset multisystem inflammatory disease (NOMID), familial Mediterranean fever (FMF), acne, pyogenic arthritis, pyoderma gangrenosum and acne (PAPA), adult-onset Still's disease (AOSD), Majeed syndrome, PLCG2-associated antibody deficiency and immune dysregulation (PLAID), PLCG2-associated autoinflammation, antibody deficiency and immune dysregulation (APLAID), pyogenic arthritis, haploinsufficiency of A20 (HA20), pediatric granulomatous arthritis (PGA), or sideroblastic anemia with B-cell immunodeficiency, periodic fevers, pancreatitis, developmental delay (SIFD);
D. Skin disease, disorder, or abnormality including skin inflammatory conditions, such as hidradenitis suppurativa (HS), dermatitis, psoriasis, skin contact hypersensitivity, acne, periodic fever syndrome (HIDS), Sweet's syndrome, eczema, skin lesions, burn, wound, wound healing, trauma, sunburn, actinic keratosis, deficiency of interleukin 1 receptor (DIRA) antagonist, epidermolysis bullosa, vitiligo, atopic dermatitis, cutaneous lupus, or alopecia areata;
E. Ocular disease, disorder, or abnormality, such as age-related macular degeneration (AMD), corneal infection, uveitis, glaucoma, dry eye, Geographic Atrophy (GA), or demyelination;
F. Cardiovascular disease, disorder, or abnormality (e.g. disease, disorder, or abnormality of the cardiovascular system) such as myocardial infarction, hypertension, ischaemia reperfusion injury, pericarditis including Dressler's syndrome, aneurysms including abdominal aortic aneurism, heart failure, coronary artery disease, or stroke;
G. Metabolic disease, disorder, or abnormality, such as type 2 diabetes, Edema (DME), obesity, atherosclerosis, diabetes, gout, or pseudo-gout;
H. Respiratory disease, disorder, or abnormality (e.g. disease, disorder or abnormality of the respiratory system), such as asbestosis, silicosis, cystic fibrosis, allergic inflammation, chronic obstructive pulmonary disorder (COPD), Coronavirus-associated respiratory distress syndrome (CARDS), steroid-resistant asthma, or asthma;
I. Liver disease, disorder, or abnormality, (e.g. hepatic disease, disorder or abnormality) such as hepatitis, primary biliary cholangitis, alcoholic liver disease, alcoholic fatty liver disease (AFLD), alcoholic steatohepatitis (ASH), non-alcoholic fatty liver disease (NAFLD), cytokine release syndrome or non-alcoholic steatohepatitis (NASH) including advanced fibrosis stages F3 and F4;
J. Renal disease, disorder, or abnormality (e.g. disease, disorder or abnormality of the renal system) such as oxalate-induced nephropathy, diabetic nephropathy, chronic kidney disease, lupus nephritis or kidney disease;
K. Cancer disease, disorder, or abnormality (e.g. cancer, tumor, or malignancy), such as lung cancer (e.g. lung cancer metastasis), pancreatic cancers, gastric cancers, leukemia, myelodysplastic syndrome (MOS), skin cancer, tumors of the endocrine system, or thyroid cancer;
L. Infections including viral infections, such as helminth infections (e.g. from schistosoma, roundworms, tapeworms or flukes), viral encephalitis, bacterial infection, periodontitis, human immunodeficiency virus (HIV), HIV-associated neurocognitive disorder, chronic nonbacterial osteomyelitis (CNO), chronic bacterial osteomyelitis, deficiency of interleukin 1 receptor (DIRA) antagonist, or epilepsy; alphavirus (e.g. Chikungunya virus and Ross River virus), flaviviruses (e.g. Dengue and Zika virus), Coronavirus-associated inflammatory pathologies, Coronaviruses, or influenza virus;
M. Psychological disease, disorder, or abnormality, such as depression, and psychological stress;
N. Inflammation, including inflammation occurring as a result of an inflammatory disease, disorder, or abnormality, such as an autoinflammatory disease, inflammation occurring as a symptom of a non-inflammatory disorder, inflammation occurring as a result of infection, or inflammation secondary to trauma, injury or autoimmunity. Examples of inflammation include inflammatory responses occurring in connection with, or as a result of:
   i. A joint disease, disorder, or abnormality, such as periodic fever syndrome (HIDS), rheumatoid arthritis, pustulosis, synovitis, osteoarthritis, chronic recurrent multifocal osteomyelitis (CRMO), systemic juvenile idiopathic arthritis, osteitis syndrome (SAPHO), hyperostosis, relapsing polychondritis, ankylosing spondylitis, or adult-onset Still's disease;
   ii. A gastrointestinal disease, disorder, or abnormality (e.g. disease, disorder or abnormality of the gastrointestinal tract) such as colitis, ulcerative colitis, Chron's disease, or inflammatory bowel disease;
   iii. A muscular disease, disorder, or abnormality, such as polymyositis, or myasthenia gravis;
   iv. A disease, disorder or abnormality of the endocrine system, such as, diabetes, parathyroid disease (e.g. hypothyroidism), tumors of the endocrine system, thyroid cancer, or hypoglycemia; and/or
   v. A vascular disease, disorder or abnormality, such as Behcet's disease or mucocutaneous lymph node syndrome.

In one embodiment, the disease, disorder, or abnormality is selected from Alzheimer's disease, Parkinson's disease, cryopyrin-associated periodic syndromes (CAPS), non-alcoholic fatty liver disease (NAFLD), non-alcoholic steatohepatitis (NASH), and gout.

In particular, the disease, disorder or abnormality is selected from: Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome mevalonate (TRAPS), kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury; preferably the disorder is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

In yet another embodiment, the disease, disorder or abnormality is preferably an inflammatory disease, disorder or abnormality; or an autoimmune disease, disorder or abnormality; or a disease, disorder or abnormality of the skin (such as, for example, but not limited to, psoriasis, acne, eczema, alopecia areata, or actinic keratosis); or a disease, disorder or abnormality of the cardiovascular system; or a disease, disorder, or abnormality such as a cancer, a tumor or a malignancy; or a disease, disorder or abnormality of the renal system; a disease, disorder or abnormality of the gastrointestinal tract; a disease, disorder or abnormality of the respiratory system; or a disease, disorder or abnormality of the endocrine system; or a disease, disorder or abnormality of the central nervous system (CNS); or a disease, disorder or abnormality of the liver.

In one embodiment, the disease, disorder, or abnormality is selected from Edema (DME), Geographic Atrophy (GA), Coronavirus-associated respiratory distress syndrome (CARDS), multiple sclerosis, traumatic brain injury, spinal cord injury, acute kidney disease, chronic kidney disease, acne, atopic dermatitis and hidradenitis suppurativa (HS).

In one embodiment, the disease, the disorder or the abnormality is selected from Alzheimer's disease, Parkinson's disease, multiple sclerosis, cryopyrin-associated periodic syndromes (CAPS), non-alcoholic fatty liver disease (NAFLD), non-alcoholic steatohepatitis (NASH), hidradenitis suppurativa (HS), chronic kidney disease and gout.

In one embodiment, the disease, the disorder or the abnormality is selected from Alzheimer's disease, Parkinson's disease, cryopyrin-associated periodic syndromes (CAPS), non-alcoholic fatty liver disease (NAFLD), non-alcoholic steatohepatitis (NASH) and gout.

In another embodiment, the disease, disorder, or abnormality is selected from Alzheimer's disease, Parkinson's disease, cryopyrin-associated periodic syndromes (CAPS), non-alcoholic fatty liver disease (NAFLD), non-alcoholic steatohepatitis (NASH), and hidradenitis suppurativa (HS).

In particular, the disease, disorder or abnormality is selected from: Alzheimer's disease, Parkinson's disease, amyotrophic lateral sclerosis, demyelination, viral encephalitis, epilepsy, stroke, atherosclerosis, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease (IBD) (including Crohn's disease, ulcerative colitis), hepatitis, nonalcoholic fatty liver disease, non-alcoholic steatohepatitis, hypertension, myocardial infarction, heart failure, coronary artery disease, oxalate-induced nephropathy, graft-versus host disease, type 1 diabetes, type 2 diabetes, Edema (DME), Geographic Atrophy (GA), rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, pyogenic arthritis pyoderma gangrenosum and acne (PAPA), haploinsufficiency of A20 (HA20), PLCG2-associated antibody deficiency and immune granulomatous arthritis (PGA), PLCG2-associated dysregulation (PLAID), pediatric autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, skin contact hypersensitivity, sunburn, psoriasis, hidradenitis suppurativa (HS), epidermolysis bullosa, acne, eczema, alopecia areata, actinic keratosis, hyperostosis, osteitis syndrome (SAPHO), vitiligo, atopic dermatitis, cutaneous lupus, multiple sclerosis (MS), Behcet's disease, Sjogren's syndrome, Schnitzler syndrome, chronic obstructive pulmonary disorder (COPD), asthma, steroid-resistant asthma, Coronavirus-associated inflammatory pathologies including Coronavirus-associated respiratory distress syndrome (CARDS), asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, acute kidney injury, chronic kidney disease, lupus nephritis diabetic nephropathy, alcoholic liver disease, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, primary biliary cholangitis, antiphospholipid syndrome, refractory celiac disease, pancreatitis, autoimmune pancreatitis, mucocutaneous lymph node syndrome, lung cancer metastasis, pancreatic cancers, gastric cancers, myelodisplastic syndrome, leukemia; polymyositis, colitis, helminth infection, bacterial infection, abdominal aortic aneurism, wound healing, migraine, depression, psychological stress, pain, neuropathic pain, periodontitis, pericarditis including Dressler's syndrome, ischaemia reperfusion injury, frontotemporal dementia, HIV-associated neurocognitive disorder, traumatic brain injury, traumatic spinal cord injury, ankylosing spondylitis, cytokine release syndrome. Preferably, the diseases, the disorders or the abnormalities are selected from Alzheimer's disease, Parkinson's disease, amyotrophic lateral sclerosis, demyelination, multiple sclerosis, encephalomyelitis, leukoencephalopathy, viral encephalitis, epilepsy, stroke, traumatic brain and spinal cord injury, atherosclerosis, asthma and allergic inflammation, cryopyrin-associated periodic syndromes (CAPS), gout, inflammatory bowel disease (IBD), non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, hidradenitis suppurativa (HS), rheumatoid arthritis, acute kidney disease, chronic kidney disease, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

In yet another embodiment, the disease, disorder or abnormality is selected from: Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury; preferably the disorder is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

In yet another embodiment, the disease, disorder or abnormality is preferably an inflammatory disease, disorder or abnormality; or an autoimmune disease, disorder or abnormality; or a disease, disorder or abnormality of the skin (such as, for example, but not limited to, psoriasis, acne, eczema, alopecia areata, or actinic keratosis); or a disease, disorder or abnormality of the cardiovascular system; or a disease, disorder, or abnormality such as a cancer, a tumor or a malignancy; or a disease, disorder or abnormality of the renal system; a disease, disorder or abnormality of the gastrointestinal tract; a disease, disorder or abnormality of the respiratory system; or a disease, disorder or abnormality of the endocrine system; or a disease, disorder or abnormality of the central nervous system (CNS); or a disease, disorder or abnormality of the liver.

### Definitions

Within the meaning of the present application the following definitions apply unless specified otherwise, and when appropriate, terms used in the singular will also include the plural and vice versa:

“Alkyl” refers to a saturated straight or branched organic moiety consisting of carbon and hydrogen atoms. Examples of suitable alkyl groups have 1 to 6 carbon atoms, preferably 1 to 4 carbon atoms, more preferably 1 to 3 carbon atoms, and (as appropriate) include methyl, ethyl, propyl, isopropyl, n-butyl, t-butyl, and isobutyl. The term “C₁-C₆alkyl” refers to an alkyl group having 1 to 6 carbon atoms. The terms “C₁-C₄alkyl”, “C₁-C₆alkyl”, or “C₁alkyl” are to be construed accordingly.

“Hal”, “halo” or “halogen” refers to F, Cl, Br, and I. Preferably halogen is F or Cl. More preferably, halogen is Cl. Even more preferably, halogen is F.

“—O—C₁-C₆alkyl” where “C₁-C₆alkyl” is as generally defined above. Examples of “—O—C₁-C₆alkyl” include, but are not limited to methoxy, ethoxy, propoxy, isopropoxy, pentoxy, and hexoxy.

“C₁-C₆alkyl-OH” refers to a C₁-C₆alkyl radical as defined above, wherein one of the hydrogen atoms of the C₁-C₆alkyl radical is replaced by “OH”. Examples of “C₁-C₆alkyl-OH” include, but are not limited to, hydroxy-methyl, 2-hydroxy-ethyl, 2-hydroxy-propyl, 3-hydroxy-ethyl, and 5-hydroxy-pentyl.

The term “C₃-C₆cycloalkyl” refers to saturated monocyclic hydrocarbyl groups having 3 to 6 carbon atoms. The terms “C₅-C₆cycloalkyl” is to be construed accordingly. Examples include, but are not limited to cyclopropyl, cyclobutyl, cyclopentyl, and cyclohexyl.

“5- or 6-membered heterocycloalkyl” refers to a stable 5- or 6-membered non-aromatic monocyclic ring radical which comprises 1 or 2 heteroatoms independently selected from nitrogen, oxygen and sulfur. Preferably, the 5- or 6-membered heterocycloalkyl comprises 1 or 2 heteroatoms. More preferably the heteroatom is oxygen or nitrogen. Examples include tetrahydrofuran and oxane.

“Aryl” refers to an aromatic hydrocarbon group having 4 to 8 carbon atoms in the ring portion “3- to 8-membered ring” i.e. four-, five-, six-, seven- or eight-membered ring. Preferably, the term “aryl” refers to an aromatic hydrocarbon group having 6 carbon atoms. Preferably, “aryl” is phenyl.

“Heteroaryl” refers to an aromatic “3- to 8-membered ring” i.e. three-, four-, five-, six-, seven- or eight-membered ring, wherein one or two of the carbon atoms in the ring have been replaced by heteroatoms which are selected from O, N and S. Preferably, “heteroaryl” refers to an aromatic 5- to 8-membered ring. More preferably, “heteroaryl” refers to an aromatic 5- to 6-membered heteroaromatic monocyclic ring radical. The heteroaryl radical may be bonded via a carbon atom or heteroatom. Examples of heteroaryl include, but are not limited to, thiophene, furan, thiazole, isothiazole, oxazole, and isoxazole.

“Optionally substituted” in reference to a certain group refers to said group as to optionally be substituted with one or more substituents (i.e. the substituent may be present or not).

Unless specified otherwise, the term “compound of the present invention” refers to compounds of formula (I) or (II), as disclosed herein, or sub-formulae thereof, as disclosed herein, or stereoisomers thereof, or racemic mixtures thereof, or tautomers thereof, or polymorphs thereof, or pharmaceutically acceptable salts thereof, or prodrugs thereof, or hydrates thereof, or solvates thereof. Compounds of the present invention having one or more optically active carbons can exist as racemates and racemic mixtures (including mixtures in all ratios), stereoisomers (including diastereomeric mixtures and individual diastereomers, enantiomeric mixtures and single enantiomers, mixtures of conformers and single conformers), tautomers, atropisomers, and rotamers. All isomeric forms are included in the present invention. Compounds described in this invention containing olefinic double bonds include E and Z geometric isomers. Also included in this invention are all pharmaceutically acceptable salts, prodrugs, hydrates and solvates of compounds of formula (I) or (II).

Tautomers are isomers of a compound which differ only in the position of the protons and electrons. The skeleton of the compound is unchanged. Common tautomeric pairs include: ketone-enol (H—O—C═CH⇄O═C—CH₂), enamine-imine (H₂N—C═N⇄HN═C—NH).

Solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric acid and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, PA, 1990, p. 1445, the disclosure of which is hereby incorporated by reference.

The compounds of the present invention, as defined herein, can also be provided in the form of a prodrug, namely a compound which is metabolized in vivo to the active metabolite. As used hereinafter in the description of the invention and in the claims, the term “prodrug” means any covalently bonded compound which releases the active parent pharmaceutical due to in vivo biotransformation. The reference by Goodman and Gilman (The Pharmacological Basis of Therapeutics, 8 ed, McGraw-Hill, Int. Ed. 1992, “Biotransformation of Drugs”, p 13-15) describing prodrugs generally is hereby incorporated herein by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

A used herein, the terms “patient” or “subject” mentioned in the present invention typically refer to an animal, particularly a mammal (e.g. rabbits, rats, dogs, mice, guinea pigs, pigs), more particularly primates (e.g. humans, male or female). In certain embodiments, the subject is a human.

“NLRP3” as used herein refers to NOD-like receptor (NLR) family, pyrin-domain containing protein 3 component of inflammasome. Inflammasomes are intracellular supramolecular complexes comprising a sensor molecule, the adaptor apoptosis-associated speck-like protein containing a CARD (ASC) and the effector protease caspase 1. Upon activation of the inflammasome sensor molecule, ASC self-associates into a helical fibrillary assembly resulting in formation of the so-called ASC speck or pyroptosome, which acts as a molecular platform for the activation of pro-caspase 1 via proximity-induced autocatalytic activation. Active caspase 1 triggers the activation and release of interleukin-1 (IL-1) family proteins and enables the non-conventional secretion of numerous cytosolic proteins. Among the pro-inflammatory mediators released upon NLRP3 activation are IL-1 beta (3), IL-18, high-mobility group protein B1 (HMGB1), leukotrienes and prostaglandins.

NLRP3 inflammasome pathway activation is an important driver of inflammation interacting with the different cytokine pathways shaping the immune response to infection and injury. Formation of some pro-inflammatory cytokines is triggered by NLRP3 inflammasome pathway activation.

The terms “inhibit”, “inhibition” or “inhibiting” refer to the reduction or suppression of a given condition, symptom, or disorder, or disease, or abnormality which is responsive to the modulation of a component of the NLRP3 inflammasome pathway, or a significant decrease in the baseline activity of a biological activity or process.

The terms “treat”, “treating” or “treatment” of any disease, disorder or abnormality refer to alleviating or ameliorating or modulating the disease or disorder or abnormality (i.e., slowing or arresting the development of the disease, disorder or abnormality or at least one of the clinical symptoms thereof); or alleviating or ameliorating or modulating at least one physical parameter or biomarker associated with the disease or disorder or abnormality, including those which may not be discernible to the subject (e.g., patient).

The terms “prevent”, “preventing” or “prevention” of any disease or disorder or abnormality which is responsive to the modulation of a component of the NLRP3 inflammasome pathway refer to the prophylactic treatment of the disease or disorder or abnormality; or delaying the onset or progression of the disease or disorder.

The term “in need of” a treatment if such subject would benefit biologically, medically or in quality of life from such treatment.

As used herein, “modulation” refers to alteration, e.g., up-regulation, down-regulation, increase or decrease, preferably decrease.

| Abbreviation | Meaning |
|---|---|
| NALP1-14 | NACHT Leucine-rich-repeat Protein 1-14 (it's a |
| | synonym of NLRP) |
| IPAF | Ice Protease-Activating Factor |
| NAIP | Neuronal Apoptosis Inhibitory Protein |
| ASC | Apoptosis-associated Speck-like protein containing a |
| | CARD |
| nucleotide- | NACHT: NAIP (neuronal apoptosis inhibitory |
| binding NACHT | protein), CIITA (MHC class II transcription |
| domain | activator), HET-E (incompatibility locus protein |
| | from Podospora anserina) and TP1 (telomerase- |
| | associated protein) |
| IL | Interleukin |
| TNF-alpha | Tumor Necrosis Factor - alpha |

The definitions and preferred definitions given in the “Definition”-section apply to all of the embodiments described herein unless stated otherwise.

The compounds of the present invention can be synthesized by those skilled in the art by using commonly known preparation steps, for instance those of the general methods shown in the following schemes. These methods are only given for illustrative purposes and should not to be construed as limiting.

General Synthetic Scheme for the Preparation of Compounds of this Invention:

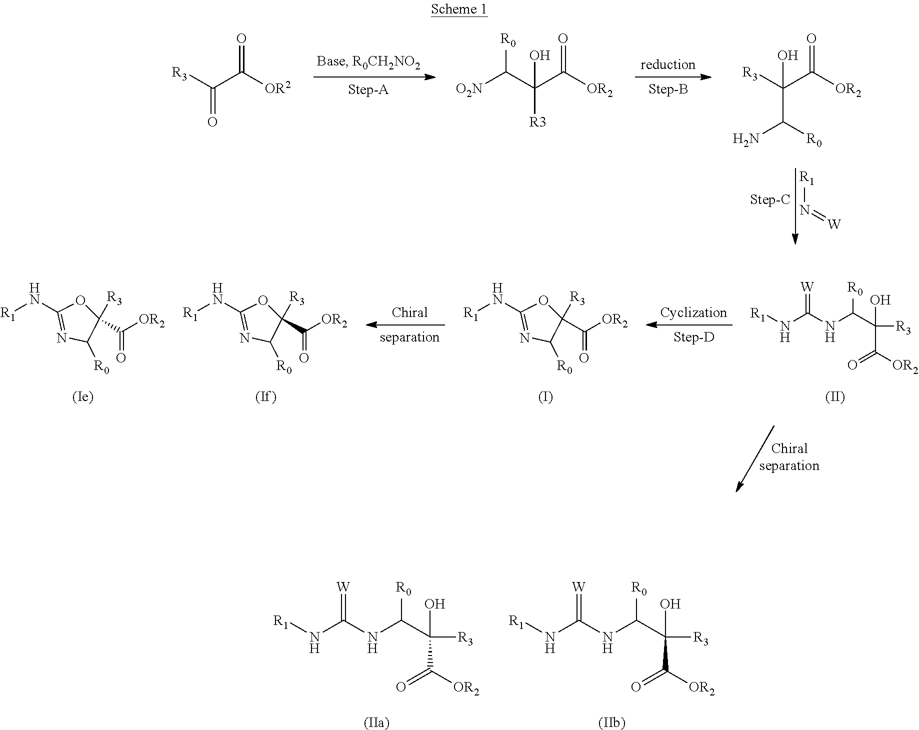

From a commercially available arylketoester, a nitro derivative was synthesized using a nitroalkane such as nitromethane with a suitable base and solvent. Reduction of nitro was then achieved either by hydrogen with an appropriate catalyst or by using a metal under acidic conditions such iron or zinc in acetic acid. Subsequently, a thiourea or urea derivative was obtained by treating the amine intermediate with isothiocyanate or isocyanate optionally in the presence of a base. Finally, the cyclization was carried out as known in the art such as, for example, using N-(3-dimethylaminopropyl)-N′-ethylcarbodiimide (EDC) with a base, Mukaiyama reagent with a base, iodomethane with a base or TsCI with a base. Alternatively, R² functionality could be also introduced at the end of the synthesis by a two-step strategy by saponification/esterification procedures. Ultimately, the enantiomers could be separated by chiral supercritical fluid chromatography (SFC) to obtain the desired single enantiomers.

### EXAMPLES

The disclosure is further illustrated by the following examples and synthesis schemes, which are not to be construed as limiting the scope of the specific procedures herein described. It is understood that the examples are provided to illustrate certain embodiments and that no limitation to the scope of this disclosure is intended thereby.

| | |
|---|---|
| ACN | Acetonitrile |
| Ac₂O | Acetic anhydride |
| AgF | Silver fluoride |
| Boc₂O | di-tert-butyl dicarbonate (Boc-anhydride) |
| CS₂ | Carbon disulfide |
| DCM | Dichloromethane |
| DMAP | 4-Dimethylaminopyridine |
| EDC•HCl | N-(3-Dimethylaminopropyl)-N′- |
| | ethylcarbodiimid-hydrochloride |
| EtOAc | Ethyl acetate |
| EtOH | Ethanol |
| Et₃SiH | Triethylsilane |
| HCl | Hydrochloric acid |
| H₂SO₄ | Sulfuric acid |
| HNO₃ | Nitric acid |
| HPLC | High performance liquid chromatography |
| K₂CO₃ | Potassium carbonate |
| LCMS | Liquid Chromatograph-Mass Spectrometry |
| LiOH•H₂O | Lithium hydroxide monohydrate |
| MeOH | Methanol |
| N₂ | Nitrogen |
| NaBH₄ | Sodium borohydride |
| Na₂CO₃ | Sodium carbonate |
| NaOH | Sodium hydroxide |
| Na₂SO₄ | Sodium sulfate |
| NBS | N-Bromosuccinimide |
| NH₄Cl | Ammonium chloride |
| NEt₃ | Triethylamine |
| Pd/C | Palladium on carbon |
| Pd(dppf)Cl₂ or | 1,1′-Bis(diphenylphosphino)ferrocene]dichloropalla- |
| Pd(dppf)Cl₂ × | dium(II), complex with dichloromethane |
| CH₂Cl₂ | |
| Pd(OAc)₂ | Palladium (II) acetate |
| Pd(PPh₃)₄ | Tetrakis(triphenylphosphine)palladium(0) |
| rt or RT | Room temperature |
| SiO₂ | Silica gel |
| SFC | Supercritical fluid chromatography |
| t-BuNO₂ | Tert-Butyl nitrite |
| TFA | Trifluoroacetic acid |
| THF | Tetrahydrofuran |
| TLC | Thin layer chromatography |
| TMSCF₃ | Trifluoromethyltrimethylsilane |
| SeO₂ | Selenium dioxide |
| SM | Starting material |

All reagents and solvents were obtained from commercial sources and used without further purification. ¹H-NMR spectra were recorded on Bruker 400 MHz-AVANCE III HD NMR, Bruker 500 MHz-AVANCE III HD NMR spectrometers or Spinsolve 80 Mhz in deuterated solvents. Chemical shifts (δ) are reported in parts per million and coupling constants (J values) in hertz. Spin multiplicities are indicated by the following symbols: s (singlet), d (doublet), t (triplet), q (quartet), m (multiplet), bs (broad singlet). Mass spectra were obtained on a Water ACQUITY SQD2 UPLC/MS system. GC-MS data were collected using an Agilent 7890B gas chromatograph and 5977A mass spectrometer. Chromatography was performed using silica gel (Acme: Silica gel 60, 0.063-0.2 mm) and suitable solvents as indicated in specific examples. Flash purification was conducted with a Biotage Isolera one or Reveleris X₂ with KP—NH SNAP cartridges (Biotage) or Reveleris silica cartridges (Grace) and the solvent gradient indicated in specific examples. Thin layer chromatography (TLC) was carried out on silica gel plates (Merck) with UV detection.

### Intermediate 1:4-(5-fluoro-3-isopropyl-2-isothiocyanatophenyl)-2-methoxypyridine

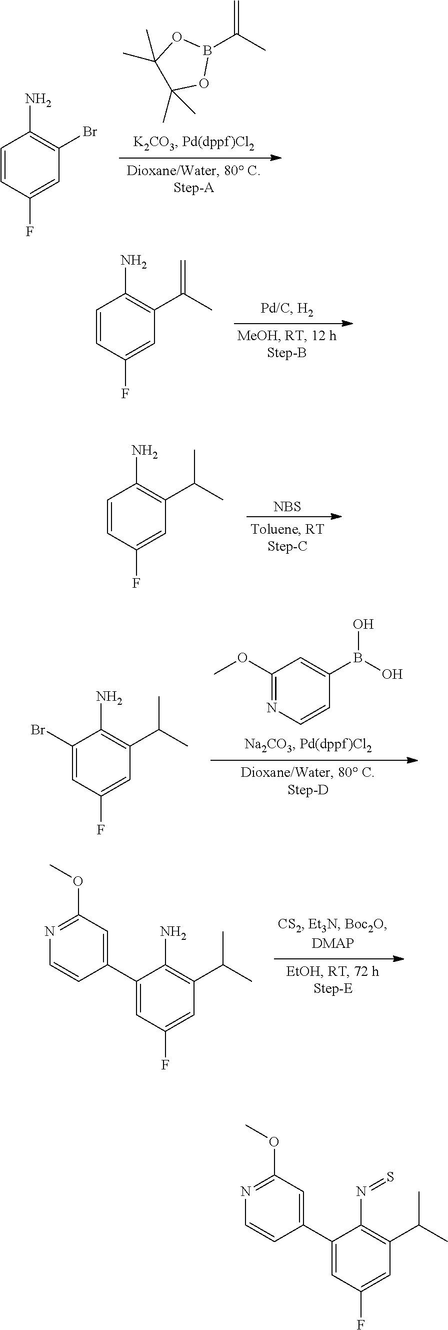

### Step A

To a stirred solution of 2-bromo-4-fluoroaniline (9.0 g, 47.365 mmol) and 4,4,5,5-tetramethyl-2-(prop-1-en-2-yl)-1,3,2-dioxaborolane (8.35 g, 49.73 mmol) in 1,4-dioxane (45 ml) and water (9 ml) was added K₂CO₃ (13.09 g, 94.73 mmol) at room temperature in a sealed tube and the resulting mixture was purged with N₂ for 10 minutes. After adding Pd(dppf)Cl₂xCH₂Cl₂ (1.932 g, 2.368 mmol), the reaction mixture was heated to 80° C. After 16 hours stirring, the reaction mixture was diluted with water (100 ml) and extracted with ethyl acetate (2×100 ml). The combined organic layers were dried over Na₂SO₄, filtered and concentrated under reduced pressure. The resulting crude residue was purified by flash chromatography (SiO₂ column, 0-10% ethyl acetate in hexane) to afford 4-fluoro-2-(prop-1-en-2-yl) aniline as a yellow oil. (5.0 g, 70%). MS: 152.00 [M+H]⁺.

### Step B

To a solution of the compound from step-A (5.0 g, 33.112 mmol) in MeOH (100 ml) was added 10% Pd/C (2.0 g) and the resulting mixture was hydrogenated at 50 psi for 12 hours. The reaction mixture was filtered through celite, and the filtrate was evaporated under reduced pressure to afford 4-fluoro-2-isopropylaniline as a black oil (5.0 g, 98%). MS: 154.04 [M+H]⁺.

### Step C

To a stirred solution of the compound obtained from step-B (7.0 g, 45.751 mmol) in toluene (105 ml) was added N-bromosuccinimide (8.142 g, 45.751 mmol) portionwise at 0° C., the resulting mixture was stirred at 0° C. under N₂ atmosphere for 30 minutes. After complete consumption of the starting material, the reaction mixture was quenched with ice water (100 ml) and extracted with ethyl acetate (2×100 ml). The combined organic layers were dried over Na₂SO₄ and concentrated under reduced pressure. The resulting crude residue was purified by flash chromatography (SiO₂ column, 100% hexane) to afford 2-bromo-4-fluoro-6-isopropylaniline as a brown oil (8.0 g, 75%).

MS: 232.03 [M+H]⁺.

### Step-D

To a stirred solution of compound from step-C (8.0 g, 34.482 mmol) and (2-methoxypyridin-4-yl) boronic acid (8.35 g, 49.73 mmol) in 1,4-dioxane (80 ml) and water (16 ml) was added Na₂CO₃ (9.13 g, 86.205 mmol) followed by Pd(dppf)Cl₂xCH₂Cl₂ (2.81 g, 3.448 mmol) at room temperature and the resulting mixture was heated to 80° C. After stirring for 12 hours, the reaction mixture was filtered through celite, and the filtrate was diluted with water (100 ml) and extracted with ethyl acetate (2×100 ml). The combined organic layers were dried over Na₂SO₄, filtered, and concentrated under reduced pressure. The resulting crude residue was purified by flash chromatography (SiO₂ column, 0-10% ethyl acetate in hexane) to afford 4-fluoro-2-isopropyl-6-(2-methoxypyridin-4-yl) aniline as an off-white solid (5.7 g, 63.5%). ¹H-NMR (500 MHz DMSO-d6): δ=8.27-8.21 (dd, 1H), 7.03-7.02 (dd, 1H), 6.94-6.92 (dd, 1H), 6.82-6.82 (s, 1H), 6.74-6.72 (dd, 1H), 4.47 (s, 2H), 3.89 (s, 3H), 3.08-3.03 (m, 1H), 1.18-1.16 (d, 6H). MS: 261.44 [M+H]⁺.

### Step-E

A solution of compound from step-D (500 mg, 1.92 mmol) in CS₂ (1.16 ml, 19.20 mmol) and NEt₃ (0.53 ml, 3.84 mmol) was stirred at 0° C. for 30 minutes, then a solution of Boc₂O (0.88 ml, 3.84 mmol) in EtOH (3 ml) was added dropwise at 0° C., followed by a catalytic amount of DMAP (10 mg). The resulting mixture was left to warm up to RT and stirred for 72 hours. After complete consumption of the starting material, the reaction mixture was concentrated under reduced pressure. The crude residue was purified by flash chromatography (SiO₂ column, 0-10% ethyl acetate in hexane) to afford 4-(5-fluoro-3-isopropyl-2-isothiocyanatophenyl)-2-methoxypyridine (0.380 g, 65.3%). ¹H-NMR (400 MHz CDCl₃): δ=8.28-8.26 (d, 1H), 7.05-7.02 (d, 1H), 6.95-6.89 (m, 2H), 6.79 (bs, 1H), 4.15-4.09 (m, 1H), 4.00 (s, 3H), 3.32-3.28 (m, 1H), 1.30-1.25 (m, 6H). MS: 303.20 [M+H]⁺.

### Intermediate 2:4-(4-isothiocyanato-2,3-dihydro-1H-inden-5-yl)-2-methoxypyridine

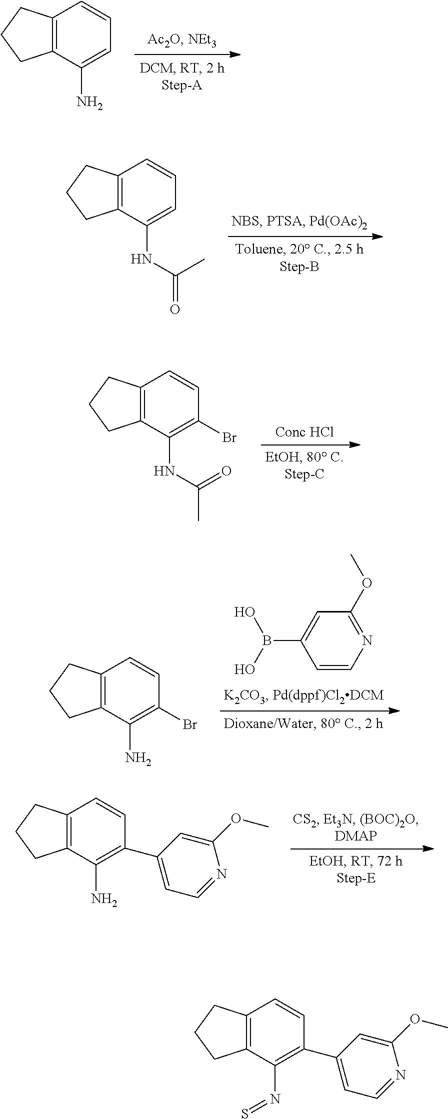

### Step A

To a stirred solution of 2,3-dihydro-1H-inden-4-amine (25.0 g, 187.70 mmol) and trimethylamine (32.5 ml, 244.01 mmol) in DCM (400 ml) was added acetic anhydride (20 ml, 225.24 mmol) in one portion at 0° C. and the reaction mixture was allowed to slowly warm up to room temperature. After 2 hours stirring, the reaction mixture was diluted with water (500 ml) and extracted with DCM (2×500 ml), the combined organic layers were dried over Na₂SO₄ and concentrated under reduced pressure to afford N-(2,3-dihydro-1H-inden-4-yl) acetamide as an off-white solid (32.5 g, 99%). ¹H-NMR (400 MHz CDCl₃): δ=7.74-7.72 (d, 1H), 7.17-7.13 (t, 1H), 7.03-7.01 (d, 1H), 6.95 (brs, 1H), 2.97-2.93 (t, 2H), 2.83-2.79 (t, 2H), 2.19 (s, 3H), 2.15-2.07 (m, 2H). MS: 176.22 [M+H]⁺.

### Step B

To a stirred solution of compound from step-A (30.0 g, 0.171 mol) in toluene (400 ml) was added p-toluenesulfonic acid (17.93 g, 0.094 mol) followed by Pd(OAc) 2 (1.91 g, 0.008 mol) at room temperature. After stirring for 30 minutes, N-bromosuccinimide (33.569 g, 0.188 mol) was added, and the reaction mixture was further stirred for 2 hours. The reaction mixture was diluted with ice water (500 ml) and extracted with ethyl acetate (2×500 ml). The combined organic layers were dried over Na₂SO₄ and concentrated under reduced pressure. The crude residue was triturated with diethyl ether (200 ml), filtered, and dried to afford N-(5-bromo-2,3-dihydro-1H-inden-4-yl) acetamide as an off-white solid (33.0 g, 76%). ¹H-NMR (400 MHz CDCl₃): δ=7.36-7.34 (d, 1H), 7.01-6.98 (d, 1H), 2.92-2.85 (m, 4H), 2.22 (s, 3H), 2.10-2.03 (m, 2H). MS: 254.19 [M+H]⁺.

### Step C

To a stirred solution of compound from step-B (33.0 g, 129.92 mmol) in EtOH (200 ml) was added concentrated HCl (230 ml) at 0° C.; the mixture was then heated 80° C. After stirring for 48 hours, the reaction mixture was cooled to 0° C. The solid formed was filtered through a Buchner funnel, washed with ice cold water (200 ml) and dried to afford 5-bromo-2,3-dihydro-1H-inden-4-amine as an off-white solid (22.0 g, 80%). ¹H-NMR (500 MHz DMSO-d6): δ=7.12-7.10 (d, 1H), 6.42-6.41 (d, 1H), 2.77-2.75 (t, 2H), 2.72-2.69 (t, 2H), 2.01-1.98 (m, 2H). MS: 212.22 [M+H]⁺.

### Step-D

To a stirred solution of the compound from step-C (5.0 g, 0.0235 mol) and (2-methoxypyridin-4-yl) boronic acid (5.4 g, 0.035 mol) in 1,4-dioxane (120 ml) and water (30 ml) was added K₂CO₃ (6.5 g, 0.0471 mol) in one portion at room temperature and the resulting mixture was degassed by bubbling of N₂ for 10 minutes. Then Pd(dppf)Cl₂xCH₂Cl₂ (0.96 g, 0.0013 mol) was added at room temperature and the mixture was heated to 80° C. After stirring for 3 hours, the reaction mixture was filtered through celite, the filtrate was diluted with water (400 ml) and extracted with ethyl acetate (2×300 ml). The combined organic layers were dried over Na₂SO₄ and concentrated under reduced pressure. The resulting crude residue was purified by flash chromatography (SiO₂ column, 0-40% ethyl acetate in hexane) to afford 5-(2-methoxypyridin-4-yl)-2,3-dihydro-1H-inden-4-amine (4.0 g, 71.4%) as an off-white solid. ¹H-NMR (500 MHz CDCl₃): δ=8.21-8.20 (d, 1H), 7.01-6.99 (d, 1H), 6.98-6.96 (d, 1H), 6.85 (s, 1H), 6.76-6.75 (d, 1H), 3.98 (s, 3H), 3.74 (brs, 2H), 2.97-2.94 (t, 2H), 2.77-2.74 (t, 2H), 2.19-2.14 (m, 2H). MS: 241.36 [M+H]⁺.

### Step-E

A solution of compound from step-D (500 mg, 2.08 mmol) in CS₂ (1.5 ml, 20.82 mmol) and NEt₃ (0.58 ml, 4.16 mmol) was stirred at 0° C. for 30 minutes, then a solution of Boc₂O (0.95 ml, 4.16 mmol) in EtOH (3 ml) was added dropwise at 0° C. followed by a catalytic amount of DMAP (10 mg). The resulting mixture was left to warm up to room temperature and stirred. After 72 hours stirring, the reaction mixture was concentrated under reduced pressure. The crude residue was purified by flash chromatography (SiO₂ column, 0-10% ethyl acetate in hexane) to afford 4-(4-isothiocyanato-2,3-dihydro-1H-inden-5-yl)-2-methoxypyridine (0.3 g, 50.84%). ¹H-NMR (400 MHz CDCl₃): δ=8.24-8.23 (d, 1H), 7.21-7. (d, 1H), 7.16-7.14 (d, 1H), 6.99-6.97 (d, 1H), 6.82 (bs, 1H), 3.98 (s, 3H), 3.11-3.07 (m, 2H), 2.98-3.02 (m, 2H), 2.22-2.14 (m, 2H). MS: 283.17 [M+H]⁺.

### Intermediate 3:4-isothiocyanato-8-methyl-1,2,3,5,6,7-hexahydro-s-indacene

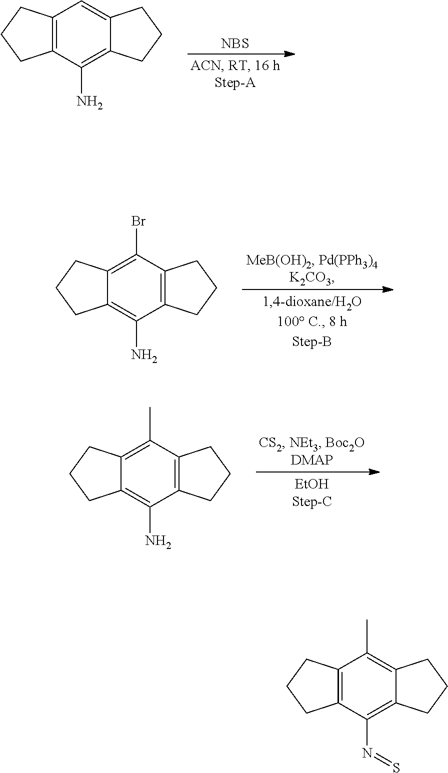

### Step-A:

To a stirred solution of 1,2,3,5,6,7-hexahydro-s-indacen-4-amine (3.0 g, 17.31 mmol) ACN (45 ml), NBS (3.389 g, 19.04 mmol) and NEt₃ (0.53 ml, 3.84 mmol) were added, and the resulting mixture was stirred at room temperature. After 2 hours stirring, the reaction mixture was concentrated under reduced pressure. The crude residue was diluted with ethyl acetate (200 ml) and washed with brine (100 ml). The organic layer was dried over Na₂SO₄, filtered, and evaporated under reduced pressure. The resulting crude material was purified by romatography (SiO₂ column, 2-10% ethyl acetate in hexane) to afford 8-bromo-1,2,3,5,6,7-hexahydro-s-indacen-4-amine (2.9 g, 89.55%). ¹H-NMR (400 MHz CDCl₃): δ=3.44 (s, 2H), 2.92-2.88 (t, 4H), 2.81-2.77 (t, 4H), 2.17-2.09 (m, 4H).

MS: 252.11 [M+H]⁺.

### Step-B:

In a sealed tube were placed compound from step-A (1.5 g, 5.948 mmol), NBS (3.389 g, 19.04 mmol), methyl boronic acid (0.534 g, 8.922 mmol), NEt₃ (0.53 ml, 3.84 mmol), K₂CO₃ (2.46 g, 17.84 mmol) in 1,4-dioxane (25 ml) and H₂O (6 ml); the mixture was purged with N₂ for 10 minutes followed by the addition of Pd(PPh₃)₄ (0.343 g, 0.297 mmol). The resulting mixture was heated at 100 C. After 8 hours stirring, the mixture was diluted with water (30 ml), extracted with ethyl acetate (100 ml). The organic layer was washed with brine (100 ml), dried over Na₂SO₄, filtered, and evaporated under reduced pressure. The resulting crude residue was purified by flash chromatography (SiO₂ column, 0-10% ethyl acetate in hexane) to afford 8-methyl-1,2,3,5,6,7-hexahydro-s-indacen-4-amine (0.9 g, 88.27%). MS: 187.29 [M+H]⁺.

### Step-C:

A solution of compound from step-B (0.100 g, 0.534 mmol) in CS₂ (0.32 ml, 5.347 mmol) and NEt₃ (0.074 ml, 0.534 mmol) was stirred at 0° C. for 30 minutes, then a solution of Boc₂O (1.0 ml, 0.534 mmol) in EtOH (2 ml) was added dropwise at 0° C. followed by a catalytic amount DMAP (2 mg, 0.016 mmol) and the resulting mixture was allowed to warm up to RT. After 5 hours stirring, the mixture was concentrated under reduced pressure to afford 4-isothiocyanato-8-methyl-1,2,3,5,6,7-hexahydro-s-indacene (0.120 g, 91%). MS: 229.34 [M+H]⁺.

### Intermediate 4:4-(7-fluoro-4-isothiocyanato-2,3-dihydro-1H-inden-5-yl)-2-methoxypyridine

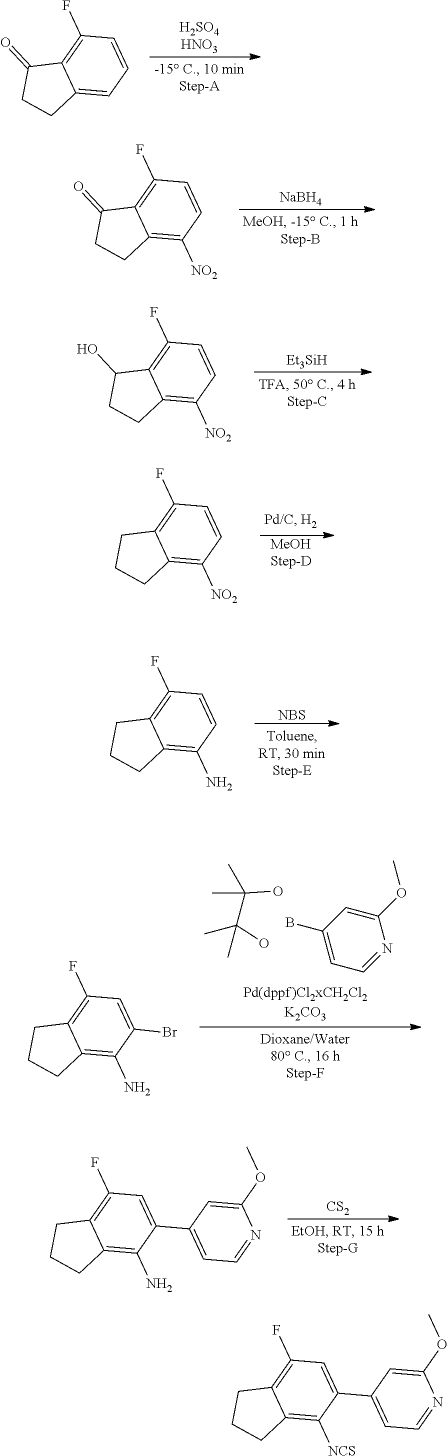

### Step-A:

To a mixture of 7-fluoro-2,3-dihydro-1H-inden-1-one (10 g, 0.0666 mol) in concentrated sulfuric acid (80 ml) at −15° C., was added dropwise a solution of nitric acid (30 ml) in concentrated sulfuric acid (20 ml) and the reaction mixture was stirred at −15° C. for 10 minutes. The mixture was quenched with water (1000 ml) at 0° C. and extracted with EtOAc (3×500 ml). The combined organic phases were dried over anhydrous Na₂SO₄, filtered and concentrated under reduced pressure. The resulting crude residue was purified by flash chromatography (SiO₂ column 230-400 mesh, 0-50% ethyl acetate in petroleum ether) to afford 7-fluoro-4-nitro-2,3-dihydro-1H-inden-1-one (6.2 g, 47%) as a yellow solid.

MS: 196.02 [M+H]⁺.

### Step-B:

To a solution of the compound from Step-A (6 g, 13.95 mmol) in MeOH (60 ml) was added NaBH4 (2.27 g, 61.53 mmol) portionwise and the resulting mixture was stirred at 15° C. After stirring for 1 hour at 15° C., the mixture was poured into water (500 ml) and extracted with EtOAc (2×500 ml). The combined organics were washed with brine (200 ml), dried over anhydrous Na₂SO₄, filtered and concentrated under reduced pressure to afford 7-fluoro-4-nitro-2,3-dihydro-1H-inden-1-ol (7 g, crude) as a brown oil. MS: 197.82 [M+H]⁺.

### Step-C:

To a solution of the compound from Step-B (6 g, 0.03045 mol) in TFA (60 ml) was added triethylsilane (24.5 ml, 0.1522 mol) in one portion. The resulting reaction mixture was heated at 50° C. After 4 hours stirring at 50° C., the mixture was concentrated under reduced pressure and quenched with 1M aqueous NaOH (100 ml). The resulting mixture was extracted with EtOAc (3×100 ml). The combined organics were washed with brine (50 ml), dried over anhydrous Na₂SO₄, filtered and concentrated under reduced pressure to afford fluoro-7-nitro-2,3-dihydro-1H-indene (8 g, crude) as a brown oil.

### Step-D:

To a solution of compound from Step-C (5.5 g, 0.03038 mol) in MeOH (60 ml) under nitrogen atmosphere was added 10% Pd/C (9.6 g, 50% wet) in one portion and the reaction mixture was stirred at 25° C. under H₂ atmosphere. After 12 hours stirring, the reaction mixture was filtered and the filtrate was concentrated under reduced pressure. The residue was purified by column chromatography (SiO₂ column 230-400 mesh, 0-60% ethyl acetate in petroleum ether) to afford 7-fluoro-2,3-dihydro-1H-inden-4-amine (4.5 g, 97%) as a brown solid. MS: 152.02 [M+H]⁺.

### Step-E:

To a stirred solution of compound from Step-D (4.5 g, 0.02980 mol) in toluene (45 ml) was added NBS (10.5 g, 0.02980 mol) at 25° C. in a single portion. The resulting dark brown mixture was stirred at 25° C. After 30 minutes, the reaction mixture was quenched with saturated aqueous Na₂SO₃ solution (150 ml) and extracted with EtOAc (2×150 ml). The combined organic phases were washed with brine (100 ml), dried over anhydrous Na₂SO₄, filtered and concentrated under reduced pressure. The residue was purified by column chromatography (SiO₂ column 230-400 mesh, 0-20% ethyl acetate in petroleum ether) to afford 5-bromo-7-fluoro-2,3-dihydro-1H-inden-4-amine (4 g, 58%) as a brown solid. MS: 230.06 [M+H]⁺.

### Step-F:

To a stirred solution of compound from Step-E (4 g, 0.01754 mol) in dioxane (40 ml) and water (10 ml) was added K₂CO₃ (4.84 g, 0.03508 mol) and the resulting mixture was degassed with nitrogen for 10 minutes. To this mixture, Pd(dppf)Cl₂xCH₂Cl₂ complex (816 mg, 0.00877 mol) was added in one portion under N₂ atmosphere. Then the reaction mixture was heated to 80° C. After 16 hours stirring at 80° C., the mixture was quenched with water (50 ml) and extracted with EtOAc (3×50 ml). The combined organics were washed with brine (30 ml), dried over anhydrous Na₂SO₄, filtered and concentrated under reduced pressure. The residue was purified by column chromatography (SiO₂ column 230-400 mesh, 0-30% ethyl acetate in petroleum ether) to afford 7-fluoro-5-(2-methoxypyridin-4-yl)-2,3-dihydro-1H-inden-4-amine (4 g, 91%) as a brown solid. MS: 259.24 [M+H]⁺.

### Step-G:

To a solution of compound from Step-F (4 g, 0.01550 mol) in EtOH (40 ml) were added CS₂ (2.3 ml, 0.04651 mmol) and Et₃N (6.4 ml, 0.04651 mol) and the reaction mixture was stirred at room temperature. After 16 hours stirring, the reaction mixture was concentrated in vacuum. The crude compound was purified by silica gel column chromatography (SiO₂ column 230-400 mesh, 0-30% ethyl acetate in petroleum ether) to afford 4-(7-fluoro-4-isothiocyanato-2,3-dihydro-1H-inden-5-yl)-2-methoxypyridine (1.1 g, 24%) as a light brown solid. MS: 301.29 [M+H]⁺.

### Intermediate 5:4-isothiocyanato-8-(trifluoromethyl)-1,2,3,5,6,7-hexahydro-s-indacene

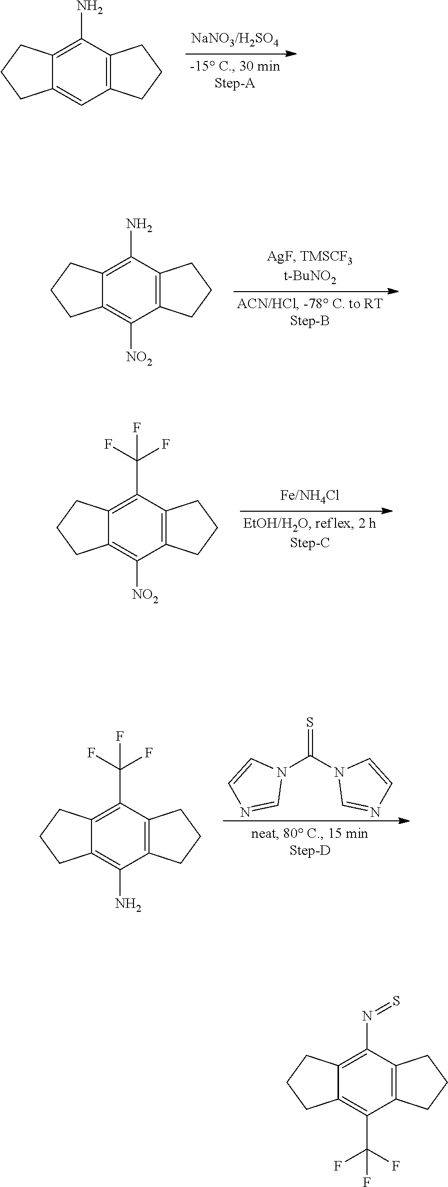 [SMILES: Nc1c2c(cc3c1CCC3)CCC2 ; Nc1c2c(c([N+](=O)[O-])c3c1CCC3)CCC2 ; O=[N+]([O-])c1c2c(c(C(F)(F)F)c3c1CCC3)CCC2 ; Nc1c2c(c(C(F)(F)F)c3c1CCC3)CCC2 ; S=C(n1ccnc1)n1ccnc1 ; FC(F)(F)c1c2c(c(N=S)c3c1CCC3)CCC2]

### Step-A:

1,2,3,5,6,7-hexahydro-s-indacen-4-amine (2.0 g, 11.54 mmol) was added to a cooled solution of H₂SO₄ (10 ml) at −15° C. followed by the addition of NaNO₃ (0.981 g, 11.543 mmol) portionwise at −15° C. and the resulting mixture was stirred. After 30 minutes stirring at −15° C., the reaction mixture was quenched with ice water (20 ml) and stirred for an additional 15 minutes resulting in precipitation of a brown solid. The solid was filtered off and washed with ice water (20 ml) and dried thoroughly under vacuum to afford 8-nitro-1,2,3,5,6,7-hexahydro-s-indacen-4-amine (1.6 g, 63.54%) as a brown solid.

¹H-NMR (400 MHz CDCl₃): δ=7.26 (s, 2H), 4.05 (brs, 4H), 2.86-2.84 (m, 2H), 2.11-2.09 (m, 2H), 1.56 (brs, 4H) MS: 219.12 [M+H]⁺.

### Step-B:

To a stirred solution of silver (I) fluoride (4.359 g, 34.362 mmol) in ACN (30 ml) was added trifluoromethyltrimethylsilane (5.08 ml, 34.36 mmol) dropwise at −78° C. and the resulting mixture was stirred for 15 minutes. The reaction mixture was allowed to warm up to room temperature and stirred for 30 minutes until a dark grey suspension was observed. To a solution of compound from step-A (1.5 g, 6.87 mmol) in ACN (30 ml) was added 11.6N HCl (1.184 ml, 13.745 mmol) at −15° C. followed by dropwise addition of t-BuNO₂ (0.906 ml, 7.559 mmol) at −15° C. and stirred for 15 minutes. To this was added the previously prepared AgCF₃ suspension at −78° C. The resulting reaction mixture was left to gradually warm up to room temperature. After stirring for 6 hours, the resulting residue was diluted with ethyl acetate (30 ml), filtered through a celite pad and washed with EtOAc (60 ml). The filtrate was washed with brine (50 ml). The organic layer was dried over Na₂SO₄, filtered and evaporated under reduced pressure. The crude compound was purified by flash chromatography (SiO₂ column, 0-30% ethyl acetate in petroleum ether) to afford 4-nitro-8-(trifluoromethyl)-1,2,3,5,6,7-hexahydro-s-indacene (0.25 g, 13%) as a colorless liquid. MS: 271.1 [M]⁺.

### Step-C:

To a stirred solution of the compound obtained from step-B (0.25 g, 0.921 mmol) in EtOH (10 ml) and H₂O (3 ml) was added NH₄Cl (0.295 g, 5.530 mmol) followed by iron powder (0.257 g, 4.608 mmol) in one portion at room temperature and the resulting mixture was refluxed for 2 hours. The reaction mixture was diluted with ethyl acetate (20 ml) and filtered through a celite pad and washed with EtOAc (30 ml). The filtrate was washed with brine (30 ml). The organic layer was dried over Na₂SO₄, filtered and evaporated under reduced pressure to afford crude 8-(trifluoromethyl)-1,2,3,5,6,7-hexahydro-s-indacen-4-amine (0.20 g, crude) as a colorless liquid. MS: 242.09 [M+H]⁺.

### Step-D:

To compound obtained from step-C (0.180 g, 0.746 mmol) was added 1,1′-thiocarbonyldiimidazole (0.199 g, 1.119 mmol) in one portion at room temperature and the resulting mixture was heated to 80° C. After stirring for 30 minutes at 80° C., the reaction mixture was diluted with 10% diethyl ether in n-pentane (20 ml) and stirred for 5 minutes. The resulting mixture was filtered through a silica bed, washed with 10% diethyl ether in n-pentane (40 ml) and the filtrate was evaporated under reduced pressure to afford 4-isothiocyanato-8-(trifluoromethyl)-1,2,3,5,6,7-hexahydro-s-indacene (0.22 g, crude) as a colorless gum. MS: 283.1 [M]⁺.

### Intermediate 6:4-fluoro-7-isothiocyanato-2,3-dihydro-1H-indene

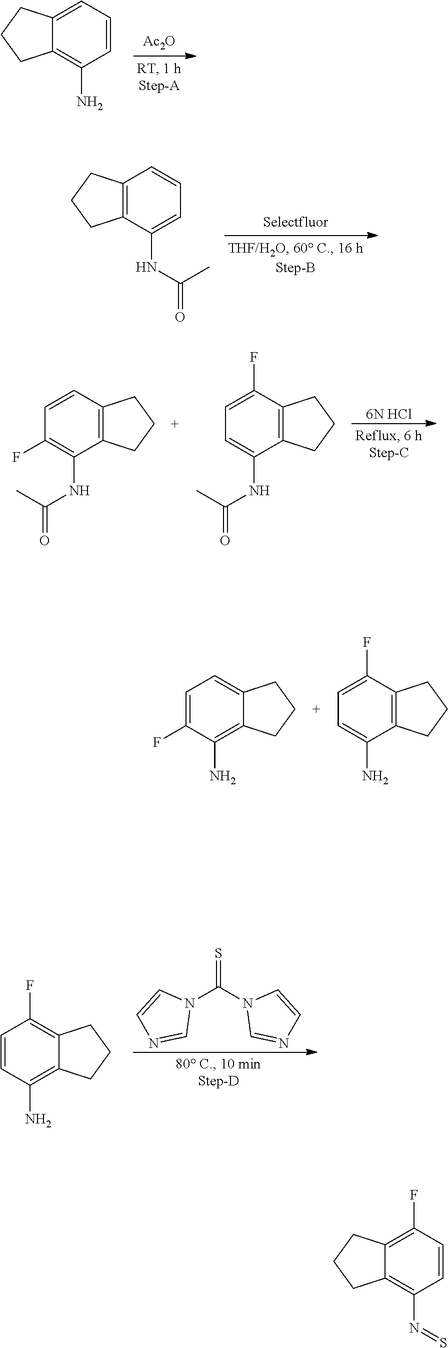

### Step-A

A solution of 2,3-dihydro-1H-inden-4-amine (15 g, 112.60 mmol) in acetic anhydride (50 ml) was stirred at room temperature. After 1 hour stirring, the reaction mixture was concentrated under reduced pressure to afford N-(2,3-dihydro-1H-inden-4-yl) acetamide (17 g, 86.2%) as an off-white solid.

¹H-NMR (400 MHz CDCl₃): δ=7.73-7.71 (d, 1H), 7.16-7.12 (t, 1H), 7.03-7.01 (d, 2H), 2.96-2.92 (t, 2H), 2.82-2.78 (t, 2H), 2.18 (s, 3H), 2.14-2.06 (m, 2H). MS: 176.06 [M+H]⁺.

### Step-B

To a stirred solution of the compound from Step-A (16 g, 91.31 mmol) in THF (160 ml) and water (160 ml) was added Selectfluor (64.6 g, 182.62 mmol) at room temperature and the resulting mixture was heated to 60° C. After stirring for 16 hours at 60° C., the reaction mixture was diluted with water (500 ml) and extracted with EtOAc (2×500 ml). The combined organic layers were washed with brine (100 ml), dried over anhydrous Na₂SO₄, filtered and concentrated under reduced pressure to get the crude mixture (20 g, crude) of N-(5-fluoro-2,3-dihydro-1H-inden-4-yl) acetamide and N-(7-fluoro-2,3-dihydro-1H-inden-4-yl) acetamide as a brown gummy solid.

MS: 194.07 [M+H]+.

### Step-C

A solution of the mixture of compounds from Step-B (20 g, 103.51 mmol) in 6N HCl (100 ml) was stirred at 100° C. After stirring for 6 hours at 100° C., the reaction mixture was basified with aqueous NaHCO₃ at 0° C. The resulting mixture was stirred for 10 minutes and was then extracted with EtOAc (2×500 ml) and water (100 ml). The combined organic layers were washed with brine solution (100 ml), dried over anhydrous Na₂SO₄, filtered and concentrated under reduced pressure. The crude mixture was purified by flash chromatography (SiO₂ column, 0-10% ethyl acetate in petroleum ether) to afford 5-fluoro-2,3-dihydro-1H-inden-4-amine (1.2 g, 9.2%) as the first eluting isomer.

¹H-NMR (400 MHz DMSO-d6): δ=6.77-6.72 (m, 1H), 6.39-6.32 (m, 1H), 4.69 (s, 2H), 2.79-2.72 (t, 2H), 2.69-2.65 (t, 2H), 2.02-1.95 (m, 2H). MS: 151.98 [M+H]+.

7-fluoro-2,3-dihydro-1H-inden-4-amine (1 g, 7.7%) was obtained as the second eluting isomer. ¹HNMR (400 MHz DMSO-d6): δ=6.65-6.60 (t, 1H), 6.36-6.33 (m, 1H), 4.837 (s, 2H), 2.82-2.78 (t, 2H), 2.66-2.63 (t, 2H), 2.04-1.97 (m, 2H). MS: 151.98 [(M+H)]+.

### Step-D

A stirred solution of 7-fluoro-2,3-dihydro-1H-inden-4-amine obtained from Step-C (1.0 g, 6.614 mmol) and 1,1′-thiocarbonyldiimidazole (1.76 g, 9.922 mmol) was heated to 80° C. in a sealed tube. After 10 minutes stirring at 80° C., the reaction mixture was diluted with DCM (10 ml) and concentrated under reduced pressure. The crude product was purified by flash chromatography (SiO₂ column, 100% petroleum ether) to afford 4-fluoro-7-isothiocyanato-2,3-dihydro-1H-indene (1.0 g, 78.7%) as a yellow liquid.

¹H-NMR (400 MHz CDCl₃): δ=6.97-6.94 (t, 1H), 6.82-6.78 (t, 1H), 2.05-2.01 (t, 2H), 2.99-2.95 (t, 2H), 2.20-2.12 (m, 2H). MS: 193.1 [M]⁺.

### Example 1: ethyl 2-{[4-fluoro-2-(2-methoxypyridin-4-yl)-6-(propan-2-yl)phenyl]amino}-5-(1,2-oxazol-3-yl)-4,5-dihydro-1,3-oxazole-5-carboxylate

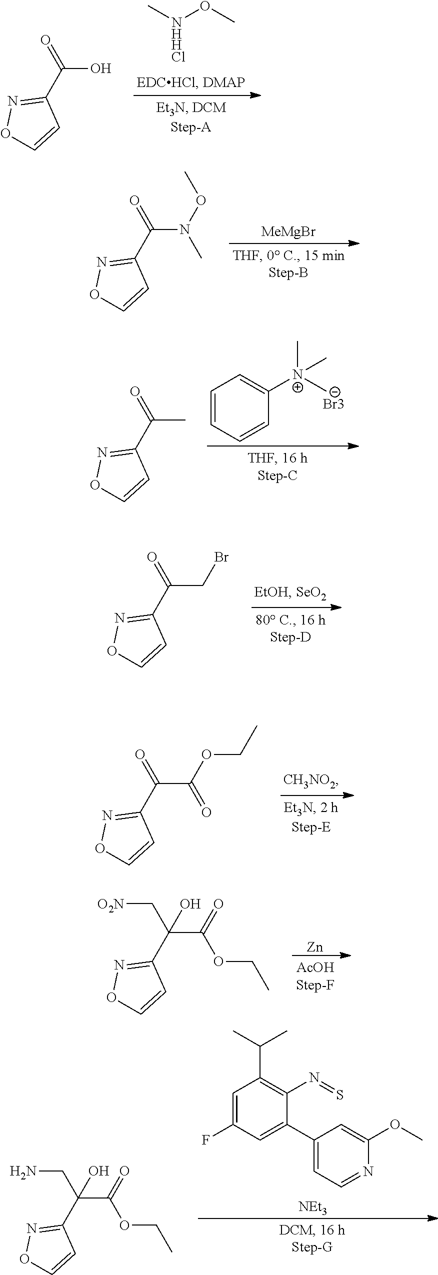

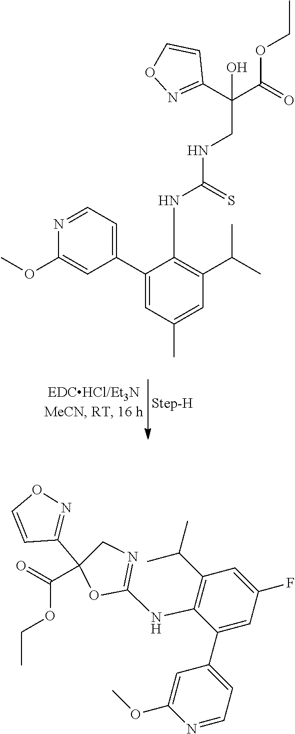 [SMILES: CCOC(=O)C(O)(CNC(=S)Nc1c(-c2ccnc(OC)c2)cc(C)cc1C(C)C)c1ccon1 ; CCOC(=O)C1(c2ccon2)CN=C(Nc2c(-c3ccnc(OC)c3)cc(F)cc2C(C)C)O1]

### Step A

To a stirred solution of isoxazole-3-carboxylic acid (25.0 g, 221.21 mmol) and N,O-dimethyl hydroxylamine hydrochloride (25.89 g, 265.46 mmol) in DCM (250 ml), cooled down to 0° C., was added DMAP (27.02 g, 221.21 mmol), EDC·HCl (50.96 g, 265.46 mmol), and Et₃N (46.2 ml, 331.6 mmol); the resulting mixture was allowed to warm up to RT and stirred under N₂ atmosphere for 6 hours. After complete consumption of the starting material, the reaction mixture was diluted with H₂O (200 ml) and extracted with DCM (2×500 ml). The organic layer was evaporated under reduced pressure. The resulting crude residue was purified by flash chromatography (SiO₂ column, 0-20% ethyl acetate in hexane) to afford N-methoxy-N-methylisoxazole-3-carboxamide as a pale yellow liquid (28 g, 82.3%). ¹H-NMR (400 MHz CDCl₃): δ=8.48-8.47 (d, 1H), 6.72 (s, 1H), 3.98 (s, 3H), 3.40 (s, 3H). MS: 156.90 [M+H]⁺.

### Step B

To a stirred solution of the compound from step-A (6.0 g, 38.42 mmol) in dry THF (60 ml), cooled down to 0° C., was added methyl magnesium bromide (3M solution in diethyl ether, 19.2 ml, 57.64 mmol) dropwise over a period of 10 minutes under N₂ atmosphere. After complete consumption of the starting material, the reaction mixture was quenched with ice water, extracted with diethyl ether (2×200 ml), dried over Na₂SO₄, filtered and evaporated under reduced pressure (with a water bath maintained at room temperature) to afford 1-(isoxazol-3-yl) ethan-1-one as a pale yellow liquid (4.5 g, crude). MS: 112.01 [M+H]⁺.

### Step-C:

To a stirred solution of the compound from step-B (4.5 g, 40.50 mmol) in THF (45 ml) was added phenyltrimethylammonium tribromide (16.74 g, 44.55 mmol) and the mixture was stirred at room temperature under N₂ atmosphere for 16 hours. After complete consumption of the starting material, as monitored by TLC, the reaction mixture was diluted with diethyl ether (30 ml), filtered through celite and washed with diethyl ether (30 ml). The filtrate was evaporated under reduced pressure to afford 2-bromo-1-(isoxazol-3-yl) ethan-1-one as a brown liquid. (9 g, crude). MS: 192 [M+2]⁺.

### Step-D:

To a stirred solution of the compound from step-C (9 g, 47.36 mmol) in ethanol (90 ml) was added SeO₂ (10.5 g, 94.73 mmol). The reaction mixture was heated to 80° C. After 16 hours stirring, the reaction mixture was filtered through celite, and the filtrate was evaporated under reduced pressure. The resulting crude residue was diluted with brine (100 ml) and extracted with ethyl acetate (200 ml). The organic layer was dried over Na₂SO₄, filtered, and evaporated under reduced pressure to afford ethyl 2-(isoxazol-3-yl)-2-oxoacetate as a brown liquid (6 g, crude). MS: 170.28 [M+H]⁺.

### Step-E:

To a stirred solution of compound from step-D (6 g, 35.47 mmol) in nitromethane (50 ml, 0.92 mol) was added NEt₃ (0.98 ml, 7.09 mmol) at room temperature and the resulting mixture was stirred at room temperature for 2 hours. After complete consumption of the starting material, as monitored by TLC, the reaction mixture was concentrated under reduced pressure. The resulting crude residue was purified by flash chromatography (Silica gel, 120 g column; 0-20% ethyl acetate hexane) to afford ethyl 2-hydroxy-2-(isoxazol-3-yl)-3-nitro propanoate as a light brown liquid (2.9 g, 35.62% over 4 steps). ¹H-NMR (400 MHz CDCl₃): δ=8.44-8.44 (d, 1H), 6.49-6.48 (d, 1H), 5.46 (d, 1H), 5.01-4.97 (d, 1H), 4.47-4.39 (m, 2H), 4.28-4.36 (m, 1H), 1.33-1.26 (t, 3H). MS: 231.10 [M+H]⁺.

### Step-F:

To a stirred solution of the compound from step-E (1.2 g, 5.21 mmol) in acetic acid (12 ml), cooled to 10° C., was added zinc dust (6.81 g, 104.26 mmol). The resulting mixture was allowed to slowly warm up to room temperature. After being stirred for 1 hour, the mixture was filtered through celite. The celite pad was rinsed with ethyl acetate (15 ml), the filtrate was evaporated under reduced pressure to afford ethyl 3-amino-2-hydroxy-2-(isoxazol-3-yl) propanoate as a yellow gummy compound (1.9 g, crude). MS: 201.27 [M+H]⁺.

### Step-G:

To a stirred solution of compound from step-F (1000 mg, 4.99 mmol) in DCM (15 ml) was added 4-(5-fluoro-3-isopropyl-2-isothiocyanatophenyl)-2-methoxypyridine Intermediate 1 (377.60 mg, 1.24 mmol) and NEt₃ (2.09 ml, 14.98 mmol) and the resulting reaction mixture was stirred at RT. After 16 hours stirring, the reaction mixture was concentrated under reduced pressure. The resulting crude residue obtained was purified by flash chromatography (SiO₂ column, 0-50% ethyl acetate in hexane) to afford ethyl 3-(3-(4-fluoro-2-isopropyl-6-(2-methoxypyridin-4-yl)phenyl)thioureido)-2-hydroxy-2-(isoxazol-3-yl) propanoate as a gummy solid. (0.530 g, 21.09% over 2 steps). MS: 503.34 [M+H]⁺.

### Step-H:

To a stirred solution of the compound from step-G (530 mg, 1.05 mmol) in acetonitrile (3 ml) was added N-(3-dimethylaminopropyl)-N′-ethylcarbodiimide hydrochloride (404.33 mg, 2.10 mmol) followed by Et₃N (0.44 ml, 3.16 mmol). After being stirred at RT for 16 hours, the reaction mixture was quenched with ice (15 ml) and extracted with DCM (2×30 ml); the organic layer was separated, dried over Na₂SO₄, filtered, and concentrated under reduced pressure. The resulting crude residue was purified by flash chromatography (SiO₂ column, 0-50% ethyl acetate in hexane to afford ethyl 2-((4-fluoro-2-isopropyl-6-(2-methoxypyridin-4-yl)phenyl)amino)-5-(isoxazol-3-yl)-4,5-dihydrooxazole-5-carboxylate as an off-white solid. (0.130 g, 26.80%). ¹H-NMR (400 MHz DMSO-d6): δ=8.88 (bs, 1H), 8.02-8.01 (d, 1H), 7.05-7.03 (d, 1H), 6.94-6.93 (d, 1H), 6.88-6.87 (d, 1H), 6.73 (s, 1H), 6.40 (s, 1H), 4.21-4.15 (m, 2H), 3.89-3.84 (bs, 5H), 3.14 (bs, 1H), 1.18-1.10 (m, 9H). MS: 469.50 [M+H]⁺.

### Example 2: enantiopure ethyl 2-{[4-fluoro-2-(2-methoxypyridin-4-yl)-6-(propan-2-yl)phenyl]amino}-5-(1,2-oxazol-3-yl)-4,5-dihydro-1,3-oxazole-5-carboxylate

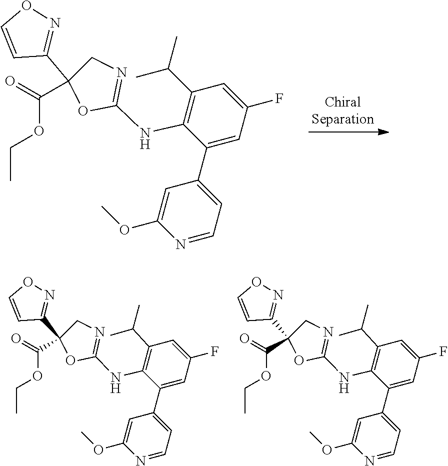

The enantiopure compound was obtained as an off white solid by chiral SFC separation starting from the racemic mixture of Example 1 (Chiral Pak IG (30*250) mm, 5μ; Co-Solvent: 10% (100% ethanol), Outlet Pressure: 100 bar, Temperature: 30° C.); First eluting peak

¹H-NMR (400 MHz DMSO-d6): δ=8.87 (bs, 1H), 8.02-8.01 (d, 1H), 7.04-7.02 (d, 1H), 6.94-6.93 (d, 1H), 6.88-6.86 (d, 1H), 6.73 (s, 1H), 6.39 (s, 1H), 4.21-4.16 (m, 2H), 3.89-3.84 (bs, 5H), 3.18-3.13 (bs, 1H), 1.18-1.11 (m, 9H). MS: 469.40 [M+H]⁺.

### Example 3: enantiopure ethyl 2-{[4-fluoro-2-(2-methoxypyridin-4-yl)-6-(propan-2-yl)phenyl]amino}-5-(1,2-oxazol-3-yl)-4,5-dihydro-1,3-oxazole-5-carboxylate

The other enantiopure compound was obtained as an off white solid, as the second eluting peak. ¹H-NMR (400 MHz DMSO-d6): δ=8.87 (bs, 1H), 8.02-8.01 (d, 1H), 7.05-7.02 (d, 1H), 6.94-6.93 (d, 1H), 6.88-6.86 (m, 1H), 6.74 (s, 1H), 6.39 (s, 1H), 4.21-4.16 (m, 2H), 3.90-3.85 (bs, 5H), 3.15 (bs, 1H), 1.18-1.11 (m, 9H). MS: 469.41 [M+H]⁺.

Following the above procedures, the following preparative examples were prepared.

| | | | 1. ¹H-NMR |
|---|---|---|---|
| Intermediate | Example | Structure | 2. MH⁺ (ESI) |
| 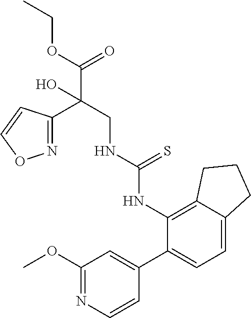 [SMILES: CCOC(=O)C(O)(CNC(=S)Nc1c(-c2ccnc(OC)c2)ccc2c1CCC2)c1ccon1] | Example 4 ethyl 2-{[5-(2- methoxy- pyridin- 4-yl)-2,3- dihydro-1H- inden-4-yl] amino}-5-(1,2- oxazol-3-yl)- 4,5-dihydro- 1,3-oxazole-5- carboxylate Racemic | 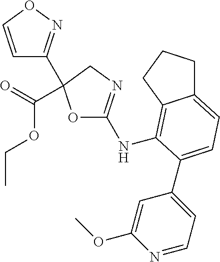 [SMILES: CCOC(=O)C1(c2ccon2)CN=C(Nc2c(-c3ccnc(OC)c3)ccc3c2CCC3)O1] | 1. ¹H-NMR (400 MHz, DMSO-d6) δ = 9.05-9.04 (d, 1H), 8.04 (brs, 1H), 7.12 (brs, 1H), 7.07-6.92 (d, 2H), 6.72-6.61 (bs, 2H), 4.26- 4.20 (q, 2H), 4.07-4.01 (bs, 2H), 3.83 (s, 3H), 2.89-2.87 (bs, 2H), 2.67 (bs, 2H), 1.97 (bs, 2H), 1.20-1.16 (t, 3H). 2. 449.50 |
| | Example 5 | | 1. ¹H-NMR (400 MHz, |
| | Enantiopure 4 | | DMSO-d6) δ = 9.05-9.04 (d, |
| | (first eluting | | 1H), 8.05-8.04 (brs, 1H), |
| | peak) | | 7.08-7.06 (brs, 1H), 6.94- |
| | | | 6.92 (d, 2H), 6.72-6.65 (bs, |
| | | | 2H), 4.26-4.20 (q, 2H), 4.08- |
| | | | 4.02 (bs, 2H), 3.83 (s, 3H), |
| | | | 2.90-2.87 (bs, 2H), 2.74- |
| | | | 2.62 (bs, 2H), 1.97 (bs, 2H), |
| | | | 1.20-1.17 (t, 3H). |
| | | | 2. 449.00 |
| | Example 6 | | 1. ¹H-NMR (400 MHz, |
| | Enantiopure 4 | | DMSO-d6) δ = 9.05-9.04 (d, |
| | (second eluting | | 1H), 8.05-8.04 (brs, 1H), |
| | peak) | | 7.08-7.06 (brs, 1H), 6.94- |
| | | | 6.93 (d, 2H), 6.72-6.65 (bs, |
| | | | 2H), 4.26-4.20 (q, 2H), 4.08- |
| | | | 4.02 (bs, 2H), 3.83-3.81 (s, |
| | | | 3H), 2.90-2.87 (bs, 2H), |
| | | | 2.67-2.62 (bs, 2H), 1.97 (bs, |
| | | | 2H), 1.20-1.16 (t, 3H). |
| | | | 2. 449.00 |
| 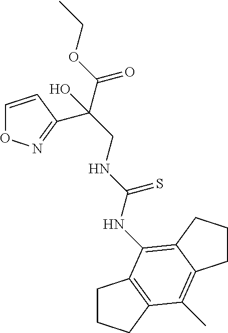 [SMILES: CCOC(=O)C(O)(CNC(=S)Nc1c2c(c(C)c3c1CCC3)CCC2)c1ccon1] | Example 7 ethyl 5- (isoxazol-3- yl)-2- ((8-methyl- 1,2,3,5,6,7- hexahydro-s- indacen-4- yl)amino)-4,5- dihydro- oxazole- 5-carboxylate Racemic | 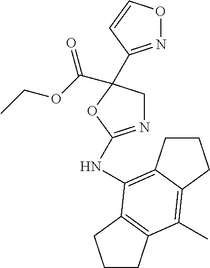 [SMILES: CCOC(=O)C1(c2ccon2)CN=C(Nc2c3c(c(C)c4c2CCC4)CCC3)O1] | 1. ¹H-NMR (400 MHz, DMSO-d6) ): δ = 9.04 (s, 1H), 8.50 (brs, 1H), 6.71 (brs, 1H), 4.25-4.23 (m, 4H), 2.75-2.72 (t, 8H), 2.07 (s, 3H), 1.97-1.93 (t, 4H), 1.22- 1.18 (t, 3H). 2. 396.30 |
| | Example 8 | | 1. ¹H-NMR (400 MHz, |
| | Enantiopure 7 | | DMSO-d6) δ = 9.04 (s, 1H), |
| | (second eluting | | 8.50 (brs, 1H), 6.71 (s, 1H), |
| | peak) | | 4.26-4.12 (m, 4H), 2.75-2.63 |
| | | | (m, 8H), 2.07-2.04 (s, 3H), |
| | | | 1.97-1.92 (m, 4H), 1.23-1.21 |
| | | | (t, 3H). |
| | | | 2. 396.26 |
| 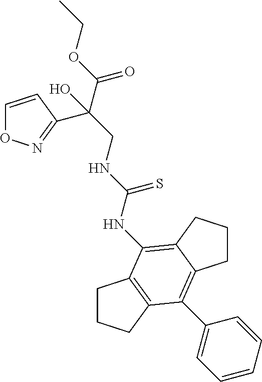 [SMILES: CCOC(=O)C(O)(CNC(=S)Nc1c2c(c(-c3ccccc3)c3c1CCC3)CCC2)c1ccon1] | Example 9 Enantiopure ethyl 5- (isoxazol-3-yl)- 2-((8-phenyl- 1,2,3,5,6,7- hexahydro-s- indacen-4- yl)amino)-4,5- dihydro- oxazole- 5-carboxylate (first eluting peak) | 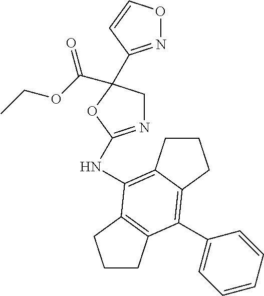 [SMILES: CCOC(=O)C1(c2ccon2)CN=C(Nc2c3c(c(-c4ccccc4)c4c2CCC4)CCC3)O1] | 1. ¹H-NMR (500 MHz, DMSO-d6) δ 9.06 (d, 1H), 8.55 (brs, 1H), 7.40 (t, 2H), 7.37-7.24 (m, 3H), 6.76 (s, 1H), 4.31-4.09 (m, 4H), 2.85-2.62 (m, 8H), 1.90 (p, 4H), 1.21 (t, 3H).. 2. 458.33 |
| | Example 16 | | 1. ¹H-NMR (500 MHz, |
| | Enantiopure | | DMSO-d6) δ 9.13-9.02 (m, |
| | ethyl 5- | | 1H), 8.55 (brs, 1H), 7.40 (t, |
| | (isoxazol-3-yl)- | | 2H), 7.30 (dd, 3H), 6.76 (s, |
| | 2-((8-phenyl- | | 1H), 4.32-4.21 (m, 3H), |
| | 1,2,3,5,6,7- | | 4.21-4.09 (m, 1H), 2.84- |
| | hexahydro-s- | | 2.61 (m, 8H), 1.90 (p, 4H), |
| | indacen-4- | | 1.21 (t, 3H). |
| | yl)amino)-4,5- | | 2. 458.33 |
| | dihydro- | | |
| | oxazole- | | |
| | 5-carboxylate | | |
| | (second eluting | | |
| | peak) | | |
| 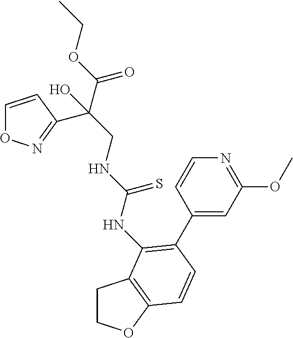 [SMILES: CCOC(=O)C(O)(CNC(=S)Nc1c(-c2ccnc(OC)c2)ccc2c1CCO2)c1ccon1] | Example 17 ethyl 5- (isoxazol-3- yl)-2-((5-(2- methoxy- pyridin- 4-yl)-2,3- dihydrobenzo- furan-4-yl) amino)-4,5- dihydro- oxazole- 5-carboxylate Racemic | 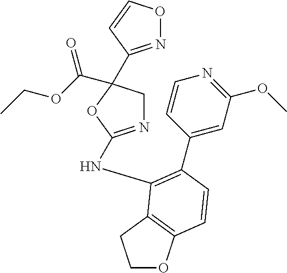 [SMILES: CCOC(=O)C1(c2ccon2)CN=C(Nc2c(-c3ccnc(OC)c3)ccc3c2CCO3)O1] | 1. ¹H-NMR at 90° C. (400 MHz, DMSO-d6) δ 8.92 (s, 1H), 8.00 (d, J = 5.2 Hz, 1H), 7.06 (d, J = 8.0 Hz, 1H), 6.93 (d, J = 4.4 Hz, 1H), 6.72 (s, 1H), 6.56 (s, 1H), 6.53-6.51 (m, 1H), 4.52 (t, J = 8.8 Hz, 2H), 4.25 (q, J = 7.2 Hz, 2H), 4.07 (d, J = 10.4 Hz, 1H), 4.03 (d, J = 10.4 Hz, 1H), 3.84 (s, 3H), 3.09-2.99 (m, 2H), 1.19 (t, J = 7.2 Hz, 3H). 2. 449.27 |
| 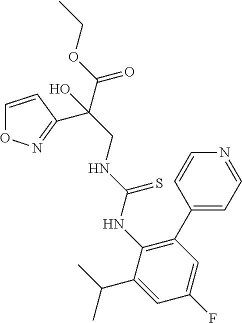 [SMILES: CCOC(=O)C(O)(CNC(=S)Nc1c(-c2ccncc2)cc(F)cc1C(C)C)c1ccon1] | Example 18 ethyl 2- ((4-fluoro- 2-isopropyl-6- (pyridin-4- yl)phenyl) amino)-5- (isoxazol-3-yl)- 4,5- dihydro- oxazole- 5-carboxylate Racemic | 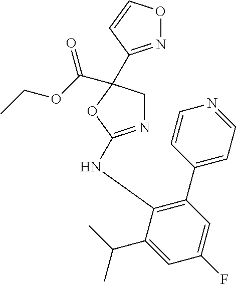 [SMILES: CCOC(=O)C1(c2ccon2)CN=C(Nc2c(-c3ccncc3)cc(F)cc2C(C)C)O1] | 1. ¹H-NMR at 90° C. (400 MHz, DMSO-d₆) δ 8.88 (s, 1H), 8.44 (d, J = 4.8 Hz, 2H), 7.34 (d, J = 5.6 Hz, 2H), 7.10- 7.03 (m, 1H), 6.92-6.87 (m, 1H), 6.40 (s, 1H), 4.21 (q, J = 6.8 Hz, 2H), 3.92-3.81 (m, 2H), 3.19-3.15 (m, 1H), 1.19- 1.11 (m, 9H) 2. 437.34 |
| 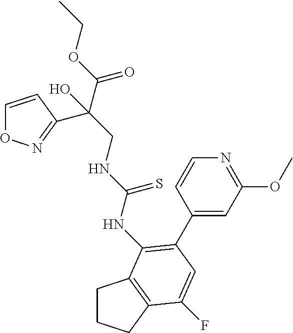 [SMILES: CCOC(=O)C(O)(CNC(=S)Nc1c(-c2ccnc(OC)c2)cc(F)c2c1CCC2)c1ccon1] | Example 19 ethyl 2-((7- fluoro-5-(2- methoxy- pyridin- 4-yl)-2,3- dihydro-1H- inden-4-yl) amino)-5- (isoxazol-3- yl)-4,5- dihydro- oxazole- 5-carboxylate Racemic | 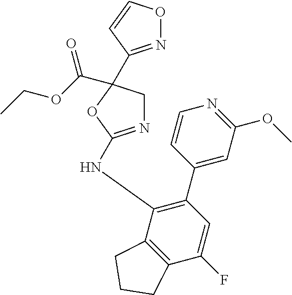 [SMILES: CCOC(=O)C1(c2ccon2)CN=C(Nc2c(-c3ccnc(OC)c3)cc(F)c3c2CCC3)O1] | 1. ¹H-NMR at 90° C. (400 MHz, DMSO-d6) δ 9.05 (s, 1H), 8.62-7.72 (m, 2H), 6.95 (d, J = 4.4 Hz, 2H), 6.75 (s, 1H), 6.65 (brs, 1H), 4.25 (q, J = 7.2 Hz 2H), 4.15-4.03 (m, 2H), 3.84 (s, 3H), 2.91 (t, J = 7.2 Hz, 2H), 2.85-2.72 (m, 2H), 2.10-1.98 (m, 2H), 1.17 (t, J = 7.2 Hz, 3H). 2. 465.40 |
| 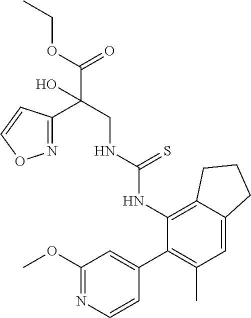 [SMILES: CCOC(=O)C(O)(CNC(=S)Nc1c2c(cc(C)c1-c1ccnc(OC)c1)CCC2)c1ccon1] | Example 20 ethyl 5- (isoxazol- 3-yl)-2-((5-(2- methoxy- pyridin-4- yl)-6-methyl- 2,3-dihydro- 1H-inden-4- yl)amino)-4,5- dihydro- oxazole- 5-carboxylate Racemic | 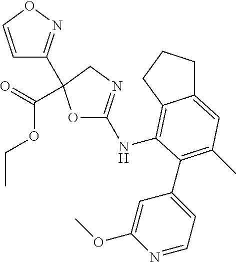 [SMILES: CCOC(=O)C1(c2ccon2)CN=C(Nc2c3c(cc(C)c2-c2ccnc(OC)c2)CCC3)O1] | 1. ¹H-NMR MHz, DMSO-d6) δ 9.05 (s, 1H), 8.06 (brs, 1H), 7.50-6.50 (m, 4H), 4.26 (q, J = 7.2 Hz, 2H), 4.10-3.93 (m, 2H), 3.84 (s, 3H), 2.85 (t, J = 6.8 Hz, 2H), 2.70-2.50 (m, 2H), 1.95 (s, 5H), 1.24-1.21 (t, J = 4.4 Hz, 3H).). 2. 463.34 |
| 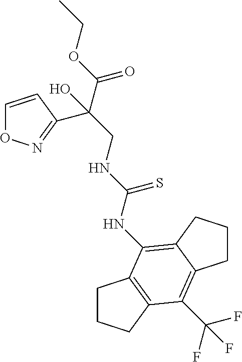 [SMILES: CCOC(=O)C(O)(CNC(=S)Nc1c2c(c(C(F)(F)F)c3c1CCC3)CCC2)c1ccon1] | Example 21 ethyl 5- (isoxazol- 3-yl)-2-((8- (trifluoro- methyl)- 1,2,3,5,6,7- hexahydro-s- indacen-4- yl)amino)-4,5- dihydro- oxazole- 5-carboxylate Racemic | 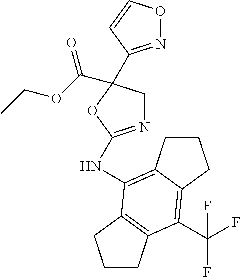 [SMILES: CCOC(=O)C1(c2ccon2)CN=C(Nc2c3c(c(C(F)(F)F)c4c2CCC4)CCC3)O1] | 1. ¹H-NMR (500 MHz, DMSO-d6) δ 9.16-8.93 (m, 1H), 7.62 (brs, 1H), 6.79 (s, 1H), 4.39-3.97 (m, 4H), 3.05-2.90 (m, 4H), 2.85- 2.53 (m, 4H), 2.05-1.88 (m, 4H), 1.20 (t, 3H). 2. 450.30 |
| | Example 22 | | 1. ¹H-NMR (400 MHz, |
| | Enantiopure 22 | | DMSO-d6 δ 9.05 (d, 1H), |
| | (first eluting | | 8.94 (s, 1H), 7.70 (s, 1H), |
| | peak) | | 6.88 (t, 1H), 6.78 (d, 1H), |
| | | | 4.51-4.34 (m, 1H), 4.31- |
| | | | 4.20 (m, 3H), 2.86 (t, 4H), |
| | | | 2.11-1.96 (m, 2H), 1.21 (t, |
| | | | 3H). |
| | | | 2. 360.18 |
| 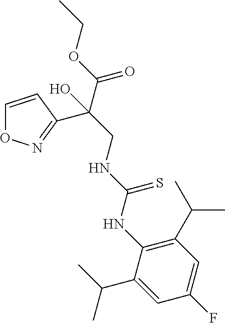 [SMILES: CCOC(=O)C(O)(CNC(=S)Nc1c(C(C)C)cc(F)cc1C(C)C)c1ccon1] | Example 23 ethyl 2- ((4-fluoro- 2,6-diiso- propylphenyl) amino)-5- (isoxazol-3- yl)-4,5- dihydro- oxazole- 5-carboxylate Racemic | 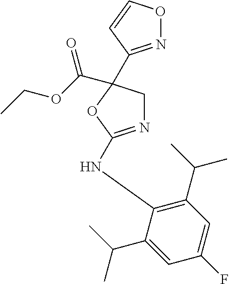 [SMILES: CCOC(=O)C1(c2ccon2)CN=C(Nc2c(C(C)C)cc(F)cc2C(C)C)O1] | 1. ¹H-NMR at 90° C. (400 MHz, DMSO-d₆) δ 8.88 (s, 1H), 6.80 (d, J = 10.0 Hz, 2H), 6.59 (s, 1H), 4.25 (q, J = 7.2 Hz, 2H), 4.13 (d, J = 11.2 Hz, 1H), 4.07 (d, J = 11.2 Hz, 1H), 3.15- 2.99 (m, 2H), 1.21 (t, J = 7.2 Hz, 3H), 1.11 (d, J = 6.8 Hz, 6H), 1.07 (d, J = 6.8 Hz, 6H) 2. 404.31 |

### Example 10 2-{[4-fluoro-2-(2-methoxypyridin-4-yl)-6-(propan-2-yl)phenyl]amino}-5-(1,2-oxazol-3-yl)-4,5-dihydro-1,3-oxazole-5-carboxylic acid

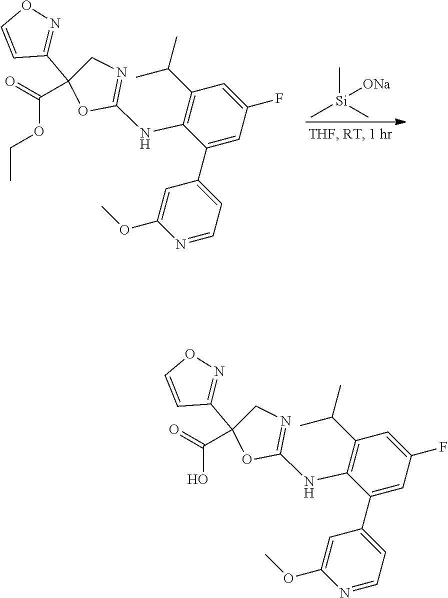

To a stirred solution of the ester of Example 1 (30 mg, 0.064 mmol) in THF (0.1 ml) was added sodium trimethylsilanolate (8.6 mg, 0.076 mmol) at room temperature and the reaction mixture was stirred at room temperature for 1 hour. After complete consumption of starting material, as monitored by TLC, the reaction mixture was concentrated under reduced pressure and purified by preparative HPLC to afford 2-((4-fluoro-2-isopropyl-6-(2-methoxypyridin-4-yl)phenyl)amino)-5-(isoxazol-3-yl)-4,5-dihydrooxazole-5-carboxylic acid as an off white solid (0.010 g, 37.31%). ¹H-NMR (400 MHz DMSO-d6): δ=8.71 (bs, 1H), 7.97-7.96 (d, 1H), 7.01-6.99 (bs, 2H), 6.85-6.83 (d, 1H), 6.73 (s, 1H), 6.31 (s, 1H), 3.81 (bs, 5H), 3.12-3.11 (bs, 1H), 1.11-1.08 (t, 6H). MS: 441.35 [M+H]⁺.

Following the saponification procedure as described in Example 10, the following preparative examples were synthesized.

| | | 1. ¹H-NMR |
|---|---|---|
| | | 2. Yield |
| Ester (starting material) | Example | 3. MH⁺ (ESI) |
| Example 3 Enantiopure (second eluting peak) | 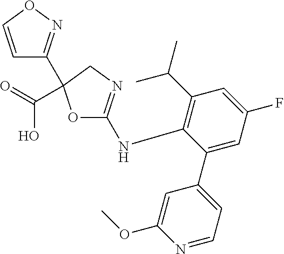 [SMILES: COc1cc(-c2cc(F)cc(C(C)C)c2NC2=NCC(C(=O)O)(c3ccon3)O2)ccn1] | 1. ¹H-NMR (80 MHz, DMSO-d6) δ = 8.54 (bs, 1H), 7.93-7.91 (d, 1H), 7.05 (bs, 1H), 6.94-6.92 (d, 1H),6.79-6.76 (m, 1H), 6.72 (s, 1H), 6.22 (s, 1H), 3.82 (bs, 3H), 3.78-3.73 (bs, 2H) 3.12 (bs, 1H), 1.09-1.06 (t, 6H). 2. 56.34% 3. 439.08. |
| | Example 11 | |
| | Enantiopure 2-{[4-fluoro-2- | |
| | (2-methoxypyridin-4-yl)-6- | |
| | (propan-2- | |
| | yl)phenyl]amino}-5-(1,2- | |
| | oxazol-3-yl)-4,5-dihydro- | |
| | 1,3-oxazole-5-carboxylic | |
| | acid | |
| 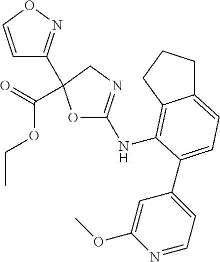 [SMILES: CCOC(=O)C1(c2ccon2)CN=C(Nc2c(-c3ccnc(OC)c3)ccc3c2CCC3)O1] | 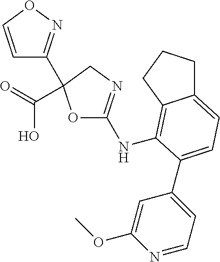 [SMILES: COc1cc(-c2ccc3c(c2NC2=NCC(C(=O)O)(c4ccon4)O2)CCC3)ccn1] | 1. ¹H-NMR (80 MHz, DMSO-d6) δ = 8.73 (s, 1H), 7.96-7.95 (d, 1H), 7.02-7.00 (d, 2H), 6.87-6.85 (bs, 2H), 6.74 (bs, 1H), 6.33-6.32(s, 1H), 3.89-3.87 (d, 1H), 3.81 (s, 3H), 3.77-3.75 (d, 1H), 2.87-2.83 (t, 2H), 2.67 (brs, 2H), 1.91 (bs, 2H). 2. 76.02% 3. 421.17 |
| Example 4 | Example 12 | |
| Racemic | 2-{[5-(2-methoxypyridin-4- | |
| | yl)-2,3-dihydro-1H-inden-4- | |
| | yl]amino}-5-(1,2-oxazol-3- | |
| | yl)-4,5-dihydro-1,3-oxazole- | |
| | 5-carboxylic acid | |
| Example 6 | Example 13 | 1. ¹H-NMR (80 MHz, DMSO-d6) |
| Enantiopure | Enantiopure 2-{[5-(2- | δ = 8.74-8.73 (d, 1H), 7.96-7.95 (d, |
| (first eluting peak) | methoxypyridin-4-yl)-2,3- | 1H), 7.02-6.86 (brs, 4H), 6.74 (bs, |
| | dihydro-1H-inden-4- | 1H), 6.33 (bs, 1H), 3.89-3.87 (d, |
| | yl]amino}-5-(1,2-oxazol-3- | 1H), 3.81 (s, 3H), 3.78-3.76 (d, |
| | yl)-4,5-dihydro-1,3-oxazole- | 1H), 2.87-2.83 (bs, 2H), 2.69-2.56 |
| | 5-carboxylic acid | (brs, 2H), 1.93-1.91 (bs, 2H). |
| | | 2. 86.16% |
| | | 3. 421.26 |
| 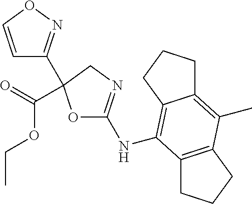 [SMILES: CCOC(=O)C1(c2ccon2)CN=C(Nc2c3c(c(C)c4c2CCC4)CCC3)O1] | 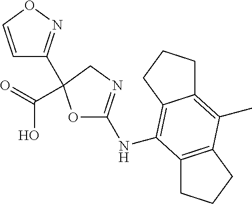 [SMILES: Cc1c2c(c(NC3=NCC(C(=O)O)(c4ccon4)O3)c3c1CCC3)CCC2] | 1. ¹H-NMR (80 MHz, DMSO-d6) δ = 8.73 (s, 1H), 6.36 (brs, 1H), 3.94-3.82 (m, 2H), 2.72-2.54 (m, 8H), 2.04 (s, 3H), 1.93-1.88 (q, 4H). 2. 40% 3. 368.27 |
| Example 7 | Example 14 | |
| | 2-[(8-methyl-1,2,3,5,6,7- | |
| | hexahydro-s-indacen-4- | |
| | yl)amino]-5-(1,2-oxazol-3- | |
| | yl)-4,5-dihydro-1,3-oxazole- | |
| | 5-carboxylic acid | |
| Example 8 | Example 15 | 1. ¹H-NMR (80 MHz, DMSO-d6) |
| Enantiopure | Enantiopure 2-[(8-methyl- | δ = 8.73 (s, 1H), 7.92 (brs, 1H), 6.36 |
| (second eluting peak) | 1,2,3,5,6,7-hexahydro-s- | (brs, 1H), 3.94-3.82 (m, 2H), 2.72- |
| | indacen-4-yl)amino]-5-(1,2- | 2.58 (m, 8H), 2.04 (s, 3H), 1.93- |
| | oxazol-3-yl)-4,5-dihydro- | 1.87 (q, 4H). |
| | 1,3-oxazole-5-carboxylic | 2. 83.3% |
| | acid | 3. 368.24 |
| 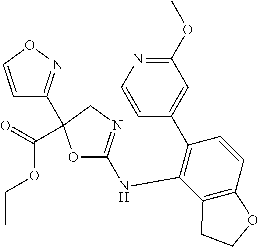 [SMILES: CCOC(=O)C1(c2ccon2)CN=C(Nc2c(-c3ccnc(OC)c3)ccc3c2CCO3)O1] | 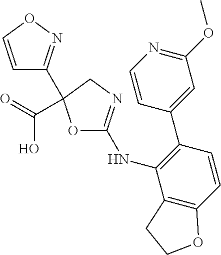 [SMILES: COc1cc(-c2ccc3c(c2NC2=NCC(C(=O)O)(c4ccon4)O2)CCO3)ccn1] | 1. ¹H-NMR at 90° C. (400 MHz, DMSO-d6) δ 8.89 (s, 1H), 7.98 (d, J = 5.2 Hz, 1H), 7.44-7.10 (brs, 2H), 7.05 (d, J = 8.0 Hz, 1H), 6.96 (d, J = 4.8 Hz, 1H), 6.72 (s, 1H), 6.54 (s, 1H), 6.53-6.47 (m, 1H), 4.50 (t, J = 8.8 Hz, 2H), 4.01 (d, J = 9.2 Hz, 1H), 3.92 (d, J = 9.2 Hz, 1H), 3.81 (s, 3H), 3.11-3.06 (m, 1H), 2.91-2.81 (m, 1H) 2. 19.6% 3. 423.31 |
| Example 17 | Example 24 | |
| | 5-(isoxazol-3-yl)-2-((5-(2- | |
| | methoxypyridin-4-yl)-2,3- | |
| | dihydrobenzofuran-4- | |
| | yl)amino)-4,5- | |
| | dihydrooxazole-5- | |
| | carboxylic acid | |
| 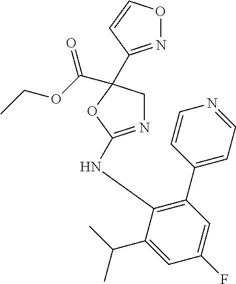 [SMILES: CCOC(=O)C1(c2ccon2)CN=C(Nc2c(-c3ccncc3)cc(F)cc2C(C)C)O1] | 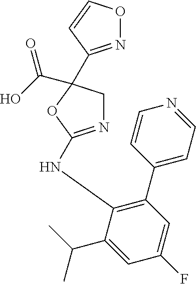 [SMILES: CC(C)c1cc(F)cc(-c2ccncc2)c1NC1=NCC(C(=O)O)(c2ccon2)O1] | 1. ¹H-NMR at 90° C. (400 MHz, DMSO-d6) δ 8.77 (s, 1H), 8.42 (d, J = 5.2 Hz, 2H), 7.36 (d, J = 5.6 Hz, 2H), 7.04 (dd, J = 10.0, 2.8 Hz, 1H), 6.88 (dd, J = 9.2, 2.8 Hz, 1H), 6.35 (s, 1H), 3.86-3.83 (m, 2H), 3.16-3.10 (m, 1H), 1.14-1.09 (m, 3H) 2. 26% 3. 411.28 |
| Example 18 | Example 25 | |
| | 2-((4-fluoro-2-isopropyl-6- | |
| | (pyridin-4- | |
| | yl)phenyl)amino)-5- | |
| | (isoxazol-3-yl)-4,5- | |
| | dihydrooxazole-5- | |
| | carboxylic acid | |
| 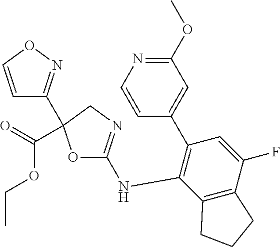 [SMILES: CCOC(=O)C1(c2ccon2)CN=C(Nc2c(-c3ccnc(OC)c3)cc(F)c3c2CCC3)O1] | 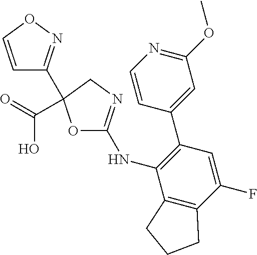 [SMILES: COc1cc(-c2cc(F)c3c(c2NC2=NCC(C(=O)O)(c4ccon4)O2)CCC3)ccn1] | 1. ¹H-NMR at 90° C. (400 MHz, DMSO-d6) δ 8.74 (d, J = 1.6 Hz, 1H), 7.98 (d, J = 4.4 Hz, 1H), 7.05- 6.90 (m, 2H), 6.87 (d, J = 9.6 Hz, 1H), 6.77 (s, 1H), 6.34 (s, 1H), 3.88 (d, J = 8.8 Hz, 1H), 3.81 (s, 3H), 3.77 (d, J = 8.8 Hz, 1H), 2.87 (t, J = 7.2 Hz, 2H), 2.74-2.72 (m, 1H), 2.65-2.58 (m, 1H), 1.99-1.96 (m, 2H). 2. 33% 3. 437.24 |
| Example 19 | Example 26 | |
| | 2-((7-fluoro-5-(2- | |
| | methoxypyridin-4-yl)-2,3- | |
| | dihydro-1H-inden-4- | |
| | yl)amino)-5-(isoxazol-3-yl)- | |
| | 4,5-dihydrooxazole-5- | |
| | carboxylic acid | |
| 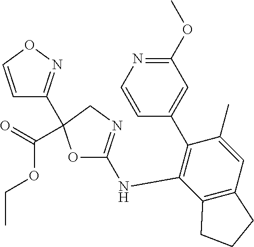 [SMILES: CCOC(=O)C1(c2ccon2)CN=C(Nc2c3c(cc(C)c2-c2ccnc(OC)c2)CCC3)O1] | 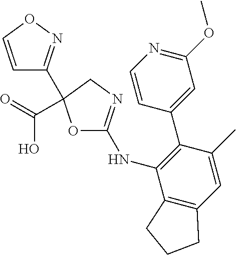 [SMILES: COc1cc(-c2c(C)cc3c(c2NC2=NCC(C(=O)O)(c4ccon4)O2)CCC3)ccn1] | 1. ¹H-NMR (400 MHz, DMSO-d6) δ 8.82 (s, 1H), 7.99 (s, 1H), 7.21- 6.80 (m, 3H), 6.51 (s, 1H), 6.40 (s, 1H), 3.88-3.82 (m, 5H), 2.82 (t, J = 7.2 Hz, 2H), 2.65-2.61 (m, 2H), 1.96 (s, 3H), 1.95-1.90 (m, 2H). 2. 44% 3. 435.32 |
| Example 20 | Example 27 | |
| | 5-(isoxazol-3-yl)-2-((5-(2- | |
| | methoxypyridin-4-yl)-6- | |
| | methyl-2,3-dihydro-1H- | |
| | inden-4-yl)amino)-4,5- | |
| | dihydrooxazole-5- | |
| | carboxylic acid | |

### Example 28: enantiopure lithium 5-(isoxazol-4-yl)-2-((8-phenyl-1,2,3,5,6,7-hexahydro-s-indacen-4-yl)amino)-4,5-dihydrooxazole-5-carboxylate

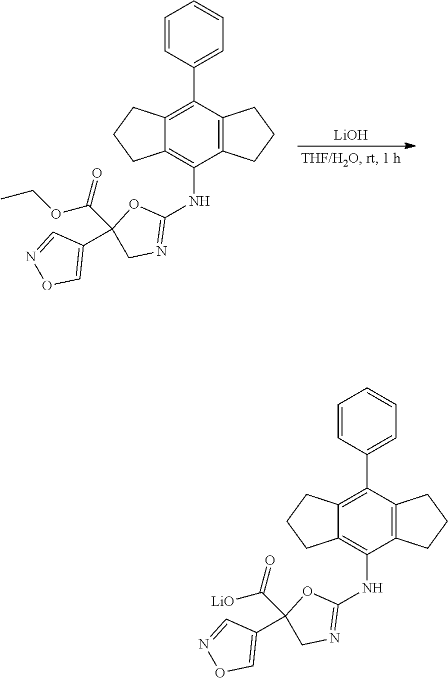

To a stirred solution of Example 16 (30 mg, 0.065 mmol) in THF (1 ml) and water (0.2 ml) was added LiOH·H₂O (3.02 mg, 0.072 mmol) and the resulting mixture was stirred at room temperature. After 1 hour stirring, the reaction mixture was concentrated under reduced pressure. The resulting residue was triturated with diethylether (5 ml) followed by n-pentane (2 ml) and dried under reduced pressure to afford enantiopure lithium 5-(isoxazol-4-yl)-2-((8-phenyl-1,2,3,5,6,7-hexahydro-s-indacen-4-yl)amino)-4,5-dihydrooxazole-5-carboxylate (27 mg, 92%) as an off-white solid. ¹H-NMR (400 MHz DMSO-d6): δ=8.74 (s, 1H), 7.39-7.24 (m, 5H), 6.39 (s, 1H), 3.93 (br s, 1H), 3.81 (br s, 1H), 2.66-2.62 (t, 8H), 1.86-1.83 (m, 4H). MS: [(M-Li)+2H]⁺.

### BIOLOGICAL ASSAY DESCRIPTION

### NLRP3 Inhibition Assay

The following assays were used to determine the inhibitory activity of test compounds on the NLRP3 inflammasome pathway using common stimuli Nigericin (Invivogen) or monosodium urate crystals (MSU) (Invivogen).

### Cell Culture

Human monocyte-like cells were cultured in RPMI-1640 Glutamax medium supplemented with 10% heat inactivated FCS and 50 U/ml penicillin-streptomycin (Life Technologies).

### NLRP3 Inflammasome Pathway Activation Assay

Human monocyte-like cells were seeded at 75000 per well in a 96-well plate and were differentiated overnight into macrophages with 10 ng/ml PMA (Phorbol Myristate Acetate). The following day, medium containing 10 ng/ml LPS (Lipopolysaccharide) were added. After 3 hours of LPS priming, concentrations of test compound in the range from 100 μM to 6 nM were added 30 min prior to NLRP3 inflammasome pathway stimulation with Nigericin 3.75 μM or MSU 200 μg/ml for 3 h.

### Measurement of IL-1β

For IL-1β quantification, supernatants were analyzed using HTRF kit according to the manufacturer's instructions (Cisbio 62HIL1BPEH). Briefly, in a 384-well ProxiPlate™ microplate, 8 μl of sample was mixed, with 4 μl of Anti-IL1β Cryptate antibody (40×) and Anti-IL1β XL antibody (40×). Then, incubated overnight at RT. Reading was done using an EnVision Reader (PerkinElmer).

IC₅₀ (concentration corresponding to 50% inhibition) were determined using GraphPad Prism 8.

The following example compounds were measured:

TABLE 1

| | | IC₅₀ |
|---|---|---|
| | IC₅₀ | Human |
| | Human | monocyte- |
| | monocyte- | like cells |
| | like cells | Nigericin |
| Examples | MSU (μM) | (μM) |
| 1 | +++ | +++ |
| 2 | +++ | +++ |
| 3 | ++ | |
| 4 | +++ | |
| 5 | ++ | |
| 6 | +++ | +++ |
| 7 | +++ | |
| 8 | +++ | +++ |
| 9 | +++ | ++ |
| 10 | +++ | |
| 11 | +++ | ++ |
| 12 | ++ | |
| 13 | +++ | + |
| 14 | ++ | |
| 15 | ++ | ++ |
| 16 | + | ++ |
| 17 | +++ | +++ |
| 18 | +++ | +++ |
| 19 | +++ | +++ |
| 20 | +++ | +++ |
| 21 | ++ | +++ |
| 22 | ++ | +++ |
| 23 | ++ | ++ |
| 24 | ++ | ++ |
| 25 | ++ | ++ |
| 26 | ++ | ++ |
| 27 | +++ | ++ |
| 28 | + | + |

Legend:
+++ IC₅₀ < 1 μM;
++ IC₅₀ 1 < x < 10 μM;
+ IC₅₀ 10 < x < 30 μM.

The tested compounds showed inhibition of IL-1beta release in human monocyte-like cells: (A) using MSU or Nigericin as activators; (B) in human whole blood assay using ATP as activator, see Table 1.

1. A compound of formula (I) 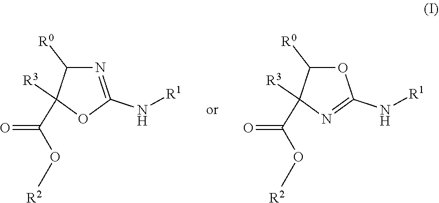
or a stereoisomer, a racemic mixture, a tautomer, a polymorph, a pharmaceutically acceptable salt, a prodrug, a hydrate, or a solvate thereof;
wherein
R⁰ is H or C₁-C₃alkyl;
R² is independently selected from the group consisting of hydrogen, C₁-C₆alkyl, and C₃-C₆cycloalkyl;
R³ is independently selected from the group consisting of 5- or 6-membered heterocycloalkyl containing one or two heteroatoms, aryl or heteroaryl containing one or two heteroatoms, wherein the heteroatoms are independently selected from N, S and O, and wherein at least one heteroatom is independently selected from O and S, and wherein the heterocycloalkyl, aryl or heteroaryl can be optionally substituted with —C₁-C₆alkyl, —O—C₁-C₆alkyl, -Hal, or —C₁-C₆alkyl-OH;
R¹ is selected from the following ring systems 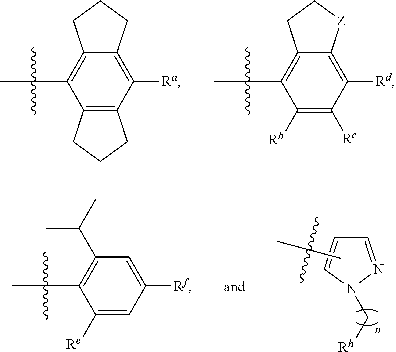
wherein 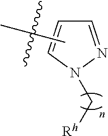
can be optionally substituted with —C₁-C₆alkyl at any available position;
Z is independently selected from the group consisting of CH₂ and O;
n is 0 or 1;
R^{a} is independently selected from the group consisting of —C₁-C₃alkyl, —C₁-C₃haloalkyl and phenyl;
R^{b} is heteroaryl optionally substituted with —C₁-C₆alkyl, —O—C₁-C₆alkyl, or —O—C₁-C₆haloalkyl;
R^{c} is independently selected from the group consisting of hydrogen or —CH₃;
R^{d} is independently selected from the group consisting of hydrogen or halogen;
R^{e} independently selected from the group consisting of —C₁-C₆alkyl, or heteroaryl, wherein heteroaryl is optionally substituted with —C₁-C₆alkyl, —C₁-C₆haloalkyl, —O—C₁-C₆alkyl, or —O—C₁-C₆haloalkyl;
R^{f} is independently selected from the group consisting of halogen and —CF₃; and
R^{h} is independently selected from the group consisting of —C₁-C₆alkyl and heteroaryl, wherein heteroaryl is optionally substituted with —C₁-C₆alkyl, —C₁-C₆haloalkyl, —O—C₁-C₆alkyl, or —O—C₁-C₆haloalkyl.

2. The compound according to claim 1, having a formula (I) 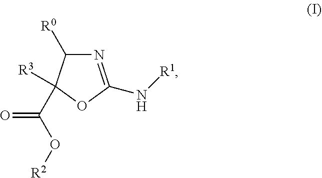
or a stereoisomer, a racemic mixture, a tautomer, a polymorph, a pharmaceutically acceptable salt, a prodrug, a hydrate, or a solvate thereof;
wherein R¹, R² are R³ are as defined in claim 1 and wherein R⁰ is H.

3. The compound according to claim 2, wherein
R¹ is selected from the following ring systems 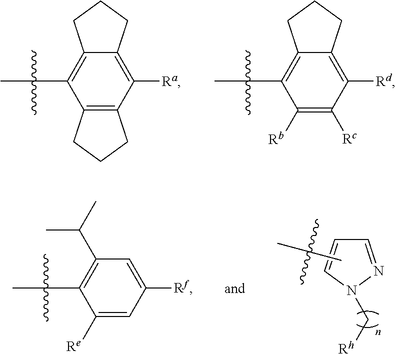
wherein 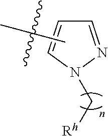
can be optionally substituted with —C₁-C₆alkyl at any available position;
R² is H or C₁-C₃alkyl;
R³ is independently selected from the group consisting of 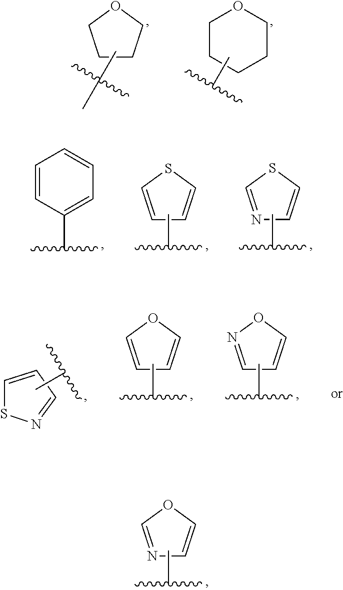
wherein each can be optionally substituted with —C₁-C₆alkyl, —O—C₁-C₆alkyl, -Hal, or —C₁-C₆alkyl-OH.

4. The compound according to claim 1, wherein
R¹ is independently selected from the following ring systems 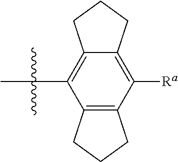
wherein R^{a} is independently selected from CH₃, CF₃ and phenyl, 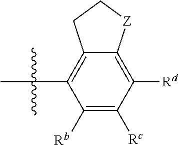
wherein R^{b} is pyridyl optionally substituted with —C₁-C₆alkyl, —O—C₁-C₆alkyl, or —O—C₁-C₆haloalkyl; Z, R^{c} and R^{d} are is as defined in claim 1, 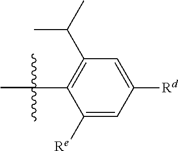
wherein R^{f} is F; R^{e} is independently selected from the group consisting of branched C₃-C₆alkyl and pyridyl optionally substituted with —C₁-C₆alkyl, —C₁-C₆haloalkyl, —O—C₁-C₆alkyl, or —O—C₁-C₆haloalkyl, 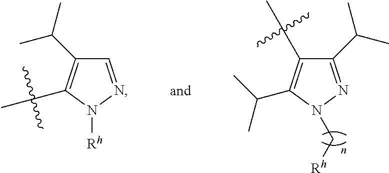
wherein R^{h} and n are as defined in claim 1.

5. The compound according to claim 1, wherein
R¹ is independently selected from the following ring systems 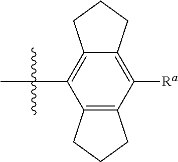
wherein R^{a} is CH₃, CF₃ or phenyl, 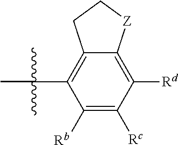
wherein R^{b} is pyridyl optionally substituted with —C₁-C₆alkyl, —O—C₁-C₆alkyl, or —O—C₁-C₆haloalkyl; Z is CH₂ or O, R^{c} and R^{d} are is as defined in claim 1, or 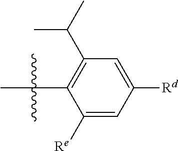
wherein R^{f} is F; R^{e} is independently selected from isopropyl and pyridyl, wherein the pyridyl can be optionally substituted with —C₁-C₆alkyl, —C₁-C₆haloalkyl, —O—C₁-C₆alkyl, or —O—C₁-C₆haloalkyl.

6. The compound according to claim 1, which is selected from 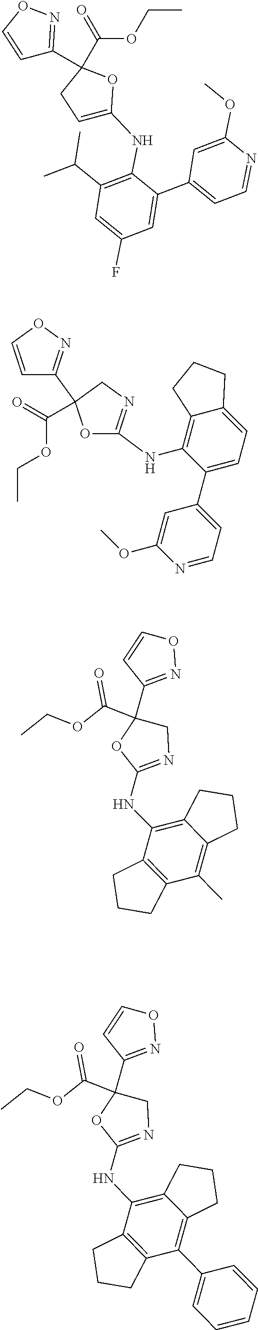 [SMILES: CCOC(=O)C1(c2ccon2)CC=C(Nc2c(-c3ccnc(OC)c3)cc(F)cc2C(C)C)O1 ; CCOC(=O)C1(c2ccon2)CN=C(Nc2c(-c3ccnc(OC)c3)ccc3c2CCC3)O1 ; CCOC(=O)C1(c2ccon2)CN=C(Nc2c3c(c(C)c4c2CCC4)CCC3)O1 ; CCOC(=O)C1(c2ccon2)CN=C(Nc2c3c(c(-c4ccccc4)c4c2CCC4)CCC3)O1] 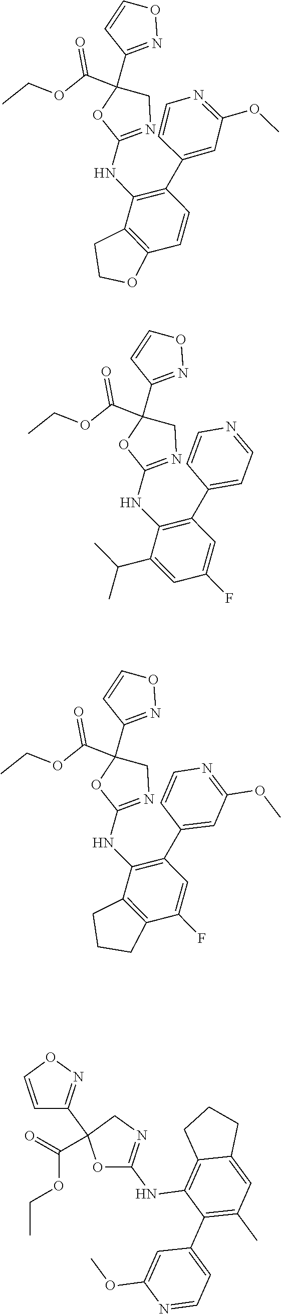 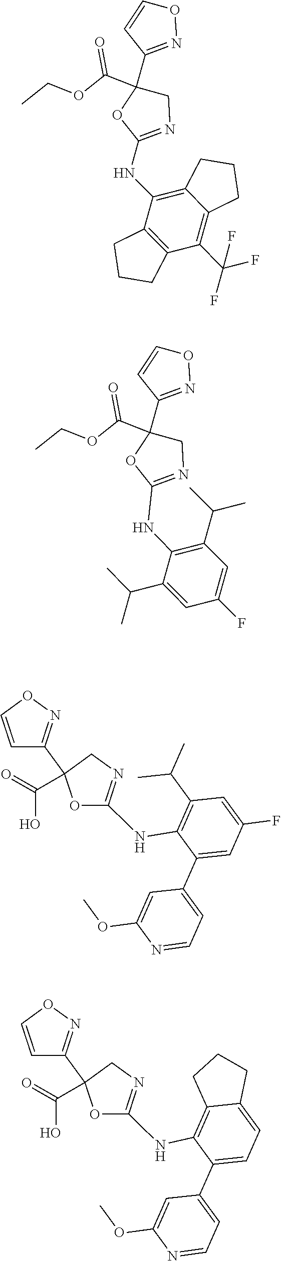 [SMILES: CCOC(=O)C1(c2ccon2)CN=C(Nc2c3c(c(C(F)(F)F)c4c2CCC4)CCC3)O1 ; CCOC(=O)C1(c2ccon2)CN=C(Nc2c(C(C)C)cc(F)cc2C(C)C)O1 ; COc1cc(-c2cc(F)cc(C(C)C)c2NC2=NCC(C(=O)O)(c3ccon3)O2)ccn1 ; COc1cc(-c2ccc3c(c2NC2=NCC(C(=O)O)(c4ccon4)O2)CCC3)ccn1] 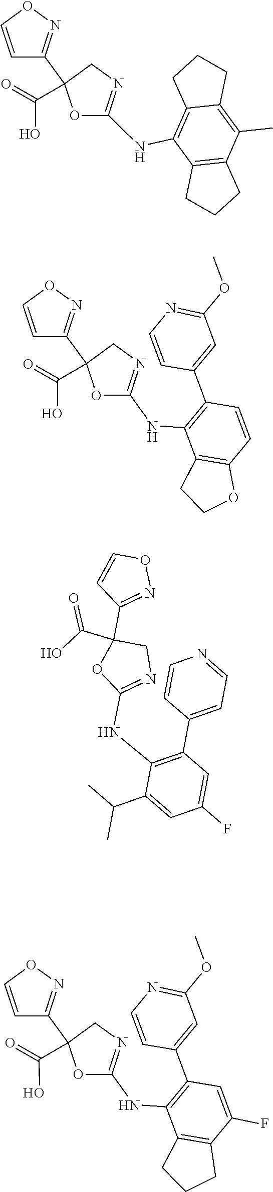 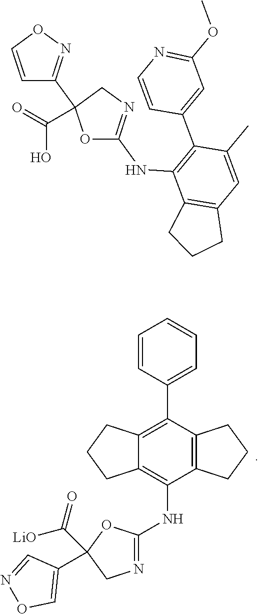
or a stereoisomer, a racemic mixture, a tautomer, a polymorph, a pharmaceutically acceptable salt, a prodrug, a hydrate, or a solvate thereof.

7. A pharmaceutical composition comprising a compound as defined in claim 1, or a stereoisomer, a racemic mixture, a tautomer, a polymorph, a pharmaceutically acceptable salt, a prodrug, a hydrate, or a solvate thereof, and comprising at least one pharmaceutically acceptable carrier, diluent, adjuvant or excipient.

8. (canceled)

9. The compound according to claim 1, or a stereoisomer, a racemic mixture, a tautomer, a polymorph, a pharmaceutically acceptable salt, a prodrug, a hydrate, or a solvate thereof, for use in the treatment, alleviation or prevention of a disease, or a disorder or an abnormality which is responsive to the modulation of a component of the NLRP3 inflammasome pathway and/or which is responsive to the modulation of IL-1 beta and/or IL-18 levels.

10. The compound for use according to claim 9, wherein the modulation is the reduction and/or inhibition of IL-1 beta.

11. The compound for use according to claim 9, wherein the component of the inflammasome pathway is NLRP3 inflammasome.

12. The compound for use according to claim 9, wherein the activation of NLRP3 inflammasome pathway is inhibited.

13. The compound for use according to any one of claim 9, wherein the disease, the disorder or the abnormality is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, Edema (DME), Geographic Atrophy (GA), rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, including Coronavirus-associated respiratory distress syndrome (CARDS), and traumatic brain injury; preferably the disorder is selected from Alzheimer's disease, Parkinson's disease, amyotrophic lateral sclerosis, demyelination, multiple sclerosis, viral encephalitis, epilepsy, stroke, traumatic brain injury, spinal cord injury, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), acute kidney disease, chronic kidney disease, lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs), acne, atopic dermatitis and hidradenitis suppurativa (HS).

14. The compound for use according to claim 13, wherein the disease, the disorder or the abnormality is selected from Alzheimer's disease, Parkinson's disease, multiple sclerosis, cryopyrin-associated periodic syndromes (CAPS), non-alcoholic fatty liver disease (NAFLD), non-alcoholic steatohepatitis (NASH), hidradenitis suppurativa (HS), chronic kidney disease and gout.

15. Use of a compound according claim 1, or a stereoisomer, a racemic mixture, a tautomer, a polymorph, a pharmaceutically acceptable salt, a prodrug, a hydrate, or a solvate thereof.

16. A compound of formula (II) 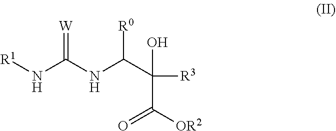
or a stereoisomer, a racemic mixture, a tautomer, a polymorph, a pharmaceutically acceptable salt, a prodrug, a hydrate, or a solvate thereof,
wherein
W is S or O;
R⁰, R¹, R² and R³ are as defined in claim 1.

17. A method of making a compound of formula (I) according to claim 1, comprising the step of cyclization of a compound of formula (II), or a stereoisomer, a racemic mixture, a tautomer, a polymorph, a pharmaceutically acceptable salt, a prodrug, a hydrate, or a solvate thereof, in the presence of a condensation agent 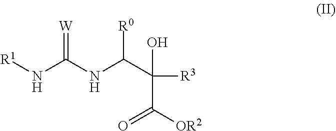
wherein
W is S or O;
R⁰, R¹, R² and R³ are as defined in claim 1.
